(19) United States
(12) Patent Application Publication
(10) Pub. No.: US 2024/0060093 A1
(43) Pub. Date: Feb. 22, 2024

(54) SYSTEMS AND METHODS FOR METHANOL PRODUCTION

(71) Applicant: WasteFuel Global LLC, Santa Monica, CA (US)

(72) Inventors: Mario E. De La Ossa, JR., New York, NY (US); Roberto Francisco Iannone, Houston, TX (US); Steven Andrew Leppard, Bellaire, TX (US); Ralph Maxwell Stock, Salem, OR (US); Charles James Thompson, League City, TX (US)

(21) Appl. No.: 18/450,986

(22) Filed: Aug. 16, 2023

Related U.S. Application Data
(60) Provisional application No. 63/398,380, filed on Aug. 16, 2022.

(51) Int. Cl. C12P 5/02; C07C 29/151; C01B 3/34; C02F 11/04; C02F 3/28; B09B 3/65

(52) U.S. Cl. CPC C12P 5/023; C07C 29/1518; C01B 3/34; C02F 11/04; C02F 3/28; B09B 3/65; C01B 2203/0233; C01B 2203/1241; C01B 2203/061; C02F 2103/36

(57) ABSTRACT

Systems and methods for producing methanol are provided. A reformer feed gas including methane and carbon dioxide is provided to a methane reformer, which produces a synthesis gas. The synthesis gas includes a first portion of hydrogen, a second portion of carbon monoxide, and a third portion of carbon dioxide. The methane is present in the reformer feed gas in an amount of between 20 volume percent (vol %) and 90 vol % and the carbon dioxide is present in the reformer feed gas in an amount of between 10 vol % and 80 vol %. The synthesis gas is subjected to a methanol synthesis reactor. The methanol synthesis reactor is utilized to produce a methanol product and a purge gas stream from the synthesis gas.

### CROSS-REFERENCE TO RELATED APPLICATION

The present application claims priority to U.S. Provisional Patent Application No. 63/398,380, entitled “Systems and Methods for Methanol Production,” filed Aug. 16, 2022, which is hereby incorporated by reference in its entirety for all purposes.

### TECHNICAL FIELD

The present disclosure relates generally to systems and methods for methanol production.

### BACKGROUND

New sources of generating, converting, or storing energy are based on an inevitable scarcity of fossil fuels, such as petroleum-based fuels. One conventional solution introduces fossil natural gas, which is primarily methane, to a methane reformer in order to produce a synthesis gas, which produces a mixture of hydrogen, carbon monoxide, and carbon dioxide, used for the production of methanol. Moreover, this natural gas includes greater than 90 volume percent methane, which makes natural gas an ideal feed gas for producing methanol through the methane reformer. See Littlefield et al., 2017, “Synthesis of Recent Ground-Level Methane Emission Measurements from the US Natural Gas Supply Chain,” Journal of Cleaner Production, 148, pg. 118-126. However, subjecting the natural gas to the methane reformer in this manner does not offset the unsatisfactory environmental aspects.

Coinciding with this need to shift away from fossil fuels is a rapid urbanization and industrialization causing an unprecedented worldwide rise in the generation of municipal solid waste (MSW). For instance, the annual generation of municipal solid waste (MSW) across major metropolitan cities worldwide is estimated to rise from 1.3 billion tons in 2012 to 2.2 billion tons in 2025. See Gundupalli et al., 2017, “A Review on Automated Sorting of Sourced-separated Municipal Solid Waste for Recycling,” Waste Management, 60, pg. 56-74.

Given this duality of needing additional energy sources with an increased generation of MSW, conventional solutions have attempted to utilize MSW to produce methanol, where such methanol, in turn, is used as a fuel or a fuel additive. However, such prior utilizations of MSW have failed to economically achieve this methanol production at useful scales. For instance, conventional, commercial-scaled methanol plants produce about 250 tons to about 300 tons of methanol per day. At this scale, the conventional methanol plants can harness sufficient energy from a methane reformer to drive the compressor drivers used in methanol production. Yet, methane, which is an ingredient utilized to produce methanol, is thermodynamically stable and requires a significant amount of energy (e.g., heat and a certain temperature) in order to decompose into reactants of hydrogen and carbon monoxide. Accordingly, in order to produce this energy within conventional, commercial scale methane plants, particularly the methane reformer, must produce sufficient excess heat, such as in the form of excess steam, to power the mechanical drivers. In this way, conventional methanol plants drive large compressors (e.g., upwards of three feet diameter) with excess heat from the process. However, such compressors are not efficient at smaller scale.

Furthermore, when utilizing MSW, such as MSW that generates gaseous material sourced from a landfill, the MSW in situ is problematic to use for methanol production. For instance, to control odors locally at the landfill, the landfill is overdrawn causing atmospheric air to be drawn into the landfill. Oxygen from the air drawn into the landfill is consumed during a biogas production process. However, nitrogen from the air drawn into the landfill will contaminate the biogas (e.g., landfill gas). As such, biogas sourced from the landfill is difficult to purify to a sufficient quality for methanol production utilization because the methane reformer is sensitive to the steam to carbon ratio, which is affected by the presence of the above-identified impurity. When the steam to carbon ratio is offset by the impurities, a catalyst of the steam reformer becomes carbonized, effectively irreversibly retarding conversion of methane in the biogas feed to hydrogen and carbon monoxide by the steam former. Furthermore, inert gases adversely buildup in the methanal synthesis reactor, which requires a large purge gas stream in order to moderate compression requirements for a recycle loop with the attendant large loss of reactants along with the purge gas stream.

Furthermore, conventional solutions have attempted to utilize a variety of waste streams that include other solid wastes, such as human waste, in order to produce biogas through anaerobic digestion. However, such conventional solutions have failed to produce a biogas with sufficient carbon content that can be utilized for efficient methanol production at scale.

The information disclosed in this Background of the Invention section is only for enhancement of understanding of the general background of the invention and should not be taken as an acknowledgement or any form of suggestion that this information forms the prior art already known to a person skilled in the art.

### SUMMARY

Given the above background, what is needed in the art are systems and methods for producing methanol.

Accordingly, various aspects of the present disclosure are directed to providing systems and methods for methanol production, such as producing a reformer feed gas and/or a methanol product.

In some embodiments, the systems and methods of the present disclosure produce the reformer feed gas and/or the methanol product from municipal solid waste (MSW), which provides significant economic and environmental advantages. For instance, the utilization of the systems and methods of the present disclosure reduces dependence on fossil fuels, provides an energy efficient system with a low emissions profile, reduces the biogas potential of MSW entering landfills, and the like.

One aspect of the present disclosure is directed to providing a method for producing methanol. The method includes providing a reformer feed gas that includes methane and carbon dioxide to a methane reformer. From this, the method produces a synthesis gas that includes a first portion of hydrogen, a second portion of carbon monoxide, and a third portion of carbon dioxide. The methane is present in the reformer feed gas in an amount of between 20 volume percent (vol %) and 90 vol %. Moreover, the carbon dioxide is present in the reformer feed gas in an amount of between 10 vol % and 80 vol %. The method further includes subjecting the synthesis gas to a methanol synthesis reactor. Furthermore, the method includes using the methanol synthesis reactor to produce a methanol product and a purge gas stream from the synthesis gas.

In some embodiments, the reformer feed gas further includes a third portion that is not reactive during the using the methanol synthesis reactor.

In some embodiments, the third portion includes a silicone compound, sulfur, a sulfur compound, or a combination thereof.

In some embodiments, the reformer feed gas is a biogas.

In some embodiments, a first portion of the reformer feed gas includes a first gas mixture obtained from a landfill gas service provider.

In some embodiments, the reformer feed gas includes a second gas mixture obtained from a municipal solid waste (MSW).

In some embodiments, the MSW includes a food waste, a garden waste, a wood waste, a crop waste, a food manufacture byproduct, a slaughterhouse waste, a used food fat, a used edible oil, a used grease from food, a manure, a biosolid, a sewage sludge, or a combination thereof.

In some embodiments, the providing the reformer feed gas further includes utilizing a first portion of the MSW as an energy source for the methane reformer.

In some embodiments, prior to the utilizing the first portion, the first portion of the MSW is subjected to a pyrolysis process.

In some embodiments the providing the reformer feed gas further includes utilizing a landfill gas stream as the energy source for the methane reformer.

In some embodiments, the reformer feed gas includes a landfill gas stream.

In some embodiments, the method further includes adjusting the reformer feed gas to have between 40 vol % and 85 vol % methane prior to the providing the reformer feed gas to the methane reformer.

In some embodiments, the method further includes adjusting the reformer feed gas to have between 15 vol % and 50 vol % carbon dioxide prior to the providing the reformer feed gas to the methane reformer.

In some embodiments, the reformer feed gas consists of less than 5 vol % fossil natural gas.

In some embodiments, the reformer feed gas consists of between 75 vol % and 85 vol % methane.

In some embodiments, the reformer feed gas during the providing the reformer feed gas to the methane reformer has a first mass flow rate between 6,000 kilograms per hour (kg/h) and 15,000 kg/h.

In some embodiments, the synthesis gas during the subjecting the synthesis gas to the methanol reformer or using the methanol synthesis reactor to product the methanol product has a second mass flow rate of between 5,000 kg/h and 20,000 kg/h.

In some embodiments, the method further includes subjecting a feedstock to an anaerobic digestion process to produce the reformer feed gas.

In some embodiments, the feedstock is MSW.

In some embodiments, the feedstock includes a food waste, a garden waste, a wood waste, a crop waste, a food manufacture byproduct, a slaughterhouse waste, a used food fat, a used edible oil, a used grease from food, a manure, a biosolid, a sewage sludge, a trim waste, or a combination thereof.

In some embodiments, the feedstock consists of between 1,000 tons per day and 15,000 tons of material per day.

In some embodiments, the synthesis gas consists of between 20 vol % and 25 vol % carbon monoxide.

In some embodiments, for the synthesis gas, the first portion of hydrogen is between 66 vol % and 78 vol %, the second portion of carbon monoxide is between 14 vol % and 33 vol %, and the third portion of carbon dioxide is between 5 vol % and 20 vol %.

In some embodiments, the first portion of hydrogen is augmented by an auxiliary hydrogen supply.

In some embodiments, the using the methanol synthesis reactor to produce the methanol product provides between 60 tons of methanol product and 250 tons of methanol product a day. In some embodiments, the using the methanol synthesis reactor to produce the methanol product provides between 70 tons of methanol product and 250 tons of methanol product a day.

In some embodiments, the methane reformer includes a steam methane reformer and/or an auto-thermal reformer. In some embodiments, the steam methane reformer and the auto-thermal formal reformer are each configured to reform both carbon dioxide and methane in the same reactor.

In some embodiments, the methanol synthesis reactor is a steam-raising reactor.

In some embodiments, a carbon efficiency of the producing the methanol product from the reformer feed gas is between 95% and 99%.

In some embodiments, the subjecting the synthesis gas to the methanol synthesis reactor is conducted at a pressure of from about 1,000 pound-force per square inch (PSI) to about 3,000 PSI.

In some embodiments, the subjecting the synthesis gas to the methanol synthesis reactor and using the methanol synthesis reactor to produce the methanol product includes passing a subset of the synthesis gas through the methanol synthesis reactor for two or more passes.

In some embodiments, the passing the subset of the synthesis gas through the methanol synthesis reactor has a conversion efficiency between about 25% to about 35% each pass.

In some embodiments, the passing the subset of the synthesis gas through the methanol synthesis reactor is configured to adjust a temperature of the methanol synthesis reactor.

In some embodiments, the passing the subset of the synthesis gas through the methanol synthesis reactor is configured to adjust an impurities volume percentage of a feed stream for the methanol synthesis reactor.

In some embodiments, each pass of the two or more passes of the passing the subset includes adjusting, by a compressor, a pressure of the fourth portion of the synthesis gas.

In some embodiments, the subset of the synthesis gas through the methanol synthesis reactor has a third mass flow rate between 20,000 kg/h and 78,000 kg/h.

In some embodiments, the methanol product includes less than 10 vol % inert impurities.

In some embodiments, the method further includes recycling the purge gas stream to a source of the reformer feed gas. In some embodiments, the method further include recycling the purge gas stream to a source configured to utilize the purge gas stream for a beneficial use.

In some embodiments, the purge gas stream includes hydrogen, carbon monoxide, carbon dioxide, one or more inert compounds, or a combination thereof.

In some embodiments, the providing the reformer feed gas further includes subjecting the reformer feed gas to an oxygen removal apparatus prior to methane reformer.

In some embodiments, the recycling the purge gas stream to the source has a fourth mass flow rate between 500 kg/h to about 2,000 kg/h.

Another aspect of the present disclosure is directed to providing a method of producing a reformer feed gas. The method includes obtaining, from one or more sources, a stream of unsorted municipal solid waste (MSW). Furthermore, the method includes processing, by one or more sorting mechanisms, the stream of unsorted MSW, which produces a stream of sorted MSW. The method further includes retaining, for an epoch, a solution including a first portion of the stream of sorted MSW in an anaerobic digestion apparatus. The first portion substantially includes digestible biological solids. From this, the method produces the reformer feed gas through anaerobic digestion of the solution in the anaerobic digestion apparatus.

In some embodiments, the stream of unsorted MSW includes a food waste, a garden waste, a wood waste, a crop waste, a food manufacture byproduct, a slaughterhouse waste, a used food fat, a used edible oil, a used grease from food, a manure, a biosolid, a sewage sludge, or a combination thereof.

In some embodiments, the stream of unsorted MSW includes from about 10 weight percent (wt %) to about 30 wt % wood waste.

In some embodiments, retaining the solution produces the reformer feed gas has at a mass flow rate between 6,000 kg/h and 15,000 kg/h.

In some embodiments, the first portion of the stream of sorted MSW includes greater than or equal to 90 wt % biological waste.

In some embodiments, the stream of unsorted MSW consists of between 1,000 tons and 15,000 tons of material per day.

In some embodiments, the one or more sorting mechanisms includes one or more mechanical sorting mechanisms, one or more electromechanical sorting mechanisms, one or more manual sorting mechanisms, or a combination thereof.

In some embodiments, the one or more mechanical or electromechanical sorting mechanisms includes a hopper sorting mechanism, a bag opening sorting mechanism, a sizing filter mechanism, a shredding sorting mechanism, a trommel sorting mechanism, a three-dimensional sorting mechanism, an optical sorting mechanism, a ballistic sorting mechanism, a magnetic sorting mechanism, an eddy current sorting mechanism, a hydrothermal digestible material extraction process, or a combination thereof.

In some embodiments, the method further includes treating, by a conversion process, the reformer feed gas, which produces a treated reformer feed gas.

In some embodiments, a carbon dioxide content of the reformer feed gas is between 35 vol % and 45 vol %, and a carbon dioxide content of the treated reformer feed gas is between 20 vol % and 23 vol %.

In some embodiments, the conversion process includes removing a first portion of the reformer feed gas that is nitrogen, a second portion of the reformer feed gas that is oxygen, a third portion of the reformer feed gas that is one or more silicon compounds, a fourth portion of the reformer feed gas that is sulfur or one or more sulfur compounds, or a combination thereof.

In some embodiments, the conversion process includes one or more dehydration processes, one or more fixed media bed processes, one or more chilling processes, one or more gas compression processes, or a combination thereof.

In some embodiments, the anaerobic digestion apparatus includes a slurry stir mechanism configured to agitate the solution continuously or intermittently for the epoch.

In some embodiments, the epoch is of from about 15 days to about 55 days.

In some embodiments, during the retaining the solution, the solution is at a temperature of from about 85 degrees Fahrenheit (° F.) to about 105° F. In some embodiments, during the retaining the solution, the solution is at a temperature of from about 115° F. to about 130° F.

In some embodiments, the solution includes from about 5 wt % solid concentration to about 25 wt % solid concentration of the first portion of the stream of sorted MSW.

In some embodiments, the solution includes a purge gas stream of a methanol synthesis reactor recycled to the solution.

In some embodiments, the solution includes wastewater from a methanol purification apparatus.

In some embodiments, the purge gas stream includes a hydrogen gas mixture.

In some embodiments, the solution has a total volume of between 40,000 cubic meters (m³) and 100,000 m³.

Yet another aspect of the present disclosure is directed to providing a system for producing methanol. The system includes a sorting mechanism configured to process a stream of unsorted municipal solid waste (MSW), which produces a stream of sorted MSW. The stream of sorted MSW includes a first portion substantially including digestible biological solids. The system further includes an anaerobic digestion apparatus. The anaerobic digestion apparatus is configured to retain a solution for an epoch. The solution includes the first portion of the stream of sorted MSW and a second portion including a first product of a methanol synthesis reactor, which produces a reformer feed gas. The reformer feed gas includes methane and carbon dioxide, in which the methane is present in the reformer feed gas in an amount of between 20 volume percent (%) and 90 vol % and the carbon dioxide is present in the reformer feed gas in an amount of between 10 vol % and 80 vol %. Moreover, the system includes a steam methane reformer configured to treat the reformer feed gas, which produces a synthesis gas. The synthesis gas includes a first portion of hydrogen, a second portion of carbon monoxide, and a third portion of carbon dioxide. Furthermore, the system includes the methanol synthesis reactor configured to produce a methanol product and the first product from the synthesis gas.

In some embodiments, the steam methane reformer utilizes as an energy source a first portion of the stream of unsorted MSW including a wood waste.

In some embodiments, the steam methane reformer utilizes as the energy source a landfill gas stream.

In some embodiments, anaerobic digestion apparatus is configured to produce a second product that includes a solid. Moreover, the steam methane reformer utilizes as the energy source the second product.

In some embodiments, the first portion of hydrogen is between 60 vol % and 78 vol %, the second portion of carbon monoxide is between 14 vol % and 33 vol %, and the third portion of carbon dioxide is between 5 vol % and 20 vol %.

The systems and methods of the present disclosure have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 illustrates various modules and/or components of methanol production system, in accordance with an embodiment of the present disclosure;

FIGS. 2A and 2B collectively provides a flow chart of methods for producing a reformer feed gas, in which dashed boxes represent optional elements in the flow chart, in accordance with an embodiment of the present disclosure;

FIGS. 3A, 3B, and 3C collectively provides a flow chart of methods for producing methanol, in which dashed boxes represent optional elements in the flow chart, in accordance with an embodiment of the present disclosure;

FIG. 4 illustrates an exemplary system topology of a methanol production system, in which dashed boxes represent optional elements, in accordance with an embodiment of the present disclosure; and

FIG. 5 illustrates another exemplary system topology of a methanol production system, in accordance with an embodiment of the present disclosure.

In the figures, reference numbers refer to the same or equivalent parts of the present invention throughout the several figures of the drawing.

### DESCRIPTION OF EMBODIMENTS

The present disclosure is directed to providing systems and methods for producing methanol, such as for producing a reformer feed gas and/or a methanol product. For instance, in some embodiments, the reformer feed gas is utilized to produce a synthesis gas, which, in turn, is utilized to produce the methanol product. In some embodiments, the systems and methods of the present disclosure make use of a sorting mechanism that is configured to process a stream of unsorted municipal solid waste (MSW) and produce a stream of sorted MSW by separating material from the steam of unsorted MSW. Non-limiting examples of the sorting mechanism include a bag opening (e.g., trash bag opening or trash bag ripping) sorting mechanism, a magnetic sorting mechanism, a shedding sorting mechanism, a rotary trommel screening sorting mechanism, a hydrothermal extraction mechanism, or a combination thereof. The stream of sorted MSW includes a first portion substantially including digestible biological solids. As a non-limiting example, in some embodiments, the digestible biological solids include organic waste (e.g., vegetable waste, food waste, source separated food waste, fresh greens, cattle excreta, corn silage, vinasse, yeast, slaughterhouse waste, sewage sludge, etc.). The system further includes an anaerobic digestion apparatus that is utilized to conduct an anaerobic digestion process task. The anaerobic digestion apparatus is configured to retain a solution for an epoch. In some embodiments, the solution has a total volume of from about 50,000 cubic meters (m³) to about 100,000 m³. The solution includes the first portion of the stream of sorted MSW. In some embodiments, the solution includes a second portion including a first product of a methanol synthesis reactor. In some embodiments, the first product of the methanol synthesis reactor includes an auxiliary hydrogen stream. For instance, in some embodiments, the auxiliary hydrogen stream is utilized to enhance production of methane from carbon dioxide during anaerobic digestion. Accordingly, the anaerobic digestion produces a reformer feed gas based on the aerobic, or biological conversion, process (e.g., digestion) of the first portion of the stream of sorted MSW. In some embodiments, the anaerobic digestion produces a second product including a solid that is subjected to a pyrolysis and/or incineration process in order to produce a biochar and/or a refuse-derived fuel (RDF). The reformer feed gas includes methane and carbon dioxide, in which the methane is present in the reformer feed gas in an amount of between 20 volume percent (vol %) and 90 vol % and the carbon dioxide is present in the reformer feed gas in an amount of between 10 vol % and 80 vol %. For instance, in some embodiments, the reformer feed gas includes methane in an amount of about 60 vol % and carbon dioxide in an amount of about 38 vol %. In some embodiments, the auxiliary hydrogen stream is utilized to enhance (e.g., increase) the methane content in the reformer feed gas, such as during the biological conversion process pricing the reformer feed gas. Accordingly, in some embodiments, by having the methane in an amount of 90 vol % or less, the reformer feed gas of the present disclosure is distinct from natural gas feed. Moreover, in some embodiments, the systems and methods of the present disclosure includes a steam methane reformer configured to treat the reformer feed gas, which produces a synthesis gas. Furthermore, the systems and methods of the present disclosure include the methanol synthesis reactor configured to produce a methanol product from the synthesis gas. In some embodiments, the methanol product is produced at a rate of about 70 tons per day to about 250 tons per day, which previously could not be accomplished in an efficient manner.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first task could be termed a second task, and, similarly, a second task could be termed a first task, without departing from the scope of the present disclosure. The first task and the second task are both tasks, but they are not the same task.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

As used herein, the term “day” refers to twenty-four hours.

As used herein, the term “epoch” means a predefined period of time.

Furthermore, in some embodiments, the term “fossil natural gas” as used herein refers to a fluid extracted from geological formations, such as in an unrefined form.

Additionally, the “subject” and “user” are used interchangeably herein unless expressly stated otherwise.

The terms “steam” and “water vapor” are used interchangeably herein unless expressly stated otherwise.

Moreover, the term “stream” as used herein means any material moving or en route, directly or indirectly, from one location to another. In some embodiments, a stream is still a stream even if it is temporarily stationary for any epoch. In some embodiments, it will be understood that if the present disclosure refers to a particular stream, this does not necessarily refer to a single pipe or other physical conveyance. There are many equivalent ways to convey material from one location to another, which may include the use of multiple streams, conveyor belts, trucks, railcars, front-end loaders, or any other means of conveyance known in the art.

Furthermore, when a reference number is given an “i^{th}” denotation, the reference number refers to a generic component, set, or embodiment. For instance, an instrument termed “instrument device i” refers to the i^{th} instrument in a plurality of instruments (e.g., an instrument 110-i in a plurality of instruments 110).

As used herein, the terms “percent volume” and “volume percent,” are used interchangeably.

As used herein, the term “percent volume” and “volume percent,” mean a first volume of a constituent divided by a second volume of all constituents of a fluid.

As used herein, the terms “percent weight” and “weight percent,” are used interchangeably.

As used herein, the term “percent weight” and “weight percent,” mean a first weight of a constituent divided by a second weight of all constituents of a material.

Additionally, as used herein, the term “digest” means to break apart, such as into smaller pieces or components.

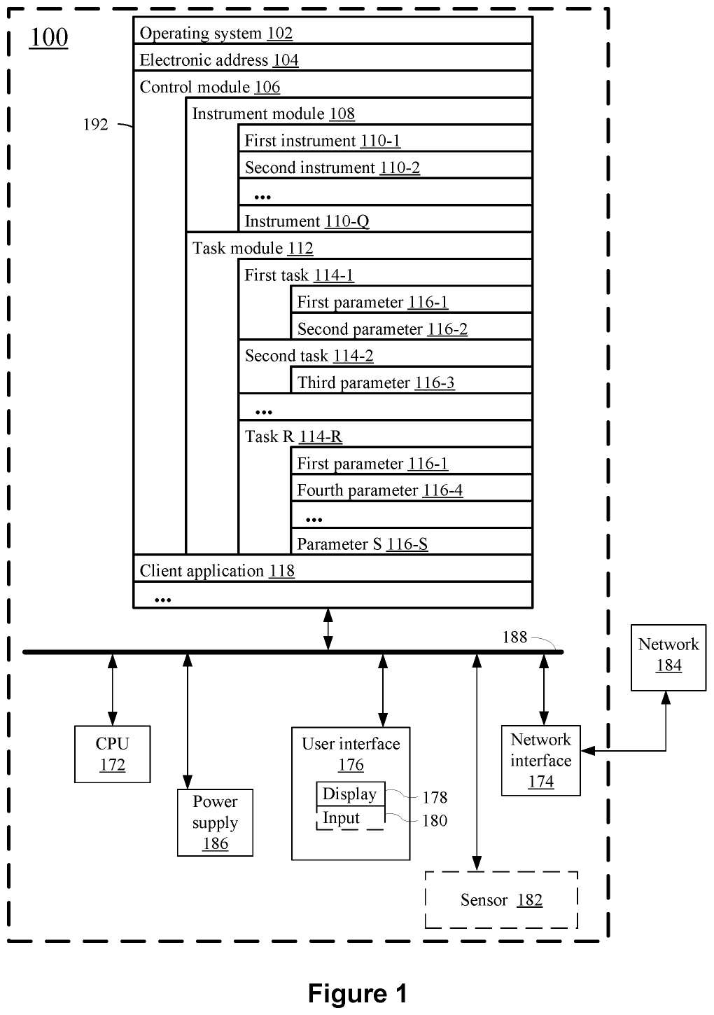

In the present disclosure, unless expressly stated otherwise, descriptions of devices and systems will include implementations of one or more computers. For instance, and for purposes of illustration in FIG. 1, a computer system 100 is represented as single device that includes all the functionality of the computer system 100. However, the present disclosure is not limited thereto. For instance, the functionality of the computer system 100 may be spread across any number of networked computers and/or reside on each of several networked computers and/or by hosted on one or more virtual machines and/or containers at a remote location accessible across a communication network (e.g., communication network 184). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 184, the illustrated devices and systems may wirelessly transmit information between each other. As such, the exemplary topology shown in FIG. 1 merely serves to describe the features of an embodiment of the present disclosure in a manner that will be readily understood to one of skill in the art.

FIG. 1 depicts a computer system for producing methanol in accordance with some embodiments of the present disclosure.

In some embodiments, the communication network 186 optionally includes the Internet, one or more local area networks (LANs), one or more wide area networks (WANs), other types of networks, or a combination of such networks.

Examples of communication networks 184 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 172, a network or other communications interface 174, and memory 192.

In some embodiments, the computer system 100 includes a user interface 176. The user interface 176 typically includes a display 178 for presenting media, such as a status of a respective instrument (e.g., first instrument 110-1, second instrument 110-2, . . . , instrument Q 112-Q of FIG. 1). In some embodiments, the display 178 is integrated within the computer systems (e.g., housed in the same chassis as the CPU 172 and memory 192). In some embodiments, the computer system 100 includes one or more input device(s) 180, which allow a subject to interact with the computer system 100. In some embodiments, input devices 180 include a keyboard, a mouse, and/or other input mechanisms. Alternatively, or in addition, in some embodiments, the display 178 includes a touch-sensitive surface (e.g., where display 178 is a touch-sensitive display or computer system 100 includes a touch pad).

In some embodiments, the computer system 100 presents media to a user through the display 178. Examples of media presented by the display 178 include one or more images, a video, audio (e.g., waveforms of an audio sample), or a combination thereof. In typical embodiments, the one or more images, the video, the audio, or the combination thereof is presented by the display 178 through a client application stored in the memory 192. In some embodiments, the audio is presented through an external device (e.g., speakers, headphones, input/output (I/O) subsystem, etc.) that receives audio information from the computer system 100 and presents audio data based on this audio information. In some embodiments, the user interface 176 also includes an audio output device, such as speakers or an audio output for connecting with speakers, earphones, or headphones.

The memory 192 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. The memory 192 may optionally include one or more storage devices remotely located from the CPU(s) 172. The memory 192, or alternatively the non-volatile memory device(s) within memory 192, includes a non-transitory computer readable storage medium. Access to memory 192 by other components of the computer system 100, such as the CPU(s) 172, is, optionally, controlled by a controller. In some embodiments, the memory 192 can include mass storage that is remotely located with respect to the CPU(s) 172. In other words, some data stored in the memory 192 may in fact be hosted on devices that are external to the computer system 100, but that can be electronically accessed by the computer system 100 over an Internet, intranet, or other form of network 184 or electronic cable using communication interface 174.

In some embodiments, the memory 192 of the computer system 100 for producing methanol stores:
an operating system 102 (e.g., ANDROID, iOS, DARWIN, RTXC, LINUX, UNIX, OS X, WINDOWS, or an embedded operating system such as VxWorks) that includes procedures for handling various basic system services;
optionally, an electronic address 104 associated with the computer system 100 that identifies the computer system 100 (e.g., within the communication network 186);
a control module 106 that controlling one or more operations conducted when producing methanol including an instrument module 108 storing a record of a plurality of instruments 110 (e.g., first instrument 110-1, second instrument 110-2, . . . , instrument 110-Q of FIG. 1) utilized for producing a methanol product and/or a reformer feed gas, and further including a task module 112 that stores a plurality of tasks 114, each task 114 defines an operation for producing methanol in accordance with one or more parameters 116 associated with a respective task 114; and
optionally, a client application 118 for presenting information (e.g., media) using a display 178 of the computer system 100.

As indicated above, an optional electronic address 104 is associated with the computer system 100. The optional electronic address 204 is utilized to at least uniquely identify the computer system 100 from other devices and components of the distributed system 100, such as other devices having access to the communications network 186. For instance, in some embodiments, the electronic address 104 is utilized to receive a request from a remote device to initiate producing methanol using the computer system 100. However, the present disclosure is not limited thereto.

An instrument 110 is an apparatus, device, mechanism, or combination thereof that conducts a specific function or functions in a system for producing methanol, such as for producing a methanol product and/or a reformer feed gas. For instance, in some embodiments, each respective instrument 110 in the plurality of instruments 110 that conducts a specific task 114 or tasks 114 in a system for producing a methanol product and/or a reformer feed gas. Examples of instruments 110 include, but are not limited to, an anaerobic digestion apparatus, a boiler (e.g., a heat recovery boiler), a burner, a compressor, a condenser, a conduit, a cooler (e.g., air cooler, gas cooler, etc.), a deaerator, a dehumidifier, a distillation unit (e.g., an atmospheric distillation unit, a vacuum distillation unit, etc.), a drum, a fan (e.g., air fan, extraction fan), a furnace, a heat exchanger (e.g., a heater, preheater, superheater, thermosiphon heater), an interchanger (e.g., reactor interchanger, etc.), an ionizer, a membrane, a pipe, a phase separator (e.g., gas-liquid phase separator), a pump, a reactor (e.g., hydrotreating reactor, desulfurization reactor, methanol reactor, etc.), a recuperator, a reformer (e.g., methane reformer), a reservoir, a saturator, a sorting mechanism (e.g., hopper sorting mechanism, bag opening sorting mechanism, sizing filter mechanism, shredding sorting mechanism, trommel sorting mechanism, three-dimensional sorting mechanism, optical sorting mechanism, ballistic sorting mechanism, magnetic sorting mechanism, eddy current sorting mechanism, a hydrothermal digestible material extraction mechanism, etc.), a valve, a vessel (e.g., activated carbon vessel, condensate vessel, etc.), or the like.

For instance, in some embodiments, the one or more instruments 110 includes a first sorting mechanism instrument 110-1 configured to process a stream of unsorted MSW, such as in order to produce a stream of sorted MSW. In some embodiments, the one or more instruments 110 include an anaerobic digestion apparatus instrument 110-2 that is configured to retain a solution. In some embodiments, the one or more instruments 110 includes a steam methane reformer instrument 110-3 configured to treat a reformer feed gas in order to produce a synthesis gas and/or a methanol product. However, the present disclosure is not limited thereto.

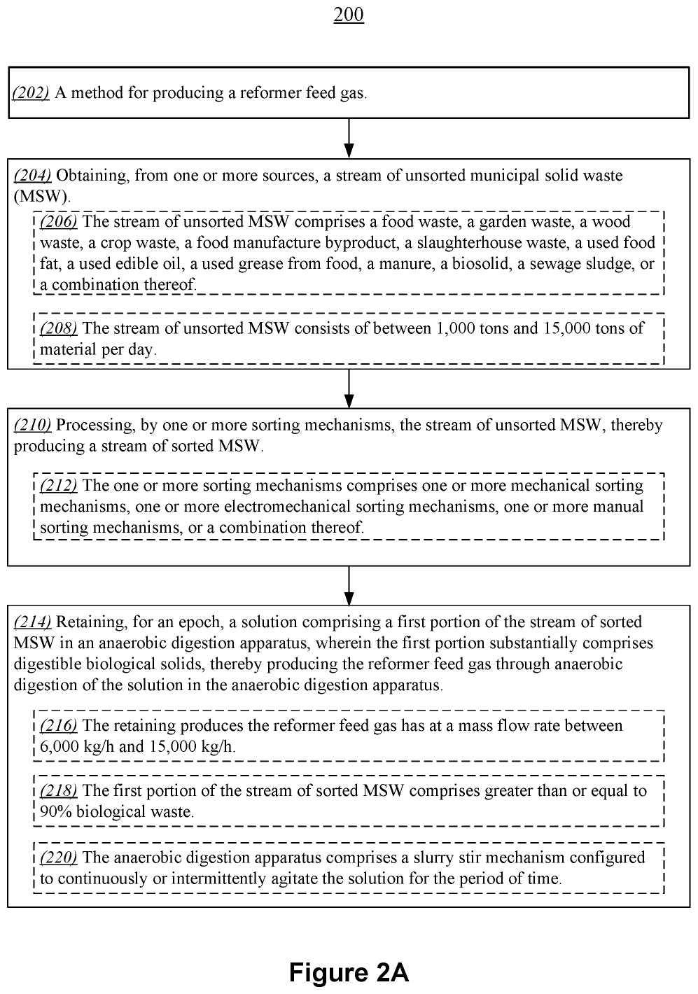

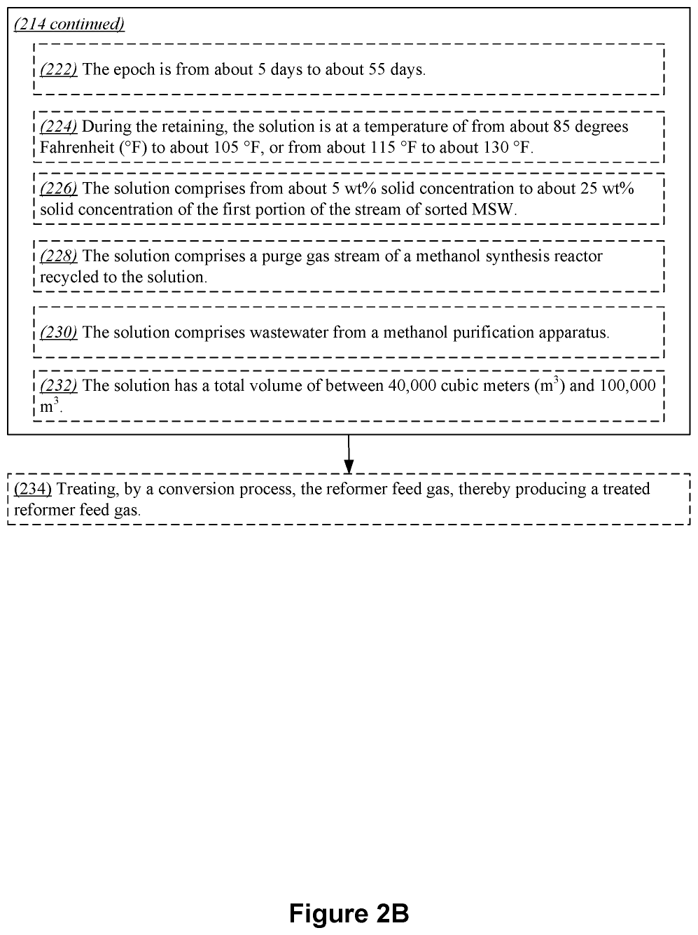

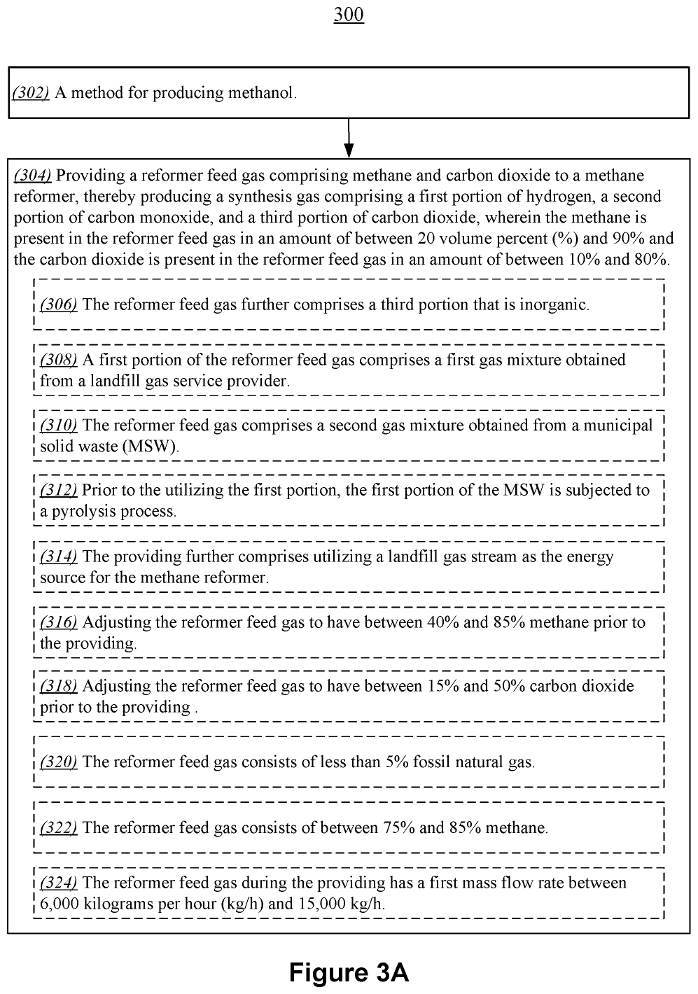

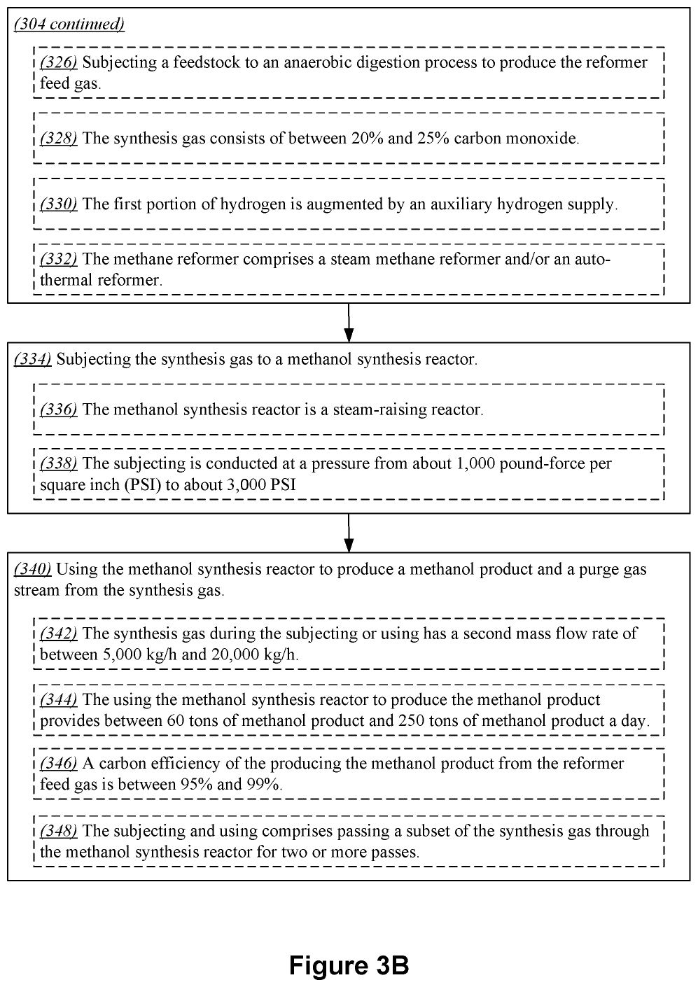

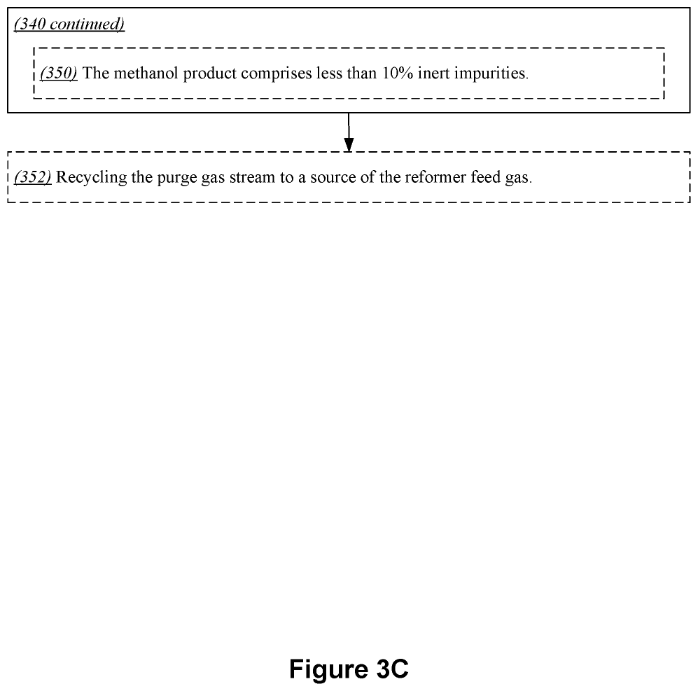

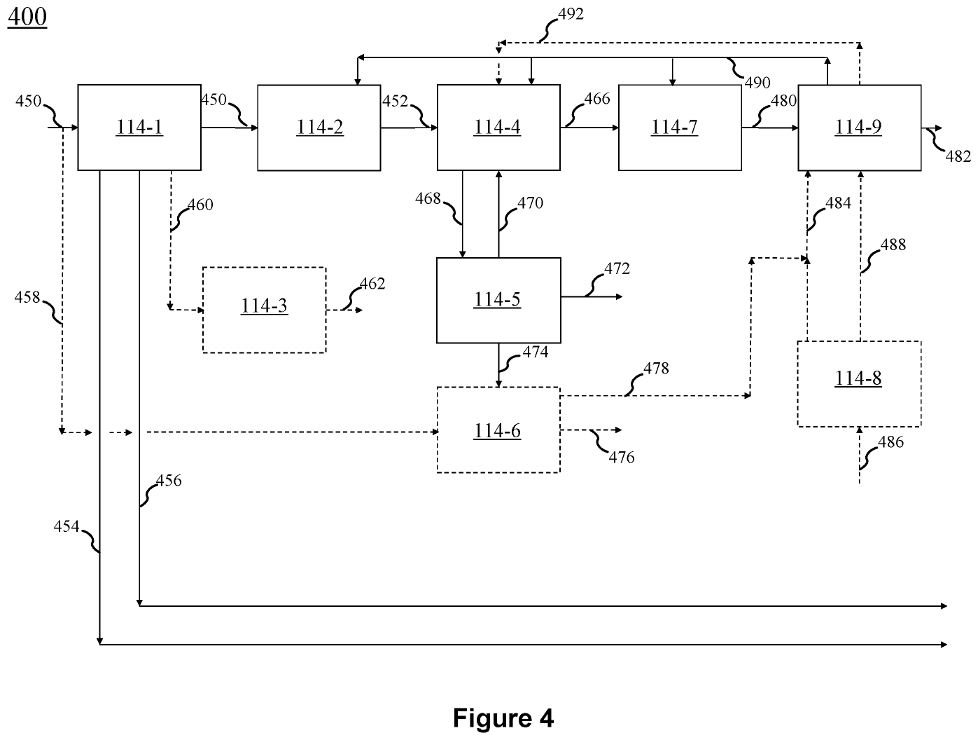

Each task 114 is associated with a function, step, or process in the production of a methanol product and/or a reformer feed gas, which is performed by a set of instruments 110. As a non-limiting example, referring briefly to FIG. 4, in some embodiments, one or more tasks 114 for producing the methanol product and/or the reformer feed gas includes a first task 114-1 associated with a first set of one or more sorting mechanism instruments 110 (e.g., block 210 of FIG. 2A, block 212 of FIG. 2A, etc.), a second task 114-2 associated with a second set of one or more sorting mechanism instruments 110 (e.g., block 210 of FIG. 2A, block 212 of FIG. 2A, etc.), a third task 114-3 associated with processing a portion of a sorted stream of MSW, a fourth task 114-4 associated with retaining a solution in an anaerobic digestion apparatus instrument 110 (e.g., block 214 of FIG. 2A, block 216 of FIG. 2A, block 218 of FIG. 2A, block 220 of FIG. 2A, block 222 of FIG. 2B, block 224 of FIG. 2B, block 226 of FIG. 2B, block 228 of FIG. 2B, block 230 of FIG. 2B, block 232 of FIG. 2B, etc.), a fifth task 114-5 associated with processing wastewater from a methanol purification apparatus, a sixth task 114-6 associated with processing a digestate or byproduct of a respective task, a seventh task 114-7 associated with a conversion process for treating a reformer feed gas, (e.g., block 234 of FIG. 2B), an eighth task 114-8 associated with a treating a landfill gas stream (e.g., block 308 of FIG. 4A), a ninth task 114-9 associated with a methanol synthesis reactor (e.g., block 334 of FIG. 3B, block 340 of FIG. 3B, etc.), or the like. For instance, in some embodiments, a task 114 is an instrument 110 configuration and/or process condition. Such instrument 110 configurations and/or process conditions include, but are not limited to, adjusting rotations per minute (RPM) of an instrument 110, changing a status of the instrument 110 from or to ON and OFF.

Moreover, each task 114 includes a set of parameters 116 used in the performance of a function by a respective instrument 110. In some embodiments, each task 114 is a logical dependency of operations that defines the function performed by the respective instrument 110. For instance, in some embodiments, the task 114 is a first operation to run a first instrument 110-1 with a first set of parameters 116 and a second task 114-2 is a second operation to run a second instrument 110-2.

As a non-limiting example, the computer system 100 configures one or more parameters 116 including configuring a flow rate parameter 116 associated with a respective instrument 110 (e.g., mass flow rate), an air to fuel ratio parameter 116, an exhaust gas temperature parameter 116, a coolant temperature parameter 116, a directional parameter 116, an oxygen percentage parameter 116, or the like.

Each of the above identified modules and applications correspond to a set of executable instructions for performing one or more functions described above and the methods described in the present disclosure (e.g., the computer-implemented methods and other information processing methods described herein, method 200 of FIGS. 2A and 2B, method 300 of FIGS. 3A, 3B, 3C, etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 192 optionally stores a subset of the modules and data structures identified above. Furthermore, in some embodiments, the memory 192 stores additional modules and data structures not described above.

It should be appreciated that the computer system 100 of FIG. 1 is only one example of a computer system 100, and that the computer system 100 optionally has more or fewer components than shown, optionally combines two or more components, or optionally has a different configuration or arrangement of the components. The various components shown in FIG. 4 are implemented in hardware, software, firmware, or a combination thereof, including one or more signal processing and/or application specific integrated circuits.

Now that a general topology of the distributed computer system 100 has been described in accordance with various embodiments of the present disclosures, details regarding some processes in accordance with FIGS. 2A and 2B will be described. Specifically, FIGS. 2A and 2B collectively illustrate a flow chart of methods (e.g., method 200) for producing a reformer feed gas (e.g., reformer feed gas 466 of FIG. 2), in accordance with embodiments of the present disclosure.

Various modules in the memory 192 of the computer system 100 perform certain processes of the methods 200 described in FIGS. 2A and 2B, unless expressly stated otherwise. Furthermore, it will be appreciated that the processes in FIGS. 2A and 2B can be encoded in a single module or any combination of modules.

Block 202. Referring to block 202 of FIG. 2A, a method 200 for producing a reformer feed gas (e.g., reformer feed gas 466 of FIG. 2) is provided.

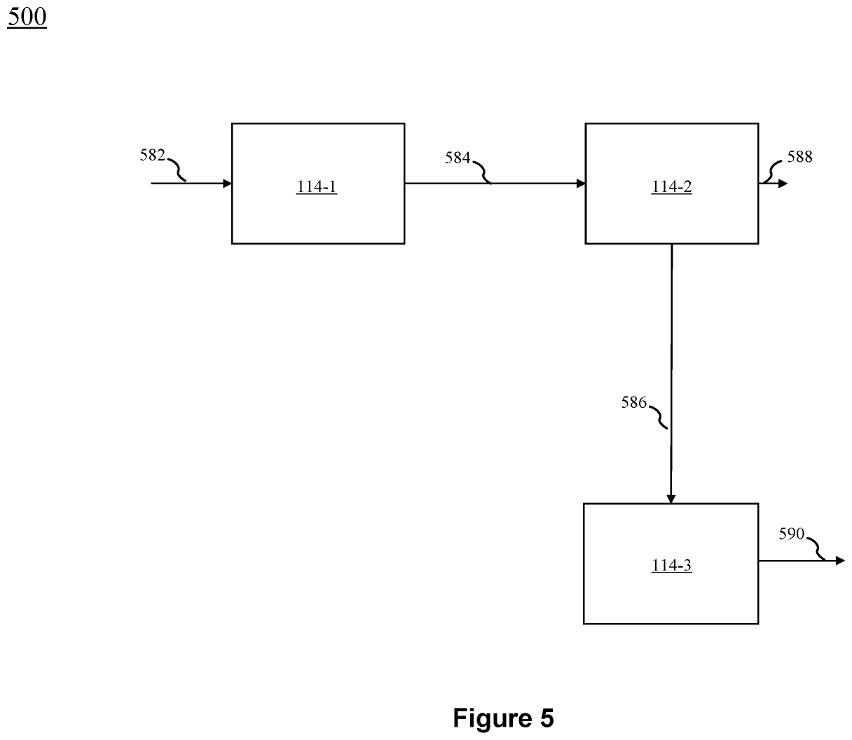

In some embodiments, the method 200 is conducted at, or in conjunction with, a computer system (e.g., distributed system 100 of FIG. 1, system 400 of FIG. 4, system 500 of FIG. 5, etc.). The computer system 100 includes one or more processors (e.g., CPU 172 of FIG. 1), and a memory (e.g., memory 192 of FIG. 1) that is coupled to the one or more processors 172. The memory 192 includes one or more programs (e.g., control module 106 of FIG. 1, client application 118 of FIG. 1, etc.) that is configured to be executed by the one or more processors 172. In other words, in some embodiments, the method 200 cannot be mentally performed because the computational complexity addressed by the method 200 requires use of the computer system.

Block 204. Referring to block 204, the method 200 includes obtaining, from one or more sources, a stream of unsorted municipal solid waste (MSW) (e.g., stream of unsorted MSW 450 of FIG. 4). In some embodiments, the one or more sources includes a waste management entity that provides waste management and/or recycling services for a population of subjects, such as a population of residents, a company, a city, or the like. For instance, in some embodiments, a respective source in the one or more sources is associated with providing a particular type of MSW depending on an origin of the MSW. As a non-limiting example, a first source is associated with household garbage and trash and a second source is associated with wastewater treatment sludge. In some embodiments, the respective source in the one or more sources is associated with industrial MSW (e.g., waste generated in governmental entities and/or private entities, educational entities, museums, libraries, archaeological zones and recreation centers, such as movie theaters and stadiums, commercial MSW, pollutant MSW, institutional MSW, constructional MSW, agricultural and animal husbandry MSW, domestic MSW, or a combination thereof). However, the present disclosure is not limited thereto. In some embodiments, a respective source in the one or more sources is associated with providing a particular type of MSW depending on a physical and/or chemical characteristic of the MSW. Additional details and information regarding the one or more sources of MSW is found at Buenrostro et al., 2001, “Classification of Sources of Municipal Solid Wastes in Developing Countries,” Resources, Conservation and Recycling, 32(1), pg. 29-41, which is hereby incorporated by reference in its entirety for all purposes.

In some embodiments, the stream of unsorted MSW 450 includes one or more material phases, such as one or more liquids, one or more solids, one or more contained gases, one or more sludges, or a combination thereof. For instance, in some embodiments, the stream of unsorted MSW 450 is a slurry that includes dissolved solids (e.g., sewage sludge solids, poultry air dissolved floatation solids, etc.), used cooking oil, or the like. In some embodiments, the MSW in situ is not made entirely solid by including entrained or absorbed liquids, or liquids in containers or other enclosed spaces. However, the present disclosure is not limited thereto.

Block 206. Referring to block 206, in some embodiments, the stream of unsorted MSW includes various types of waste. For instance, food waste, garden waste, wood waste, crop waste, food manufacture byproducts, slaughterhouse waste, used food fat, oils, greases, manure, biosolids, sewage sludge, or a combination thereof.

By way of example, in some embodiments, the food waste includes material intended for human consumption that is subsequently discharged, degraded, contaminated, or a combination thereof. As a non-limiting example, in some embodiments, the food waste includes one or more meats (e.g., pork, poultry, etc.), one or more vegetables, one or more fruits, one or more breads, one or more bones (e.g., fish bones, animal bones, etc.), eggshell, or the like. For instance, in some embodiments, the food waste includes any food or inedible materials removed from (e.g., lost to and/or diverted from) a food supply chain to be recovered or disposed (e.g., including composted, crops ploughed in/not harvested, anaerobic digestion, bio-energy production, co-generation, incineration, disposal to sewer, landfill, or discarded to sea). Accordingly, in some embodiments, the food waste includes low quality fruits and/or vegetables (e.g., culls), damaged productions left in the field, good products or co-products with a low or absent commercial value, uncooked food (e.g., expired food), or a combination thereof, such as process losses, product damaged during transport, spoilage, plate scrapings and surplus food cooked and not used, food discarded in packaging, for, and the like. Additional details and information regarding suitable food waste is found at Griotto et al., 2015, “Food Waste Generation and Industrial Uses: A Review,” Waste Management, 45, pg. 32-41; Papargyropoulou et al., 2014, “The Food Waste Hierarchy as a Framework for the Management of Food Surplus and Food Waste,” Journal of Cleaner Production, 76, pg. 106-115, each of which is hereby incorporated by reference in its entirety for all purposes.

In some embodiments the unsorted MSW includes easte generated by households, commercial establishments, and institutions within a municipality. In some embodiments, the unsorted MSW comprises or consists of organic waste. Organic waste includes food waste, yard waste, and other biodegradable materials. In some embodiments, the unsorted MSW does not include paper or cardboard. In some embodiments, the unsorted MSW does not include plastics, glass, metals, textiles or E-waste.

In some embodiments, garden waste, sometimes referred to as yard waste, of the stream of unsorted MSW includes waste that is generated from a garden (e.g., a personal garden, a commercial garden, etc.) and/or a park (e.g., a public playground, a national forest and park, etc.), such as from landscaping practices and/or environmental damage. As a non-limiting example, in some embodiments, the garden waste includes organic and/or inorganic materials. For instance, in some embodiments, the organic material of the garden waste includes clippings (e.g., grass clippings), cuttings (e.g., hedge cuttings), pruning, leaves, wood, flowers, or a combination thereof. In some embodiments, the inorganic material of the garden waste includes soil and/or stone (e.g., rock). Accordingly, in some such embodiments, the garden waste includes a first portion of about 25 wt % to about 55 wt % cellulose, a second portion of about 20 wt % to about 30 wt % hemicelluloses, and a third portion of about 25 wt % to about 30 wt % lignin. Moreover, in some embodiments, the energy value (e.g., gross caloric value) of the garden waste is about 13 megajoules per kilogram (MJ kg) to about 22 MJ kg⁻¹, about 14 MJ kg⁻¹ to about 21 MJ kg, about 15 MJ kg to about 20 MJ kg⁻¹, about 16 MJ kg⁻¹ to about 19 MJ kg⁻¹, or about 17 MJ kg to about 18 MJ kg. In some embodiments, the energy value of the garden waste is about 13 MJ kg, about 14 MJ kg⁻¹, about 15 MJ kg, about 16 MJ kg, about 17 MJ kg⁻¹, about 18 MJ kg⁻¹, about 19 MJ kg, about 20 MJ kg⁻¹, or about 21 MJ kg. For instance, in some embodiments, the energy value is from about 5,589 British thermal units per pound (BTU/lb) to about 9,450 BTU/lb. In some embodiments, the first energy value is about 5,500 BTU/lb, about 6,000 BTU/lb, is about 6,500 BTU/lb, about 7,000 BTU/lb, is about 7,500 BTU/lb, about 8,000 BTU/lb, is about 8,500 BTU/lb, about 9,000 BTU/lb, is about 9,500 BTU/lb, or about 10,000 BTU/lb. Additional details and information regarding the garden waste are found at Boldrin et al., 2010, “Seasonal Generation and Composition of Garden Waste in Aarhus (Denmark),” Waste Management, 30(4), pg. 551-557; Shi et al., 2013, “Garden Waste Biomass for Renewable and Sustainable Energy Production in China: Protentional, Challenges and Development,” Renewable and Sustainable Energy Reviews, 22, pg. 432-437, each of which is hereby incorporated by reference in its entirety for all purposes.

In some embodiments, the stream of unsorted MSW includes wood matter. In some embodiments this wood matter is plant matter, such as cellulose, hemicelluloses, lignin, or a combination thereof. For instance, in some embodiments, the wood waste includes one or more hardwoods, one or more soft woods, one or more wood fibers, or a combination thereof. As a non-limiting example, in some embodiments, the wood waste includes wood furniture and cabinets, pallets and containers, scrap lumber and pane, panels, engineered wood products, cardboard packaging, yard trimmings (e.g., leaves, grass clippings, brush, tree trimmings, removals, etc.), mulch, wood, or a combination thereof. In some embodiments, the wood waste further includes one or more additives (e.g., one or more adhesives, oner or more glues, one or more varnishes, one or more paints, etc.), one or more pollutants (e.g., one or more wood treatment products, one or more heavy metals), one or more contaminating materials (e.g., glass, plastics, metals, etc.), or a combination thereof. Additional details and information regarding the wood waste is found at Besserer et al., 2021, “Cascading Recycling of Wood Waste: A Review,” Polymers, 13(11), pg. 1752; Falk et al., 1997, “Opportunities for the Wood Waste Resource,” Forest Products Journal, 47(6), pg. 17-22; Faraca et al., 2019, “Resource Quality of Wood Waste: The Importance of Physical and Chemical Impurities in Wood Waste for Recycling,” Waste Management, 87, pg. 135-147, each of which is hereby incorporated by reference in its entirety for all purposes.

In some embodiments, the crop waste of the stream of unsorted MSW includes non-edible plant parts, such as those left in a field after a harvest. In some embodiments, the crop waste further includes crop-packing plants or that which is discarded during crop processing. For instance, in some embodiments, the crop waste one or more cereal crops, one or more grain crops, one or more legume crops, one or more oil crops, one or more fiber crops, one or more sugar crops, one or more tuber crops, one or more fruit crops, one or more vegetable crops, or a combination thereof. As a non-limiting example, in some embodiments, the crop waste includes apples, asparagus, barley, broccoli, brussels sprouts, carrots, cassava, chick peas, corn, cotton, eggplant, grapes, kale, lemons, lentils, lettuce, limes, millet, mustard, oats, okra, onions, orange, palm fruits, peaches, peanuts, pears, peas, potatoes, pulses, radishes, rapeseed, rice, rye, safflower, sesame, sorghum, soybean, strawberries, sugar beets, sugarcane, sunflower, sweet potatoes, tobacco, tomatoes, tree nuts, watermelons, wheat, wool, or a combination thereof. In some embodiments, a second energy value of the crop waste is from about 5 MJ kg to about 18 MJ kg, from about 6 MJ kg to about 17 MJ kg⁻¹, from about 7 MJ kg⁻¹ to about 16 MJ kg⁻¹, from about 8 MJ kg⁻¹ to about 15 MJ kg, from about 9 MJ kg to about 14 MJ kg, from about 10 MJ kg⁻¹ to about 13 MJ kg⁻¹, or from about 11 MJ kg⁻¹ to about 14 MJ kg⁻¹. In some embodiments, the second energy value of the crop waste is about 6 MJ kg, about 7 MJ kg⁻¹, about 8 MJ kg, about 9 MJ kg⁻¹, about 10 MJ kg, about 11 MJ kg⁻¹, about 12 MJ kg⁻¹, about 13 MJ kg, about 14 MJ kg⁻¹, about 15 MJ kg, about 16 MJ kg, or about 17 MJ kg⁻¹. For instance, in some embodiments, the second energy value of the crop waste is from about 2,150 BTU/lb to about 7,739 BTU/lb. In some embodiments, the second energy value of the crop waste is about 2,150 BTU/lb, about 2,500 BTU/lb, about 3,000 BTU/lb, about 3,500 BTU/lb, about 4,000 BTU/lb, about 4,500 BTU/lb, about 5,000 BTU/lb, about 5,500 BTU/lb, about 6,000 BTU/lb, about 6,500 BTU/lb, about 7,000 BTU/lb, about 7,500 BTU/lb, or about 8,000 BTU/lb. Additional details and information regarding the crop waste is found at Lal et al., 2005, “World Crop Residues Production and Implications of its Use as a Biofuel,” Environmental International, 31(4), pg. 575-584; Giombolini et al., 2011, “Testing the Local Reality: Does the Willamette Valley Growing Region Produce Enough to Meet the Needs of the Local Population? A Comparison of Agriculture Production and Recommended Dietary Requirements,” Agriculture and Human Values, 28(2), pg. 247-262, each of which is hereby incorporated by reference in its entirety for all purposes.

In some embodiments, the stream of unsorted MSW includes slaughterhouse waste, such as waste generated in farming and/or slaughtering of animals, such as poultry, cattle, sheep, pigs, or a combination thereof. For instance, in some embodiments, the slaughterhouse waste includes animal waste such as carcasses or parts of one or more animals, including products of animal origin not intended for direct human consumption. As a non-limiting example, in some embodiments, the slaughterhouse waste includes blood, bone, screenings, fat, oil, grease, settlings, sludge, floatation tailings, trimmings, feathers, feet, intestinal content, or a combination thereof. Additional details and information regarding the slaughterhouse waste are found at Salminen et al., 2002, “Anaerobic Digestion of Organic Solid Poultry Slaughterhouse Waste—A Review,” Bioresource Technology, 83(1), pg. 13-26; Adhikari et al., 2018, “Utilization of Slaughterhouse Waste in Value-Added Applications: Recent Advances in the Development of Wood Adhesives,” Polymers, 10(2), pg. 176, each of which is hereby incorporated by reference in its entirety for all purposes. Accordingly, the use of slaughterhouse waste, which has a high fat and/or protein content, provides for use as a suitable substrate for the anaerobic digestion process task 114.

Furthermore, in some embodiments the stream of unsorted MSW includes used food fat. In some embodiments, the used food fat includes one or more fat globules. For instance, in some embodiments, the used food fat includes one or more fatty acids, such as one or more saturated fatty acids, one or more mono-unsaturated fatty acids, one or more polyunsaturated fatty acids, or a combination thereof. In some embodiments, the used food fat includes a cholesterol. Additional details and information regarding suitable used food fat is found at Rohrmann et al., 2003, “Development and Validation of a Short Food List to Assess the Intake of Total Fat, Saturated, Mono-Unsaturated, Polyunsaturated Fatty Acids and Cholesterol,” The European Journal of Public Health, 13(3), pg. 262-268, which is hereby incorporated by reference in its entirety for all purposes.

In some embodiments, the stream of unsorted MSW includes grease. In some embodiments this grease is an oil, a thickening agent, an additive, or a combination thereof. For instance, in some embodiments, the grease includes a solid to semi-fluid material or dispersion of a thickening agent in a liquid lubricant. In some embodiments, the grease includes about 70 vol % to about 95 vol % oil, about 75 vol % to about 95 vol % oil, about 75 vol % to about 90 vol % oil, about 80 vol % to about 95 vol % oil, or about 80 vol % to about 90 vol % oil. Additional details and information regarding the grease is found at Lugt et al., 2009, “A Review on Grease Lubrication in Rolling Bearings,” Tribology Transactions, 52(4), 2009, pg. 470-480, which is hereby incorporated by reference in its entirety for all purposes.

In some embodiments, the stream of unsorted MSW includes manure. In some such embodiments the manure is animal dung and/or urine obtained from livestock. In some embodiments, the manure is substantially solid. In some embodiments, the manure includes carbon, nitrogen, water, free air space (FAS), or a combination thereof. In some embodiments, the manure includes a wood waste, such as sawdust or wood. In some embodiments, the manure has an organic matter content (OM) from about 53% to about 94%, from about 55% to about 90%, from about 60% to about 90%, from about 60% to about 85%, from about 65% to about 90%, from about 70% to about 85%, or from about 75% to about 90%. Additional details and information regarding the manure is found at Barrena et al., 2011, “Respirometric Screening of Several Types of Manure and Mixtures Intended for Composting,” Bioresource Technology, 102(2), pg. 1367-1377; Odedina et al., 2011, “Effect of Types of Manure on Growth and Yield of Cassava (Manihot Esculenta, Crantz),” Researcher, 3(5), pg. 1-8, each of which is hereby incorporated by reference in its entirety for all purposes.

In some embodiments, the stream of unsorted MSW includes sewage sludge in the form of a slurry.

In some embodiments, the stream of unsorted MSW 450 includes between 10% weight percent (wt %) and 30 wt % wood waste, between 11 wt % and 29 wt % wood waste, between 13 wt % and 25 wt % wood waste, between 15 wt % and 23 wt % wood waste, or between 17 wt % and 21 wt % wood waste. In some embodiments, the stream of unsorted MSW 450 includes between 8 wt % and 12 wt % wood waste, between 9 wt % and 13 wt % wood waste, between 10 wt % and 14 wt % wood waste, between 11 wt % and 15 wt % wood waste, between 12 wt % and 16 wt % wood waste, between 13 wt % and 17 wt % wood waste, between 14 wt % and 18 wt % wood waste, between 15 wt % and 19 wt % wood waste, between 16 wt % and 20 wt % wood waste, between 17 wt % and 21 wt % wood waste, between 18 wt % and 22 wt % wood waste, between 19 wt % and 23 wt % wood waste, between 20 wt % and 24 wt % wood waste, between 21 wt % and 25 wt % wood waste, between 22 wt % and 26 wt % wood waste, between 23 wt % and 27 wt % wood waste, between 24 wt % and 28 wt % wood waste, between 25 wt % and 29 wt % wood waste, between 26 wt % and 30 wt % wood waste, between 27 wt % and 31 wt % wood waste, or between 28 wt % and 32 wt % wood waste. In some embodiments, the stream of unsorted MSW 450 includes at least 8 wt % wood waste, at least 9 wt % wood waste, at least 10% wood waste, at least 11% wood waste, at least 12 wt % wood waste, at least 13 wt % wood waste, at least 14 wt % wood waste, at least 15 wt % wood waste, at least 16 wt % wood waste, at least 17 wt % wood waste, at least 18 wt % wood waste, at least 19 wt % wood waste, at least 20 wt % wood waste, at least 21 wt % wood waste, at least 22 wt % wood waste, at least 23 wt % wood waste, at least 24 wt % wood waste, at least 25 wt % wood waste, at least 26 wt % wood waste, at least 27 wt % wood waste, at least 28 wt % wood waste, at least 29 wt % wood waste, or at least 30 wt % wood waste. In some embodiments, the stream of unsorted MSW 450 includes at most 8 wt % wood waste, at most 9 wt % wood waste, at most 10 wt % wood waste, at most 11% wood waste, at most 12 wt % wood waste, at most 13 wt % wood waste, at most 14 wt % wood waste, at most 15 wt % wood waste, at most 16 wt % wood waste, at most 17 wt % wood waste, at most 18 wt % wood waste, at most 19 wt % wood waste, at most 20 wt % wood waste, at most 21 wt % wood waste, at most 22 wt % wood waste, at most 23 wt % wood waste, at most 24 wt % wood waste, at most 25 wt % wood waste, at most 26 wt % wood waste, at most 27 wt % wood waste, at most 28 wt % wood waste, at most 29 wt % wood waste, or at most 30 wt % wood waste.

In some embodiments, the stream of unsorted MSW 450 includes sulfur, nitrogen, nickel, vanadium, an asphaltene, one or more aromatics, or a combination thereof. In some embodiments the sulfur, nitrogen, nickel, vanadium, an asphaltene, or aromatic is present in an amount of less than one percent w/w, less than 0.1 percent w/w, or less than 0.1 percent w/w. In some embodiments the one or more aromatics is toluene, xylene, or 1,3,5-trimethylbenzene, either alone or in any combination. In some embodiments the one or more aromatics is benzene, ethylbenzene, toluene, p-xylene, o-xylene, styrene, naphthalene, propyl-benzene, 1,2-diethylbenzene, 1,3-diethylbenzene, 1,2,4-trimethylbenzene, 1,3,5-trimethylbenzene, 2-ethyltoluene, 3-ethyltoluene, either alone or in any combination.

Block 208. Referring to block 208, in some embodiments, the stream of unsorted MSW 450 consists of between 1,000 tons and 15,000 tons of material per day, between 1,500 tons and 14,500 tons of material per day, between 2,000 tons and 14,000 tons of material per day, between 2,500 tons and 13,500 tons of material per day, between 3,000 tons and 13,000 tons of material per day, between 3,500 tons and 12,500 tons of material per day, between 4,000 tons and 12,000 tons of material per day, between 4,500 tons and 11,500 tons of material per day, between 5,000 tons and 11,000 tons of material per day, between 5,500 tons and 10,500 tons of material per day, between 6,000 tons and 10,000 tons of material per day, between 6,500 tons and 9,500 tons of material per day, between 7,000 tons and 9,000 tons of material per day, or between 7,500 tons and 8,500 tons of material per day. In some embodiments, the stream of unsorted MSW consists of between 907,185 kilograms (kg) and 13.6 million kg of material per day, between 1.8 million kg and 10.9 million kg of material per day, or between 6.8 million kg and 7.7 million kg per day. In some embodiments, the stream of unsorted MSW consists of about 0.9 million kg of material per day, 1.5 million kg of material per day, 2.0 million kg of material per day, 2.5 million kg of material per day, 3.0 million kg of material per day, 3.5 million kg of material per day, 4.0 million kg of material per day, 4.5 million kg of material per day, 5.0 million kg of material per day, 5.5 million kg of material per day, 6.0 million kg of material per day, 6.5 million kg of material per day, 7.0 million kg of material per day, 7.5 million kg of material per day, 8.0 million kg of material per day, 8.5 million kg of material per day, 9.0 million kg of material per day, 9.5 million kg of material per day, 10.0 million kg of material per day, 10.5 million kg of material per day, 11.0 million kg of material per day, or 11.5 million kg of material per day.

Block 210. Referring to block 210, the method 200 includes processing, by one or more sorting mechanisms (e.g., one or more sorting mechanisms 402 of FIG. 4), the stream of unsorted MSW 450, which produces a stream of sorted MSW (e.g., stream of sorted MSW 452 of FIG. 4). Accordingly, the one or more sorting mechanisms is configured to take at least a portion of the stream of unsorted MSW from one or more sources as an input and produce a stream of sorted MSW. For instance, in some embodiments, the stream of unsorted MSW 450 includes a variety of materials such as metal, paper, plastic, glass, wood, or a combination thereof, which requires processing in order to obtain valuable materials utilized for producing a reformer feed gas and/or a methanol product (e.g., method 300 of FIGS. 3A through 3C). Accordingly, the one or more sorting mechanisms includes one or more instruments (e.g., instruments 110 of FIG. 1) that allow for the method 200 to uniformly segregate the variety of materials of the stream of unsorted MSW 450 in order to form the steam of sorted MSW 452.

Block 212. Referring to block 212, in some embodiments, the one or more sorting mechanisms 402 includes one or more mechanical sorting mechanisms (e.g., one or more mechanism instruments 110 of FIG. 1), one or more electromechanical sorting mechanisms (e.g., one or more electromechanical mechanism instruments 110 of FIG. 1), one or more manual sorting mechanisms, or a combination thereof. For instance, in some embodiments, the one or more mechanical sorting mechanisms include a non-sensor (e.g., sensor 182 of FIG. 1) based mechanisms, such as a wet and dry sorting mechanism.

In some embodiments, the one or more mechanical or electromechanical sorting mechanisms includes a hopper sorting mechanism, a bag opening sorting mechanism, a sizing filter mechanism, a shredding sorting mechanism, a trommel sorting mechanism, a three-dimensional sorting mechanism, an optical sorting mechanism, a ballistic sorting mechanism, a magnetic sorting mechanism, an eddy current sorting mechanism, a hydrothermal digestible material extraction sorting mechanism, or a combination thereof. For instance, in some embodiments the magnetic sorting mechanism 110 is utilized to separate ferrous material from the stream of unsorted MSW, such as by utilizing a magnetic flux to attract and hold the ferrous material in the stream of unsorted MSW to a revolving shell of the magnetic sorting mechanism 110, which produces the stream of sorted MSW that is substantially devoid of the ferrous material. In some embodiments, the shredding sorting mechanism is utilized to reduce a size of material in the stream of unsorted MSW to a maximum dimension, such as about 1 in any direction. Moreover, in some embodiments, bulk waste material included in the stream of unsorted MSW is pulverized by the one or more sorting mechanisms 110 into particles of uniform, or substantially uniform, size using forces produced by pressure, impact, cutting, abrasion, or a combination thereof during comminution, for convenient handling and/or to remove contaminants when processing the stream of unsorted MSW. As a non-limiting example, in some embodiments, the stream of unsorted is processed by one or more sorting mechanisms 110 including a first set of instruments 110 (e.g., a first screw press sorting mechanism 110-1, a second disc screen sorting mechanism 110-2, a third shredder sorting mechanism 110-3, a fourth magnetic sorting mechanism 110-4, or a combination thereof), such as in order to product dry waste from the stream of unsorted MSW. In some embodiments, the stream of unsorted MSW is processed by a second set of instruments 110 in the one or more sorting mechanisms 110 (e.g., a fifth comminution sorting mechanism 110-5, a sixth swing-hammer shredder sorting mechanism 110-6, a seventh rotating drum sorting mechanism 110-7, an eighth alligator shear sorting mechanism 110-8, a ninth hammer mill sorting mechanism 110-9, a tenth ring mill sorting mechanism 110-10, an eleventh impact crusher sorting mechanism 110-11, or a combination thereof).

In some embodiments, the one or more manual sorting mechanisms utilize a subject (e.g., personnel) to oversee removal of material from the stream of unsorted MSW. For instance, in some embodiments, a portion of the stream of unsorted MSW is examined by one or more subjects, which allows the one or more subjects to manually remove material from the stream of unsorted MSW. As a non-limiting example, in some embodiments, the one or more subjects examine the stream of unsorted MSW, identify an object in the stream of unsorted MSW, remove the object from the stream of unsorted MSW oversized, or a combination thereof. In some embodiments, the object is a prohibited object or an undesirable object, such as a non-biodegradable polymer. However, the present disclosure is not limited thereto.

As another non-limiting example, in some embodiments, the first set of instruments 110 of the one or more sorting mechanisms includes a bag opening sorting mechanism configured to expose an interior volume of a bag to expose contents therein included in the stream of unsorted MSW 450. Once the contents of the interior volume of the bag is exposed, a sizing sorting mechanism in the first set of instruments 110 of the one or more sorting mechanism 402 is utilized to shred material of the stream of unsorted MSW 450 in order to separate material by physical size, such as forming a first portion of the stream of unsorted MSW that includes material having a size greater than a first size (e.g., greater than 1 inch diameter) and a second portion of the stream of unsorted MSW 450 includes material having a second size different from the first size (e.g., less than or equal to 1 inch diameter). However, the present disclosure is not limited thereto. A portion of the stream of unsorted MSW 450 (e.g., the first portion) undergo further processing by one or more trommel sorting mechanisms, one or more optical sorting mechanisms, one or more ballistic sorting mechanisms, one or more magnetic sorting mechanisms in order to remove ferrous metals from the portion of the stream of unsorted MSW 450, one or more eddy current sorting mechanisms to remove aluminum from the portion of the stream of unsorted MSW 450, one or more hydrothermal digestible material extraction mechanism, or a combination thereof.

Additional details and information regarding the one or sorting mechanisms is found at Gundupalli et al., 2017, “A Review on Automated Sorting of Sourced-separated Municipal Solid Waste for Recycling,” Waste Management, 60, pg. 56-74.

In some embodiments, a first portion of the stream of unsorted MSW 450 separated from the stream of sorted MSW 452 (e.g., the unsorted MSW that is not organic) is provided to a remote facility, such as a landfill facility. However, the present disclosure is not limited thereto. For instance, in some embodiments, a second portion of the stream of unsorted MSW separated from the stream of sorted MSW includes ferrous metals. Furthermore, in some embodiments, a second portion of the stream of unsorted MSW 450 separated from the stream of sorted MSW 452 includes aluminum.

In some embodiments, the first set of instruments 110 in the one or more sorting mechanisms 402 is utilized to extract material including an organic fraction from the stream of unsorted MSW 450, such as to produce a first portion of the steam of sorted MSW 452. For instance, in some embodiments, the extracted material from the stream of unsorted MSW 450 includes the food waste, the garden waste, the wood waste, the crop waste, the food manufacture byproduct, the slaughterhouse waste, the used food fat, the oil, the grease, the manure, the biosolid, the sewage sludge, or the combination thereof included in the stream of unsorted MSW 450. However, the present disclosure is not limited thereto.

Accordingly, by processing the stream of unsorted MSW 450 using the one or more sorting mechanism 110, the stream of sorted MSW 452 is produced that does not include contaminants previously included in the stream of unsorted MSW 450. For instance, solid grit type material processed by the one or more sorting mechanisms 402 prevents such solid grit type material from being included in the stream of sorted MSW, which would otherwise settle in an anaerobic digestion apparatus (e.g., anaerobic digestion apparatus 406 of FIG. 4), and/or thin film plastic type materials that would float in a solution (e.g., solution of block 214 of FIG. 2A) and create a seal on an upper end portion of the anaerobic digestion, which blocks production of the reformer feed gas.

In some embodiments, a portion of the stream of unsorted MSW and/or the stream of sorted MSW (e.g., portion of stream of unsorted MSW 460 of FIG. 4) is subjected to an incineration process task (e.g., incineration process task 114-3 of FIG. 4). In some embodiments, the incineration process task 114 includes heating the portion of the stream of sorted MSW to produce a refuse-derived fuel (RDF) product and/or heat (e.g., product 462 of FIG. 4, product 476 of FIG. 4). However, the present disclosure is not limited thereto. In some embodiments, the incineration process task 114 produces fly ash, bottom ash, flue gases, particulates, or a combination thereof. Accordingly, in some embodiments, the portion of the stream of sorted MSW subjected to the incineration includes non-volatile material that is separated from the stream of the stream of sorted MSW by the sorting mechanism task 114-1. However, the present disclosure is not limited thereto. Additional details and information regarding the production of RDF is found at Kuen-Song et al., 1999, “Pyrolysis Kinetics of Refuse-derived Fuel,” Fuel Processing Technology, 60, pg. 103-110, which is hereby incorporated by reference in its entirety for all purposes.

In some embodiments, a rejection rate (e.g., a rate at which material is removed from the stream of unsorted MSW by the one or more sorting mechanism) is between about 5% and about 95%, about 10% and about 70%, about 15% and about 50%, or about 20% and about 30%. In some embodiments, the rejection rate is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In some embodiments, the rejection rate is at most 5%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95%.

In some embodiments, the one or more sorting mechanisms is configured to produce the stream of sorted MSW that is processed to ensure a uniform particle size. In some embodiments, the uniform particle size is between 50 mesh to 120, 55 mesh to 120, between 55 mesh to 115, between 60 mesh to 115 mesh, between 60 mesh to 100 mesh, or from 60 mesh to 90 mesh. In some embodiments, the uniform particle size at most 5 mesh, at most 10 mesh, at most 15 mesh, at most 20 mesh, at most 25 mesh, at most 30 mesh, at most 35 mesh, at most 40 mesh, at most 45 mesh, at most 50 mesh, at most 55 mesh, at most 60 mesh, at most 65 mesh, at most 70 mesh, at most 75 mesh, at most 80 mesh, at most 85 mesh, at most 90 mesh, or at most 95 mesh. In some embodiments, the uniform particle size at least 5 mesh, at least 10 mesh, at least 15 mesh, at least 20 mesh, at least 25 mesh, at least 30 mesh, at least 35 mesh, at least 40 mesh, at least 45 mesh, at least 50 mesh, at least 55 mesh, at least 60 mesh, at least 65 mesh, at least 70 mesh, at least 75 mesh, at least 80 mesh, at least 85 mesh, at least 90 mesh, or at least 95 mesh.

Block 214. Referring to block 214, the method 200 further includes retaining a solution in an anaerobic digestion apparatus (e.g., fourth task 114-4 of FIG. 4, anaerobic digestion instrument 110 of FIG. 1, etc.). Accordingly, the anaerobic digestion apparatus is configured to take at least a portion of the solution as an input and produce a reformer feed gas (e.g., reformer feed gas 466 of FIG. 4).

In some embodiments, anaerobic digestion is a complex task 114 that includes a hydrolysis phase, an acidogenesis phase, an acetogensis/dehydration task, a methanation task, or a combination thereof. In some embodiments, each respective phase of the anaerobic digestion task 114 is carried out by different consortia of microorganisms. For instance, in some embodiments, hydrolyzing and fermenting microorganisms are responsible for an initial interaction with biopolymers and monomers in the solution to produce acetate, hydrogen, and varying amounts of volatile fatty acids such as propionate and butyrate. For instance, in some embodiments, volatile fatty acids in the solution are converted into acetate and hydrogen by obligate hydrogen-producing acetogenic bacteria. In some embodiments, hydrolytic microorganisms excrete hydrolytic enzymes (e.g., cellulase, cellobiase, xylanase, amylase, lipase, and protease). In some embodiments, a complex consortium of microorganisms participates in the hydrolysis and fermentation of organic material in the solution.

Additional details and information regarding anaerobic digestion is found in Weiland et al., 2010, “Biogas Production: Current State and Perspectives,” Applied Microbiology and Biotechnology, 85(4), pg. 249-860; Zupančič et al., 2012, “Anaerobic Treatment and Biogas Production from Organic Waste,” Management of Organic Waste, 2, print, each of which is hereby incorporated by reference in its entirety for all purposes.

In some embodiments, the anaerobic digestion apparatus includes one or more anaerobic digestion reactor instruments. In some embodiments, the one or more anaerobic digestion reactor instruments 110 includes two or more anaerobic digestion reactor instruments connected in parallel fluidic communication, such that a fluid flow splits (e.g., bifurcates) among a first anaerobic digestion reactor instrument 110-1 and a second digestion reactor instrument 110-2 and eventually combine downstream into a single flow. In some embodiments, the one or more anaerobic digestion reactor instruments 110 includes two or more anaerobic digestion reactor instruments connected in series fluidic communication, such that a fluid flow flows from the first anaerobic digestion reactor instrument 110-1 to the second digestion reactor instrument 110-2 so that the flow is in a continuous line without any branching. In some embodiments, the anaerobic digestion reactor instrument is a single stage anaerobic digestion process.

Furthermore, in some embodiments, the anaerobic digestion apparatus includes a grit sweeper sorting mechanism, such as a first grit sweeper sorting mechanism configured to remove solid materials at a bottom end portion of the anaerobic digestion apparatus. Moreover, in some embodiments, the anaerobic digestion apparatus includes a thin film plastic sorting mechanism that is configured to remove material that floats at a surface (e.g., an upper surface, a liquid surface, etc.) of the anaerobic digestion apparatus. However, the present disclosure is not limited thereto.

Accordingly, in some embodiments, the solution includes a first portion of the stream of sorted MSW 452. For instance, in some embodiments, the first portion of the stream of sorted MSW 452 substantially includes digestible biological solids, such as the food waste, the garden waste, the wood waste, the crop waste, the food manufacture byproduct, the slaughterhouse waste, the used food fat, the used edible oil, the used grease from food (e.g., used food grease), the manure, the biosolid, the sewage sludge, or the combination thereof included in the stream of unsorted MSW 450 that is processed by the one or more sorting mechanisms (e.g., block 210 of FIG. 2A, block 212 of FIG. 2A, task 114-1 of FIG. 4, task 114-2 of FIG. 4, etc.). In some embodiments, the digestible biological solids include organic matter including: plant material, whether grown on land or water; animal products and manure; food processing and forestry by-products; urban wastes; or a combination thereof. In some embodiments, a first portion of the stream of sorted MSW 452 includes one or more complex biopolymers (e.g., polysaccharides, polypeptides, proteins, lipids, etc.), one or more monomers and/or one or more oligomers (e.g., sugar, amino acid, long chain fatty acids, etc.), one or more volatile fatty acids, or a combination thereof. Additional details and information regarding the use of digestible biological solids for anaerobic digestion is found at Fontana et al., 2018, “Microbial Activity Response to Hydrogen Injection in Thermophilic Anaerobic Digesters Revealed by Genomic-centric Metatranscriptomics,” Microbiome, 6(1), pg. 1-14, which is hereby incorporated by reference in its entirety for all purposes.

In some embodiments, a first portion of the stream of sorted MSW 452 includes a plurality of polymers where the respective polymers in the plurality of polymers do not all have the same molecular weight. In some such embodiments, the polymers in the plurality of polymers fall into a weight range with a corresponding distribution of chain lengths. In some embodiments, a polymer in the plurality of polymers is a branched polymer molecular system comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. pg. 6, which is hereby incorporated by reference herein in its entirety for all purposes.

In some embodiments, a first portion of the stream of sorted MSW 452 includes a plurality of polymers where the respective polymers in the plurality of polymers are polypeptides. As used herein, the term “polypeptide” means two or more amino acids or residues linked by a peptide bond. The terms “polypeptide” and “protein” are used interchangeably herein and include oligopeptides and peptides. An “amino acid,” “residue” or “peptide” refers to any of the twenty standard structural units of proteins as known in the art, which include imino acids, such as proline and hydroxyproline. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine are all considered amino acids. Other variants or analogs of the amino acids are known in the art. Thus, a polypeptide may include synthetic peptidomimetic structures such as peptoids. See Simon et al., 1992, Proceedings of the National Academy of Sciences USA, 89, 9367, which is hereby incorporated by reference herein in its entirety for all purposes. See also Chin et al., 2003, Science 301, 964; and Chin et al., 2003, Chemistry & Biology 10, 511, each of which is incorporated by reference herein in its entirety for all purposes.

In some embodiments, a first portion of the stream of sorted MSW 452 includes a plurality of polymers where the respective polymers in the plurality of polymers have any number of posttranslational modifications. Thus, a polymer includes those proteins that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are also encompassed by the present disclosure.

Accordingly, by retaining the solution in the anerobic digestion apparatus, a reformer feed gas (e.g., reformer feed gas 466 of FIG. 4) is produced, such as by utilizing a task 114 inducing anaerobic digestion of the solution in the anaerobic digestion apparatus. For instance, in some embodiments, the reformer feed gas includes from about 60 vol % to about 65 vol % methane (e.g., 62 vol %) and from about 38 vol % to about 40 vol % carbon dioxide. Moreover, in some embodiments, the reformer feed gas includes about 58 vol % to about 66 vol % methane, about 58 vol % to about 65 vol % methane, about 58 vol % to about 64 vol % methane, about 59 vol % to about 65 vol % methane, about 59 vol % to about 64 vol % methane, about 59 vol % to about 63 vol % methane, about 60 vol % to about 64 vol % methane, about 60 vol % to about 63 vol % methane, about 60 vol % to about 62 vol % methane, about 61 vol % to about 64 vol % methane, about 61 vol % to about 63 vol % methane, or about 61 vol % to about 62 vol % methane. In some embodiments, the reformer feed gas includes at least 58 vol % methane, at least 59 vol % methane, at least 60 vol % methane, at least 61 vol % methane, at least 62 vol % methane, at least 63 vol % methane, at least 64 vol % methane, or at least 65 vol % methane. In some embodiments, the reformer feed gas includes at most 58 vol % methane, at most 59 vol % methane, at most 60 vol % methane, at most 61 vol % methane, at most 62 vol % methane, at most 63 vol % methane, at most 64 vol % methane, or at most 65 vol % methane.

Furthermore, in some embodiments, the reformer feed gas includes from 35 vol % to 43 vol % carbon dioxide, from 35 vol % to 42 vol % carbon dioxide, from 35 vol % to 41 vol % carbon dioxide, from 36 vol % to 42 vol % carbon dioxide, from 36 vol % to 41 vol % carbon dioxide, from 36 vol % to 40 vol % carbon dioxide, from 37 vol % to 42 vol % carbon dioxide, from 37 vol % to 41 vol % carbon dioxide, from 37 vol % to 40 vol % carbon dioxide, from 37 vol % to 39 vol % carbon dioxide, from 38 vol % to 42 vol % carbon dioxide, from 38 vol % to 41 vol % carbon dioxide, from 38 vol % to 40 vol % carbon dioxide, from 38 vol % to 39 vol % carbon dioxide, from 39 vol % to 41 vol % carbon dioxide, or from 39 vol % to 40 vol % carbon dioxide. In some embodiments, the reformer feed gas includes at least 35 vol % carbon dioxide, at least 36 vol % carbon dioxide, at least 37 vol % carbon dioxide, at least 38 vol % carbon dioxide, at least 39 vol % carbon dioxide, at least 40 vol % carbon dioxide, at least 41 vol % carbon dioxide, or at least 42 vol % carbon dioxide. In some embodiments, the reformer feed gas includes at most 35 vol % carbon dioxide, at most 36 vol % carbon dioxide, at most 37 vol % carbon dioxide, at most 38 vol % carbon dioxide, at most 39 vol % carbon dioxide, at most 40 vol % carbon dioxide, at most 41 vol % carbon dioxide, or at most 42 vol % carbon dioxide.

From this, the method 200 produces the reformer feed gas 466 through anaerobic digestion of the solution in the anaerobic digestion apparatus. For instance, the solution includes bacteria of the first portion of the stream of sorted MSW 452 that interact with the digestible biological solids therein, producing the reformer feed gas 466, such as a stream of the reformer feed gas 466.

Block 216. Referring to block 216, in some embodiments, retaining the solution produces the reformer feed gas at a mass flow rate of between 6,000 kg/h and 15,000 kg/h, between 6,500 kg/h and 15,000 kg/h, between 7,000 kg/h and 14,500 kg/h, between 7,500 kg/h and 14,000 kg/h, between 8,000 kg/h and 13,500 kg/h, between 8,500 kg/h and 13,000 kg/h, between 9,000 kg/h and 12,500 kg/h, between 9,500 kg/h and 12,000 kg/h, between 10,000 kg/h and 14,500 kg/h, between 10,500 kg/h and 12,000 kg/h, between 6,200 kg/h and 9,800 kg/h, between 6,500 kg/h and 9,500 kg/h, between 6,700 kg/h and 9,300 kg/h, between 7,000 kg/h and 9,000 kg/h, between 7,200 kg/h and 8,800 kg/h, between 7,500 kg/h and 8,500 kg/h, or between 7,700 kg/h and 8,300 kg/h. In some embodiments, the mass flow rate of the produced reformer feed gas is at least 6,000 kg/h, at least 6,100 kg/h, at least 6,200 kg/h, at least 6,300 kg/h, at least 6,400 kg/h, at least 6,500 kg/h, at least 6,600 kg/h, at least 6,700 kg/h, at least 6,800 kg/h, at least 6,900 kg/h, at least 7,000 kg/h, at least 7,100 kg/h, at least 7,200 kg/h, at least 7,300 kg/h, at least 7,400 kg/h, at least 7,500 kg/h, at least 7,600 kg/h, at least 7,700 kg/h, at least 7,800 kg/h, at least 7,900 kg/h, at least 8,000 kg/h, at least 8,100 kg/h, at least 8,200 kg/h, at least 8,300 kg/h, at least 8,400 kg/h, at least 8,500 kg/h, at least 8,600 kg/h, at least 8,700 kg/h, at least 8,800 kg/h, at least 8,900 kg/h, at least 9,000 kg/h, at least 9,100 kg/h, at least 9,200 kg/h, at least 9,300 kg/h, at least 9,400 kg/h, at least 9,500 kg/h, at least 9,600 kg/h, at least 9,700 kg/h, at least 9,800 kg/h, at least 9,900 kg/h, at least 10,000 kg/h, at least at least 10,100 kg/h, at least 10,200 kg/h, at least 10,300 kg/h, at least 10,400 kg/h, at least 10,500 kg/h, at least 10,600 kg/h, at least 10,700 kg/h, at least 10,800 kg/h, at least 10,900 kg/h, at least 11,000 kg/h, at least at least 11,100 kg/h, at least 11,200 kg/h, at least 11,300 kg/h, at least 11,400 kg/h, at least 11,500 kg/h, at least 11,600 kg/h, at least 11,700 kg/h, at least 11,800 kg/h, at least 11,900 kg/h, at least 12,000 kg/h, at least at least 12,100 kg/h, at least 12,200 kg/h, at least 12,300 kg/h, at least 12,400 kg/h, at least 12,500 kg/h, at least 12,600 kg/h, at least 12,700 kg/h, at least 12,800 kg/h, at least 12,900 kg/h, at least 13,000 kg/h, at least at least 13,100 kg/h, at least 13,200 kg/h, at least 13,300 kg/h, at least 13,400 kg/h, at least 13,500 kg/h, at least 13,600 kg/h, at least 13,700 kg/h, at least 13,800 kg/h, at least 13,900 kg/h, at least 14,000 kg/h, at least at least 14,100 kg/h, at least 14,200 kg/h, at least 14,300 kg/h, at least 14,400 kg/h, at least 14,500 kg/h, at least 14,600 kg/h, at least 14,700 kg/h, at least 14,800 kg/h, at least 14,900 kg/h, or at least 15,000 kg/h. In some embodiments, the mass flow rate of the produced reformer feed gas is at most 6,000 kg/h, at most 6,100 kg/h, at most 6,200 kg/h, at most 6,300 kg/h, at most 6,400 kg/h, at most 6,500 kg/h, at most 6,600 kg/h, at most 6,700 kg/h, at most 6,800 kg/h, at most 6,900 kg/h, at most 7,000 kg/h, at most 7,100 kg/h, at most 7,200 kg/h, at most 7,300 kg/h, at most 7,400 kg/h, at most 7,500 kg/h, at most 7,600 kg/h, at most 7,700 kg/h, at most 7,800 kg/h, at most 7,900 kg/h, at most 8,000 kg/h, at most 8,100 kg/h, at most 8,200 kg/h, at most 8,300 kg/h, at most 8,400 kg/h, at most 8,500 kg/h, at most 8,600 kg/h, at most 8,700 kg/h, at most 8,800 kg/h, at most 8,900 kg/h, at most 9,000 kg/h, at most 9,100 kg/h, at most 9,200 kg/h, at most 9,300 kg/h, at most 9,400 kg/h, at most 9,500 kg/h, at most 9,600 kg/h, at most 9,700 kg/h, at most 9,800 kg/h, at most 9,900 kg/h, at most 10,000 kg/h, at most at most 10,100 kg/h, at most 10,200 kg/h, at most 10,300 kg/h, at most 10,400 kg/h, at most 10,500 kg/h, at most 10,600 kg/h, at most 10,700 kg/h, at most 10,800 kg/h, at most 10,900 kg/h, at most 11,000 kg/h, at most at most 11,100 kg/h, at most 11,200 kg/h, at most 11,300 kg/h, at most 11,400 kg/h, at most 11,500 kg/h, at most 11,600 kg/h, at most 11,700 kg/h, at most 11,800 kg/h, at most 11,900 kg/h, at most 12,000 kg/h, at most at most 12,100 kg/h, at most 12,200 kg/h, at most 12,300 kg/h, at most 12,400 kg/h, at most 12,500 kg/h, at most 12,600 kg/h, at most 12,700 kg/h, at most 12,800 kg/h, at most 12,900 kg/h, at most 13,000 kg/h, at most at most 13,100 kg/h, at most 13,200 kg/h, at most 13,300 kg/h, at most 13,400 kg/h, at most 13,500 kg/h, at most 13,600 kg/h, at most 13,700 kg/h, at most 13,800 kg/h, at most 13,900 kg/h, at most 14,000 kg/h, at most at most 14,100 kg/h, at most 14,200 kg/h, at most 14,300 kg/h, at most 14,400 kg/h, at most 14,500 kg/h, at most 14,600 kg/h, at most 14,700 kg/h, at most 14,800 kg/h, at most 14,900 kg/h, or at most 15,000 kg/h. For instance, in some embodiments, the mass flow rate of the reformer feed gas is between 13,200 pounds per hour (lb/hr) and 33,100 lb/hr, between 14,000 and 25,000 lb/hr, between 15,400 lb/hr and 19,850 lb/hr, or between 16,500 lb/hr and 18,750 lb/hr. In some embodiments, the mass flow rate of the reformer feed gas is at least 13,000 lb/hr, at least 13,500 lb/hr, at least 14,000 lb/hr, at least 14,500 lb/hr, at least 15,000 lb/hr, at least 15,500 lb/hr, at least 16,000 lb/hr, at least 16,500 lb/hr, at least 17,000 lb/hr, at least 17,500 lb/hr, at least 18,000 lb/hr, at least 18,500 lb/hr, at least 19,000 lb/hr, at least 19,500 lb/hr, at least 20,000 lb/hr, at least 20,500 lb/hr, at least 21,000 lb/hr, at least 21,500 lb/hr, at least 22,000 lb/hr, at least 22,500 lb/hr, at least 23,000 lb/hr, at least 23,500 lb/hr, at least 24,000 lb/hr, at least 24,500 lb/hr, at least 25,000 lb/hr, at least 25,500 lb/hr, at least 26,000 lb/hr, at least 26,500 lb/hr, at least 27,000 lb/hr, at least 27,500 lb/hr, at least 28,000 lb/hr, at least 28,500 lb/hr, at least 29,000 lb/hr, at least 29,500 lb/hr, at least 30,000 lb/hr, at least 30,500 lb/hr, at least 31,000 lb/hr, at least 31,500 lb/hr, at least 32,000 lb/hr, at least 32,500 lb/hr, at least 33,000 lb/hr, or at least 33,500 lb/hr. In some embodiments, the mass flow rate of the reformer feed gas is at most 13,000 lb/hr, at most 13,500 lb/hr, at most 14,000 lb/hr, at most 14,500 lb/hr, at most 15,000 lb/hr, at most 15,500 lb/hr, at most 16,000 lb/hr, at most 16,500 lb/hr, at most 17,000 lb/hr, at most 17,500 lb/hr, at most 18,000 lb/hr, at most 18,500 lb/hr, at most 19,000 lb/hr, at most 19,500 lb/hr, at most 20,000 lb/hr, at most 20,500 lb/hr, at most 21,000 lb/hr, at most 21,500 lb/hr, at most 22,000 lb/hr, at most 22,500 lb/hr, at most 23,000 lb/hr, at most 23,500 lb/hr, at most 24,000 lb/hr, at most 24,500 lb/hr, at most 25,000 lb/hr, at most 25,500 lb/hr, at most 26,000 lb/hr, at most 26,500 lb/hr, at most 27,000 lb/hr, at most 27,500 lb/hr, at most 28,000 lb/hr, at most 28,500 lb/hr, at most 29,000 lb/hr, at most 29,500 lb/hr, at most 30,000 lb/hr, at most 30,500 lb/hr, at most 31,000 lb/hr, at most 31,500 lb/hr, at most 32,000 lb/hr, at most 32,500 lb/hr, at most 33,000 lb/hr, or at most 33,500 lb/hr.

Block 218. Referring to block 218, in some embodiments, the first portion of the stream of sorted MSW 452 includes greater than or equal to 90 wt % biological waste, greater than or equal to 90.5 wt % biological waste, greater than or equal to 91 wt % biological waste, greater than or equal to 91.5 wt % biological waste, greater than or equal to 92 wt % biological waste, greater than or equal to 92.5 wt % biological waste, greater than or equal to 93 wt % biological waste, greater than or equal to 93.5 wt % biological waste, greater than or equal to 94 wt % biological waste, greater than or equal to 94.5 wt % biological waste, greater than or equal to 95 wt % biological waste, greater than or equal to 95.5 wt % biological waste, greater than or equal to 96 wt % biological waste, greater than or equal to 96.5 wt % biological waste, greater than or equal to 97 wt % biological waste, greater than or equal to 97.5 wt % biological waste, greater than or equal to 98 wt % biological waste, greater than or equal to 98.5 wt % biological waste, greater than or equal to 99 wt % biological waste, or greater than or equal to 99.5 wt % biological waste. In some embodiments, the first portion of the stream of sorted MSW 452 has between 90 wt % and 99. wt % biological waste, between 90 wt % and 99 wt % biological waste, between 90 wt % and 95 wt % biological waste, between 90 wt % and 94 wt % biological waste, between 92 wt % and 99 wt % biological waste, between 92 wt % and 97 wt % biological waste, between 92 wt % and 95 wt % biological waste, 95 wt % and 99.9 wt % biological waste, between 95.5 wt % and 99.5 wt % biological waste, between 96 wt % and 99 wt % biological waste, between 96.5 wt % and 98.5 wt % biological waste, or between 97 wt % and 98 wt % biological waste. Accordingly, in some such embodiments, the solution includes a substantial portion of biological waste, which is a substrate anaerobic digestion during performance of the anaerobic digestion task 114.

Block 220. Referring to block 220, in some embodiments, the anaerobic digestion apparatus includes a slurry stir mechanism configured to continuously or intermittently agitate the solution for the epoch. For instance, in some embodiments, an anaerobic digestion instrument 110 includes the slurry stir mechanism in order to form a continuously or intermittently stirred-stank reactor (CSTR) instrument. In some embodiments, the slurry stir mechanism includes a magnetic stirrer. However, the present disclosure is not limited thereto. In some embodiments, the slurry stir mechanism is mechanical, hydraulic, or pneumatic in order to bring the microorganisms in contact with the solution, to facilitate an up-flow of gaseous bubbles, to achieve constant temperature conditions in the solution, a uniform distribution of suspended and/or dissolved solids, or a combination thereof.

Block 222. Referring to block 222 of FIG. 2B, in some embodiments, the first portion of the stream of sorted MSW 452 is retained in the anaerobic digestion apparatus for an epoch, which allows for anaerobic digestion to occur. In some embodiments, the epoch is from about 5 days to about 60 days, from about 15 days to about 55 days, from about 20 days to about 60 days, from about 20 days to about 55 days, from about 30 days to about 60 days, from about 30 days to about 55 days, from about 30 days to about 50 days, from about 35 days to about 60 days, from about 35 days to 55 days, from about 35 days to about 50 days, or from about 40 days to about 55 days. In some embodiments, the epoch is at least 30 days, at least 31 days, at least 32 days, at least 33 days, at least 34 days, at least 35 days, at least 36 days, at least 37 days, at least 38 days, at least 39 days, 40 days, at least 41 days, at least 42 days, at least 43 days, at least 44 days, at least 45 days, at least 46 days, at least 47 days, at least 48 days, at least 49 days, 50 days, at least 51 days, at least 52 days, at least 53 days, at least 54 days, at least 55 days, at least 56 days, at least 57 days, at least 58 days, at least 59 days, or at least 60 days.

In some embodiments, the solution has a pH from about 6.0 to about 8.5, from about 6.5 to 8.5, from about 7.0 to about 8.5, from about 7.0 to about 8.0, or from about 7.5 to about 8.0. In some embodiments, the solution has a pH of at least 6.0, at least 6.5, at least 7.0, at least 7.5, at least 8.0, or at least 8.5. In some embodiments, the solution has a pH of at most 6.0, at most 6.5, at most 7.0, at most 7.5, at most 8.0, or at most 8.5.

In some embodiments, the solution including the first portion of the stream of sorted MSW 452 is, on a recurring basis, fed to the anaerobic digestion apparatus, such as once during an epoch, twice during an epoch, monthly during an epoch, weekly during an epoch, or daily during an epoch. In some embodiments, the solution including first portion of the stream of sorted MSW 452 is fed to the anaerobic digestion apparatus once per day, twice per day, three times per day, four times per day, five times per day, ten times per day, 24 times per day, 48 times per day, or about 72 times per day. In some embodiments, the recurring basis is a non-periodic basis. Furthermore, in some embodiments, the solution including first portion of the stream of sorted MSW 452 is fed on a continuous or intermittent basis to the anaerobic digestion apparatus.

Block 224. Referring to block 224, in some embodiments, during the retaining the solution, the solution is (e.g., maintained for an epoch) at a temperature of from about 80 degrees Fahrenheit (° F.) to about 110° F., from about 80° F. to about 105° F., from about 85° F. to about 110° F., from about 85° F. to about 105° F., from about 90° F. to about 110° F., from about 90° F. to about 105° F., from about 95° F. to about 105° F., or from about 100° F. to about 105° F. (e.g., from about 37.8° C. to about 40.6° C.), such as for a mesophilic anaerobic digestion task 114. In some embodiments, the temperature of the solution is during the mesophilic anaerobic digestion task 114 is at least 80° F., at least 81° F., at least 82° F., at least 83° F., at least 84° F., at least 85° F., at least 86° F., at least 87° F., at least 88° F., at least 89° F., at least 90° F., at least 91° F., at least 92° F., at least 93° F., at least 94° F., at least 95° F., at least 96° F., at least 97° F., at least 98° F., at least 99° F., at least 100° F., at least 101° F., at least 102° F., at least 103° F., at least 104° F., or at least 105° F. In some embodiments, the temperature of the solution is during the mesophilic anaerobic digestion task 114 is at most 80° F., at most 81° F., at most 82° F., at most 83° F., at most 84° F., at most 85° F., at most 86° F., at most 87° F., at most 88° F., at most 89° F., at most 90° F., at most 91° F., at most 92° F., at most 93° F., at most 94° F., at most 95° F., at most 96° F., at most 97° F., at most 98° F., at most 99° F., at most 100° F., at most 101° F., at most 102° F., at most 103° F., at most 104° F., or at most 105° F.

In some embodiments, the temperature of the solution is maintained at between 29° C. and 41° C., between 30° C. and 40° C., between 31° C. and 39° C., between 32° C. and 38° C., between 33° C. and 37° C., or between 34° C. and 36° C. In some embodiments, the temperature of the solution is at least 29° C., at least 30° C., at least 31° C., at least 32° C., at least 33° C., at least 34° C., at least 35° C., at least 36° C., at least 37° C., at least 38° C., at least 39° C., at least 40° C., or at least 41° C. In some embodiments, the temperature of the solution is at most 29° C., at most 30° C., at most 31° C., at most 32° C., at most 33° C., at most 34° C., at most 35° C., at most 36° C., at most 37° C., at most 38° C., at most 39° C., at most 40° C., or at most 41° C. In some embodiments, the mesophilic anaerobic digestion task 114 tolerates a fluctuations of +/−6° F.

In some embodiments, such as for a thermophilic anaerobic digestion task 114, the temperature of the solution is from about 115° F. to about 130° F., from about 115° F. to about 125° F., or from about 120° F. to about 125° F. In some embodiments, the temperature of the solution is during a thermophilic anaerobic digestion task 114 is at least 115° F., at least 116° F., at least 117° F., at least 118° F., at least 119° F., at least 120° F., at least 121° F., at least 122° F., at least 123° F., at least 124° F. at least 125° F., at least 126° F., at least 127° F., at least 128° F., at least 129° F., or at least 130° F. In some embodiments, the temperature of the solution is during a thermophilic anaerobic digestion task 114 is at most 115° F., at most 116° F., at most 117° F., at most 118° F., at most 119° F., at most 120° F., at most 121° F., at most 122° F., at most 123° F., at most 124° F. at most 125° F., at most 126° F., at most 127° F., at most 128° F., at most 129° F., or at most 130° F. In some embodiments, the temperature of the solution is maintained during the training at the temperature from about 46° C. to about 55° C., from about 48° C. to about 53° C., or from about 50° C. to about 54° C. In some embodiments, the temperature of the solution is maintained during the training at the temperature of at least 46° C., at least 47° C., at least 48° C., at least 49° C., at least 50° C., at least 51° C., at least 52° C., at least 53° C., at least 54° C., or at least 55° C. In some embodiments, the temperature of the solution is maintained during the training at the temperature of at most 46° C., at most 47° C., at most 48° C., at most 49° C., at most 50° C., at most 51° C., at most 52° C., at most 53° C., at most 54° C., or at most 55° C.

Additional details and information regarding the mesophilic anerobic digestion task 114 and thermophilic anaerobic digestion task 114 is found at Bassani et al., 2015, “Biogas Upgrading via Hydrogenotrophic Methanogenesis in Two-stage Continuous Stirred Tank Reactors at Mesophilic and Thermophilic Conditions,” Environmental Science and Technology, 49(20), pg. 12585-12593, which is hereby incorporated by reference in its entirety for all purposes.

Block 226. Referring to block 226, in some embodiments, the solution is formed (e.g., using a solution preparation task 114) by diluting the first portion of the stream of sorted MSW 452 with a fluid, such as with water (e.g., wastewater, spring water, deionized water, etc.). In some embodiments, the wt % concentration of the first portion of the stream of sorted MSW (e.g., volatile hydrocarbons) is from about 5 wt % to about 25 wt % solid concentration (e.g., solids in water), from 5 wt % to about 24 wt %, from 5 wt % to about 22 wt %, from 5 wt % to about 20 wt %, from 5 wt % to about 15 wt %, from about 5 wt % to about 16 wt % solid concentration, about 8 wt % to about 25 wt % solid concentration, from 8 wt % to about 24 wt %, from 8 wt % to about 22 wt %, from 8 wt % to about 20 wt %, from 8 wt % to about 18 wt %, from about 8 wt % to about 16 wt % solid concentration, from 10 wt % to about 24 wt %, from 10 wt % to about 22 wt %, from 10 wt % to about 20 wt %, from 10 wt % to about 18 wt %, from about 10 wt % to about 16 wt %, from about 10 wt % to about 14 wt %, from about 8 wt % to about 14 wt %, from about 7 wt % to about 12 wt %, or from about 8 wt % to about 11 wt % solid concentration. In some embodiments, the wt % solid concentration of the first portion of the stream of sorted MSW is from about 8 wt % to about 25 wt %, from about 10 wt % to about 16 wt %, or from about 8 wt % to about 10 wt % solid concentration. In some embodiments, the wt % solid concentration of the first portion of the stream of sorted MSW is at least 5 w %, at least 6 wt %, at least 7 wt %, at least 8 wt %, at least 9 wt %, at least 10 wt %, at least 11 wt %, at least 12 wt %, at least 13 wt %, at least 14 wt %, at least 15 wt %, at least 16 wt %, at least 17 wt %, at least 18 wt %, at least 19 wt %, at least 20 wt %, at least 21 wt %, at least 22 wt %, at least 23 wt %, at least 24 wt %, or at least 25 wt %. In some embodiments, the wt % solid concentration of the first portion of the stream of sorted MSW is at most 5 w %, at most 6 wt %, at most 7 wt %, at most 8 wt %, at most 9 wt %, at most 10 wt %, at most 11 wt %, at most 12 wt %, at most 13 wt %, at most 14 wt %, at most 15 wt %, at most 16 wt %, at most 17 wt %, at most 18 wt %, at most 19 wt %, at most 20 wt %, at most 21 wt %, at most 22 wt %, at most 23 wt %, at most 24 wt %, or at most 25 wt %. In some embodiments, decreasing the wt % solid concentration of the first portion of the stream of sorted MSW in the solution increases a required capacity of the anaerobic digestion apparatus. Moreover, decreasing the wt % solid concentration of the first portion of the stream of sorted MSW in the solution reduces the epoch for retaining the solution.

Block 228. Referring to block 228, in some embodiments, the solution includes a purge gas stream (e.g., purge gas stream 492 of FIG. 4) of a methanol synthesis reactor (e.g., methanol synthesis reactor task 114-9 of FIG. 4) that is recycled to the solution. For instance, in some embodiments, the purge gas stream 492 is utilized when passing a subset of a synthesis gas through a methanol synthesis reactor (e.g., block 348 of FIG. 3B) in order to mitigate an increase (e.g., build up) of one or more inert impurities, such as nitrogen, which add to operating (e.g., compression) costs.

In some embodiments, the purge gas stream has a mass flow rate in between 1,000 kg/hr and 2,000 kg/hr, between 1,100 kg/hr and 1,900 kg/hr, between 1,200 kg/hr and 1,800 kg/hr, between 1,300 kg/hr and 1,700 kg/hr, or between 1,400 kg/hr and 1,600 kg/hr. In some embodiments, the purge gas stream has a mass flow rate of at least 1,000 kg/hr, at least 1,100 kg/hr, at least 1,200 kg/hr, at least 1,300 kg/hr, at least 1,400 kg/hr, at least 1,500 kg/hr, at least 1,600 kg/hr, at least 1,700 kg/hr, at least 1,800 kg/hr, at least 1,900 kg/hr, or at least 2,000 kg/hr. In some embodiments, the purge gas stream has a mass flow rate of at most 1,000 kg/hr, at most 1,100 kg/hr, at most 1,200 kg/hr, at most 1,300 kg/hr, at most 1,400 kg/hr, at most 1,500 kg/hr, at most 1,600 kg/hr, at most 1,700 kg/hr, at most 1,800 kg/hr, at most 1,900 kg/hr, or at most 2,000 kg/hr. In some embodiments, the mass flow rate of the purge gas stream is between 2,200 lb/hr and 4,400 lb/hr, between 2,400 lb/hr and 4,200 lb/hr, between 2,200 lb/hr and 4,000 lb/hr, between 2,600 lb/hr and 3,800 lb/hr, between 2,800 lb/hr and 3,600 lb/hr, or between 3,000 lb/hr and 3,400 lb/hr. In some embodiments, the mass flow rate of the purge gas stream is at least 2,200 lb/hr, at least 2,300 lb/hr, at least 2,400 lb/hr, at least 2,500 lb/hr, at least 2,600 lb/hr, at least 2,700 lb/hr, at least 2,800 lb/hr, at least 2,900 lb/hr, at least 3,000 lb/hr, at least 3,100 lb/hr, at least 3,200 lb/hr, at least 3,300 lb/hr, at least 3,400 lb/hr, at least 3,500 lb/hr, at least 3,600 lb/hr, at least 3,700 lb/hr, at least 3,800 lb/hr, at least 3,900 lb/hr, at least 4,000 lb/hr, at least 4,100 lb/hr, at least, at least 4,200 lb/hr, at least 4,300 lb/hr, or at least 4,400 lb/hr. In some embodiments, the mass flow rate of the purge gas stream is at most 2,200 lb/hr, at most 2,300 lb/hr, at most 2,400 lb/hr, at most 2,500 lb/hr, at most 2,600 lb/hr, at most 2,700 lb/hr, at most 2,800 lb/hr, at most 2,900 lb/hr, at most 3,000 lb/hr, at most 3,100 lb/hr, at most 3,200 lb/hr, at most 3,300 lb/hr, at most 3,400 lb/hr, at most 3,500 lb/hr, at most 3,600 lb/hr, at most 3,700 lb/hr, at most 3,800 lb/hr, at most 3,900 lb/hr, at most 4,000 lb/hr, at most 4,100 lb/hr, at most, at most 4,200 lb/hr, at most 4,300 lb/hr, or at most 4,400 lb/hr.

In some embodiments, the purge gas stream 492 includes a hydrogen gas mixture. For instance, in some embodiments, the purge gas stream consists of between 3 vol % and 22 vol % hydrogen, between 4 vol % and 21 vol % hydrogen, between 5 vol % and 20 vol % hydrogen, between 6 vol % and 19 vol % hydrogen, between 7 vol % and 18 vol % hydrogen, between 8 vol % and 17 vol % hydrogen, between 9 vol % and 16 vol % hydrogen, between 10 vol % and 15 vol % hydrogen, between 11 vol % and 14 vol % hydrogen, or between 12 vol % and 13 vol % carbon. In some embodiments, the synthesis gas consists of less than 4 vol % hydrogen, less than 5 vol % hydrogen, less than 6 vol % hydrogen, less than 7 vol % hydrogen, less than 8 vol % hydrogen, less than 9 vol % hydrogen, less than 10 vol % hydrogen, less than 11 vol % hydrogen, less than 12 vol % hydrogen, less than 13 vol % hydrogen, less than 14 vol % hydrogen, less than 15 vol % hydrogen, less than 16 vol % hydrogen, less than 17 vol % hydrogen, less than 18 vol % hydrogen, less than 19 vol % hydrogen, or less than 20 vol % hydrogen. In some embodiments, the synthesis gas consists of more than 3 vol % hydrogen, more than 4 vol % hydrogen, more than 5 vol % hydrogen, more than 6 vol % hydrogen, more than 7 vol % hydrogen, more than 8 vol % hydrogen, more than 9 vol % hydrogen, more than 10 vol % hydrogen, more than 11 vol % hydrogen, more than 12 vol % hydrogen, more than 13 vol % hydrogen, more than 14 vol % hydrogen, more than 15 vol % hydrogen, more than 16 vol % hydrogen, more than 17 vol % hydrogen, more than 18 vol % hydrogen, more than 19 vol % hydrogen, or more than 20 vol % hydrogen.

Block 230. Referring to block 230, in some embodiments, the solution includes wastewater that is used to dilute the wt % concentration of the first portion of the stream of sorted MSW 452 (e.g., wastewater 470 of FIG. 4). For instance, in some embodiments, a methanol synthesis reactor task 114 (e.g., task 114-9 of FIG. 4, task 114-3 of FIG. 5, etc.) in fluidic communication with a methanol purification apparatus produces wastewater (e.g., wastewater 490 of FIG. 4), which is highly suitable to feed back into the anaerobic digestion apparatus. However, the present disclosure is not limited thereto. In some embodiments, the solution includes wastewater (e.g., wastewater 470 of FIG. 4), that is provided from a wastewater treatment task 114-5. For instance, in some embodiments, the wastewater 470 provided from the wastewater treatment task 114-5 includes a lipid feedstock. As a non-limiting example, in some embodiments, at least 10 weight % of the wastewater 470 is lipids, at least 15 weight % of the wastewater 470 is lipids, at least 20 weight % of the wastewater 470 is lipids, at least 25 weight % of the wastewater 470 is lipids, at least 30 weight % of the wastewater 470 is lipids, at least 35 weight % of the wastewater 470 is lipids, at least 40 weight % of the wastewater 470 is lipids, at least 45 weight % of the wastewater 470 is lipids, at least 50 weight % of the wastewater 470 is lipids, at least 55 weight % of the wastewater 470 is lipids, at least 60 weight % of the wastewater 470 is lipids, at least 65 weight % of the wastewater 470 is lipids, or at least 70 weight % of the wastewater 470 is lipids. In some embodiments, at most 25 weight % of the wastewater 470 is lipids, at most 30 weight % of the wastewater 470 is lipids, at most 35 weight % of the wastewater 470 is lipids, at most 40 weight % of the wastewater 470 is lipids, at most 45 weight % of the wastewater 470 is lipids, at most 50 weight % of the wastewater 470 is lipids, at most 55 weight % of the wastewater 470 is lipids, at most 60 weight % of the wastewater 470 is lipids, at most 65 weight % of the wastewater 470 is lipids, or at most 70 weight % of the wastewater 470 is lipids Accordingly, the wastewater 470 from the wastewater treatment task 114-5 provides a cheap, readily available, and abundant feedstock for the anaerobic digestion task 114-4.

Block 232. Referring to block 232, in some embodiments, the solution has a total volume of between 40,000 cubic meters (m³) and 100,000 m³, between 42,000 m³ and 98,000 m³, between 44,000 m³ and 96,000 m³, between 46,000 m³ and 94,000 m³, between 48,000 m³ and 92,000 m³, between 50,000 m³ and 90,000 m³, between 52,000 m³ and 88,000 m³, between 54,000 m³ and 86,000 m³, between 56,000 m³ and 84,000 m³, between 58,000 m³ and 82,000 m³, between 60,000 m³ and 80,000 m³, between 62,000 m³ and 78,000 m³, between 64,000 m³ and 76,000 m³, between 66,000 m³ and 74,000 m³, or between 68,000 m³ and 72,000 m³. In some embodiments, the total volume of the solution is at least 40,000 m³, at least 42,000 m³, at least 44,000 m³, at least 46,000 m³, at least 48,000 m³, at least 50,000 m³, at least 52,000 m³, at least 54,000 m³, at least 56,000 m³, at least 58,000 m³, at least 60,000 m³, at least 62,000 m³, at least 64,000 m³, at least 66,000 m³, at least 68,000 m³, at least 70,000 m³, at least 72,000 m³, at least 74,000 m³, at least 76,000 m³, at least 78,000 m³, at least 80,000 m³, at least 82,000 m³, at least 84,000 m³, at least 86,000 m³, at least 88,000 m³, at least 90,000 m³, at least 92,000 m³, at least 94,000 m³, at least 96,000 m³, at least 98,000 m³, or at least 100,000 m³. In some embodiments, the total volume of the solution is at most 40,000 m³, at most 42,000 m³, at most 44,000 m³, at most 46,000 m³, at most 48,000 m³, at most 50,000 m³, at most 52,000 m³, at most 54,000 m³, at most 56,000 m³, at most 58,000 m³, at most 60,000 m³, at most 62,000 m³, at most 64,000 m³, at most 66,000 m³, at most 68,000 m³, at most 70,000 m³, at most 72,000 m³, at most 74,000 m³, at most 76,000 m³, at most 78,000 m³, at most 80,000 m³, at most 82,000 m³, at most 84,000 m³, at most 86,000 m³, at most 88,000 m³, at most 90,000 m³, at most 92,000 m³, at most 94,000 m³, at most 96,000 m³, at most 98,000 m³, or at most 100,000 m³.

In some embodiments, the solution has a total volume between 1.4 million cubic feet (ft³) and 3.5 million ft³, between 1.5 million ft³ and 3.4 million ft³, between 1.6 million ft³ and 3.3 million ft³, between 1.7 million ft³ and 3.2 million ft³, between 1.8 million ft³ and 3.1 million ft³, between 1.9 million ft³ and 3 million ft³, between 2 million ft³ and 2.9 million ft³, between 2.1 million ft³ and 2.8 million ft³, between 2.2 million ft³ and 2.7 million ft³, between 2.3 million ft³ and 2.6 million ft³, or between 2.4 million ft³ and 2.5 million ft³. In some embodiments, the total volume of the solution is at least 1.4 million ft³, at least 1.5 million ft³, at least 1.6 million ft³, at least 1.7 million ft³, at least 1.8 million ft³, at least 1.9 million ft³, at least 2.0 million ft³, at least 2.1 million ft³, at least 2.2 million ft³, at least 2.4 million ft³, at least 2.5 million ft³, at least 2.6 million ft³, at least 2.7 million ft³, at least 2.8 million ft³, at least 2.9 million ft³, at least 3.0 million ft³, at least 3.1 million ft³, at least 3.2 million ft³, at least 3.3 million ft³, or at least 3.4 million ft³. In some embodiments, the total volume of the solution is at most 1.4 million ft³, at most 1.5 million ft³, at most 1.6 million ft³, at most 1.7 million ft³, at most 1.8 million ft³, at most 1.9 million ft³, at most 2.0 million ft³, at most 2.1 million ft³, at most 2.2 million ft³, at most 2.4 million ft³, at most 2.5 million ft³, at most 2.6 million ft³, at most 2.7 million ft³, at most 2.8 million ft³, at most 2.9 million ft³, at most 3.0 million ft³, at most 3.1 million ft³, at most 3.2 million ft³, at most 3.3 million ft³, or at most 3.4 million ft³.

In some embodiments, the total volume of the solution is dependent on a capacity (e.g., internal volume) of the anaerobic digestion apparatus.

Block 234. Referring to block 234, in some embodiments, the method 200 further includes treating the reformer feed gas 466, such as by a conversion process (e.g., conversion process task 114-7 of FIG. 4), which produces a treated reformer feed gas (e.g., stream 480 of FIG. 4). For instance, in some embodiments, the reformer feed gas 466 produced from anaerobic digestion apparatus includes a first portion of methane, a second portion of carbon dioxide, a third portion of water, and a fourth portion of impurities. In some embodiments, the fourth portion of impurities includes hydrogen sulfide, oxygen, nitrogen, or a combination thereof. However, the present disclosure is not limited thereto. This fourth portion of impurities is desirably removed the reformer feed gas 466 before the reformer feed gas 466 is provided to a methanol synthesis reactor task 114-9. For instance, if the impurities include sulfur and/or silicon materials, such as hydrogen sulfide and siloxanes, the sulfur and/or silicon act as poisons that deactivate one or more catalysts of the methanol synthesis reactor. Impurities such as oxygen deactivate reducing catalysts. Moreover, in some embodiments, the impurities such as water increases heating requirements and, therefore, energy consumption for producing the reformer feed gas and/or a methanol product. Furthermore, in some embodiments, impurities such as nitrogen require a purge of non-reacting compounds from the methanol synthesis reactor, which decrease yield of the methanol product.

In some embodiments, the solution is anaerobically digested when retained in an anaerobic digestion apparatus 110. In some embodiments, this anaerobically digestion (e.g., processing using an anaerobic digestion task 114) produces one or more gases such as methane, water (e.g., water vapor), hydrogen, carbon monoxide, or a combination thereof.

In some embodiments, the treating the reformer feed gas increases a ratio of methane to carbon dioxide in the reformer feed gas. In some embodiments, a first ratio of methane to carbon dioxide in the reformer feed gas before treating is between 2:1 and 3:1. For instance, in some embodiments, before treating, the reformer feed gas has a first ratio of methane to carbon dioxide of 3:2 (e.g., 60 vol % methane, 40 vol % carbon dioxide). In some embodiments, the first ratio of methane to carbon dioxide in the reformer feed gas before treating is at least 2:1, at least 3:2, or at least 3:1. In some embodiments, the first ratio of methane to carbon dioxide in the reformer feed gas before treating is at most 2:1, at most 3:2, or at most 3:1. Moreover, in some embodiments, the treated reformer feed gas has a second ratio of methane to carbon dioxide of 3:1 (e.g., about 75 vol % methane, about 25 vol % carbon dioxide). Moreover, in some embodiments, the treated reformer feed gas has a second ratio of methane to carbon dioxide of 4:1 (e.g., about 80 vol % methane, about 20 vol % carbon dioxide). In some embodiments, the second ratio of methane to carbon dioxide in the reformer feed gas after treating is at least 3:2, at least 3:1, or at least 4:1. In some embodiments, the second ratio of methane to carbon dioxide in the reformer feed gas after treating is at most 3:2, at most 3:1, or at most 4:1. However, the present disclosure is not limited thereto.

In some embodiments, a first carbon dioxide content of the reformer feed gas 466 is between 35 vol % and 45 vol %, and a second carbon dioxide content of the treated reformer feed gas is between 20 vol % and 23 vol %. For instance, in some embodiments, the first carbon dioxide content of the reformer feed gas is between 34 vol % and 46 vol %, between 34 vol % and 45 vol %, between 35$ and 44 vol %, between 35 vol % and 46 vol %, between 35 vol % and 45 vol %, between 35 vol % and 44 vol %, between 35 vol % and 43 vol %, between 36 vol % and 45 vol %, between 36 vol % and 54 vol %, between 36 vol % and 43 vol %, between 37 vol % and 44 vol %, between 37 vol % and 43 vol %, between 37 vol % and 42 vol %, between 38 vol % and 44 vol %, between 38 vol % and 43 vol %, between 38 vol % and 42 vol %, between 38 vol % and 41 vol %, between 39 vol % and 43 vol %, between 39 vol % and 42 vol %, or between 39 vol % and 40 vol %. In some embodiments, the first carbon dioxide content of the reformer feed gas is at least 33 vol %, at least 34 vol %, at least 35 vol %, at least 36 vol %, at least 37 vol %, at least 38 vol %, at least 39 vol %, at least 40 vol %, at least 41 vol %, at least 42 vol %, at least 43 vol %, at least 44 vol %, at least 45 vol %, or at least 46 vol %. In some embodiments, the first carbon dioxide content of the reformer feed gas is at most 33 vol %, at most 34 vol %, at most 35 vol %, at most 36 vol %, at most 37 vol %, at most 38 vol %, at most 39 vol %, at most 40 vol %, at most 41 vol %, at most 42 vol %, at most 43 vol %, at most 44 vol %, at most 45 vol %, or at most 46 vol %.

Moreover, in some embodiments, the second carbon dioxide content of the treated reformer feed gas is between 19 vol % and 24 vol %, between 19 vol % and 23 vol %, between 19 vol % and 22 vol %, between 20 vol % and 24 vol %, between 20 vol % and 23 vol %, between 20 vol % and 22 vol %, between 20 vol % and 21 vol %, between 21 vol % and 24 vol %, between 21 vol % and 23 vol %, between 21 vol % and 22 vol %, between 22 vol % and 24 vol %, or between 23 vol % and 24 vol %. In some embodiments, the second carbon dioxide content of the treated reformer feed gas is at least 19 vol %, at least 20 vol %, at least 21 vol %, at least 22 vol %, at least 23 vol %, at least 24 vol %, or at least 25 vol %. In some embodiments, the second carbon dioxide content of the treated reformer feed gas is at most 19 vol %, at most 20 vol %, at most 21 vol %, at most 22 vol %, at most 23 vol %, at most 24 vol %, or at most 25 vol %.

In some embodiments, the conversion process includes removing a first portion of the reformer feed gas that is nitrogen, a second portion of the reformer feed gas that is oxygen, a third portion of the reformer feed gas that is one or more silicon compounds, a fourth portion of the reformer feed gas that is sulfur or one or more sulfur compounds, or a combination thereof. For instance, in some embodiments, an oxygen removal apparatus removes oxygen from the reformer feed gas prior to providing the reformer feed gas to the methane reformer.

In some embodiments, the conversion process includes one or more dehydration processes, one or more fixed media bed processes, one or more chilling processes, one or more gas compression processes, or a combination thereof. In some embodiments, the conversion process includes one or more dehydration processes (e.g., parallel and/or series dehydration processes), one or more fixed media bed processes to remove sulfur compounds, a chilling process followed by a further dehydration process, a further fixed media bed process to remove siloxanes, one or more gas compression processes, or a combination thereof.

Now that methods 200 for producing a reformer feed gas have been described in accordance with various embodiments of the present disclosures, details regarding some processes in accordance with FIGS. 3A, 3B, and 3C will be described. Specifically, FIGS. 3A, 3B, and 3C collectively illustrates a flow chart of methods (e.g., method 300) for producing methanol, in accordance with embodiments of the present disclosure.

Various modules in the memory 192 of the computer system 100 perform certain processes of the methods 300 described in FIGS. 3A, 3B, and 3C, unless expressly stated otherwise. Furthermore, it will be appreciated that the processes in FIGS. 3A, 3B, and 3C can be encoded in a single module or any combination of modules.

Block 302. Referring to block 302 of FIG. 3A, a method 300 for producing methanol is provided.

In some embodiments, the method 300 is conducted at a computer system (e.g., distributed system 100 of FIG. 1, system 400 of FIG. 4, system 500 of FIG. 5 etc.). The computer system 100 includes one or more processors (e.g., CPU 172 of FIG. 1), and a memory (e.g., memory 192 of FIG. 1) that is coupled to the one or more processors 172. The memory 192 includes one or more programs (e.g., control module 106 of FIG. 1, client application 118 of FIG. 1, etc.) that is configured to be executed by the one or more processors 172. In other words, in some embodiments, the method 300 cannot be mentally performed because the computational complexity addressed by the method 300 requires use of the computer system.

Block 304. Referring to block 304, the method 300 includes providing a reformer feed gas (e.g., reformer feed gas 466 of FIG. 4, treated reformer feed gas 480 of FIG. 4, reformer feed gas 488 of FIG. 4, reformer feed gas 584 of FIG. 5) that includes methane and carbon dioxide to a methane reformer (e.g., task 114-9 of FIG. 4, task 114-2 of FIG. 5, etc.). From this, the method 300 produces a synthesis gas using the methane reformer. Accordingly, the methane reformer is configured to take at least a portion of the reformer feed gas (e.g., block 214 of FIG. 2A, block 234 of FIG. 2B, etc.) as an input and produce the synthesis gas.

In some embodiments, the methane is present in the reformer feed gas in an amount of between 20 vol % and 90 vol %. For instance, in some embodiments, the methane is present in the reformer feed gas in an amount of between 20 vol % and 90 vol % methane, between 20 vol % and 85 vol % methane, between 25 vol % and 90 vol % methane, between 25 vol % and 85 vol % methane, between 30 vol % and 80 vol % methane, between 30 vol % and 75 vol % methane, between 35 vol % and 80 vol % methane, between 35 vol % and 85 vol % methane, between 40 vol % and 90 vol %, between 40 vol % and 85 vol %, between 40 vol % and 80 vol % methane, between 40 vol % and 75 vol % methane, between 45 vol % and 90 vol % methane, between 45 vol % and 85 vol % methane, between 45 vol % and 80 vol % methane, between 45 vol % and 75 vol % methane, between 50 vol % and 70 vol % methane, between 50 vol % and 65 vol % methane, between 55 vol % and 70 vol % methane, or between 55 vol % and 65 vol % methane. In some embodiments, the reformer feed gas includes less than 90 vol % methane, less than 85 vol % methane, less than 80 vol % methane, less than 75 vol % methane, less than 70 vol % methane, less than 65 vol % methane, less than 60 vol % methane, less than 55 vol % methane, less than 50 vol % methane, less than 45 vol % methane, less than 40 vol % methane, less than 35 vol % methane, less than 30 vol % methane, less than 25 vol % methane, or less than 20 vol % methane. In some embodiments, the reformer feed gas includes more than 85 vol % methane, more than 80 vol % methane, more than 75 vol % methane, more than 70 vol % methane, more than 65 vol % methane, more than 60 vol % methane, more than 55 vol % methane, more than 50 vol % methane, more than 45 vol % methane, more than 40 vol % methane, more than 35 vol % methane, more than 30 vol % methane, more than 25 vol % methane, or more than 20 vol % methane.

Moreover, the carbon dioxide is present in the reformer feed gas in an amount of between 5 vol % and 80 vol %, between 5 vol % and 75 vol %, between 5 vol % and 70 vol %, between 5 vol % and 65 vol %, between 5 vol % and 60 vol %, between 5 vol % and 55 vol %, between 10 vol % and 65 vol %, between 10 vol % and 60 vol %, between 10 vol % and 55 vol %, between 10 vol % and 50 vol %, between 15 vol % and 60 vol %, between 15 vol % and 55 vol %, between 15 vol % and 50 vol %, between 15 vol % and 45 vol %, between 20 vol % and 55 vol %, between 20 vol % and 50 vol %, between 20 vol % and 45 vol %, between 30 vol % and 50 vol %, between 30 vol % and 45 vol %, between 30 vol % and 40 vol %, between 35 vol % and 55 vol %, between 40 vol % and 50 vol %, or between 45 vol % and 50 vol %. In some embodiments, the carbon dioxide is present in the reformer feed gas in an amount of less than 5 vol %, less than 7.5 vol %, less than 10 vol %, less than 12.5 vol %, less than 15 vol %, less than 17.5 vol %, less than 20 vol %, less than 22.5 vol %, less than 25 vol %, less than 27.5 vol %, less than 30 vol %, less than 32.5 vol %, less than 35 vol %, less than 37.5 vol %, less than 40 vol %, less than 42.5 vol %, less than 45 vol %, less than 47.5 vol %, less than 50 vol %, less than 52.5 vol %, less than 55 vol %, less than 57.5 vol %, less than 60 vol %, less than 62.5 vol %, less than 65 vol %, less than 67.5 vol %, less than 70 vol %, less than 72.5 vol %, less than 75 vol %, less than 77.5 vol %, or less than 80 vol %. In some embodiments, the carbon dioxide is present in the reformer feed gas in an amount of more than 5 vol %, more than 7.5 vol %, more than 10 vol %, more than 12.5 vol %, more than 15 vol %, more than 17.5 vol %, more than 20 vol %, more than 22.5 vol %, more than 25 vol %, more than 27.5 vol %, more than 30 vol %, more than 32.5 vol %, more than 35 vol %, more than 37.5 vol %, more than 40 vol %, more than 42.5 vol %, more than 45 vol %, more than 47.5 vol %, more than 50 vol %, more than 52.5 vol %, more than 55 vol %, more than 57.5 vol %, more than 60 vol %, more than 62.5 vol %, more than 65 vol %, more than 65 vol %, more than 67.5 vol %, more than 70 vol %, more than 72.5 vol %, more than 75 vol %, more than 77.5 vol %, or more than 80 vol %.

In some embodiments, the synthesis gas includes a first portion of hydrogen and a second portion of carbon monoxide. In some embodiments, the synthesis gas includes the first portion of hydrogen and a third portion of carbon dioxide. In some embodiments, the synthesis gas includes the first portion of hydrogen, the second portion of carbon monoxide, and the third portion of carbon dioxide. For instance, in some embodiments, a composition of the synthesis gas is characterized by a stoichiometric number S that is defined by a ratio of a difference of moles of hydrogen and carbon dioxide and a summation of the moles of carbon dioxide and carbon monoxide. In some embodiments, the stoichiometric number S of the synthesis gas is between 1 to 3.5, between 1.5 to 3, between 2 to 3, between 2.2 to 2.8, or between 2.4 to 2.6. In some embodiments, the stoichiometric number S of the synthesis gas is at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.1, at least 3.2, at least 3.3, or at least 3.4. In some embodiments, the stoichiometric number S of the synthesis gas is at most 1.8, at most 1.9, at most 2.0, at most 2.1, at most 2.2, at most 2.3, at most 2.4, at most 2.5, at most 2.6, at most 2.7, at most 2.8, at most 2.9, at most 3.0, at most 3.1, at most 3.2, at most 3.3, or at most 3.4. However, the present disclosure is not limited thereto. Accordingly, the stoichiometric value S relates to the presence of carbon dioxide that consumes the hydrogen via a reverse water-gas shift reaction. Additional details and information regarding the synthesis gas is found at Bozzano et al., 2016, “Efficient Methanol Synthesis: Perspectives, Technologies and Optimization Strategies,” Progress in Energy and Combustion Science, 56, pg. 71-105, which is hereby incorporated by reference in its entirety for all purposes.

Furthermore, in some embodiments, the synthesis gas includes the first portion of hydrogen, the second portion of carbon monoxide, the third portion of carbon dioxide, and a fourth portion of impurities. As a non-limiting example, in some embodiments, the fourth portion of impurities includes water (e.g., water vapor), sulfur, nitrogen, carbon dioxide, methane, one or more hydrocarbons, one or more acid gases, one or more particulates, or a combination thereof. In some embodiments, the fourth portion is present in the reformer feed gas in an amount between 20 vol % and 30 vol %, between 21 vol % and 29 vol %, between 22 vol % and 28 vol %, between 23 vol % and 27 vol %, or between 24 vol % and 26 vol %. In some embodiments, the fourth portion is present in the reformer feed gas in an amount of at least 20 vol %, at least 21 vol %, at least 22 vol %, at least 23 vol %, at least 24 vol %, at least 25 vol %, at least 26 vol %, at least 27 vol %, at least 28 vol %, at least 29 vol %, or at least 30 vol %. In some embodiments, the fourth portion is present in the reformer feed gas in an amount of at most 20 vol %, at most 21 vol %, at most 22 vol %, at most 23 vol %, at most 24 vol %, at most 25 vol %, at most 26 vol %, at most 27 vol %, at most 28 vol %, at most 29 vol %, or at most 30 vol %. As a non-limiting example, in some embodiments, the reformer feed gas includes about 25 vol % water vapor.

Block 306. Referring to block 306, in some embodiments, the reformer feed gas 466 further includes a third portion that is not reactive during the using the methanol synthesis reactor.

In some embodiments, the third portion includes a silicone compound, sulfur, a sulfur compound, or a combination thereof. For instance, in some embodiments, by producing the synthesis gas by inputting the reformer feed gas to the methane reformer yields lower concentration of impurities, such as sulfur, one or more halogenated compounds, one or more metals, or a combination thereof, which eases downstream purification tasks 114 and mitigates catalyst-poisoning issues found in the methanol synthesis reactor.

In some embodiments, the reformer feed gas 466 is a biogas. In some embodiments, the biogas includes methane and carbon dioxide. In some embodiments, the biogas includes hydrogen sulfide, ammonia, water vapor, or a combination thereof.

Block 308. Referring to block 308, in some embodiments, a first portion of the reformer feed gas includes a first gas mixture obtained from a landfill gas service provider. For instance, in some embodiments, the first gas mixture includes gas mixture that is obtained as a result from subjecting a wood waste to a pyrolysis process task 114. However, the present disclosure is not limited thereto.

Block 310. Referring to block 310, in some embodiments, the reformer feed gas includes a second gas mixture obtained from a municipal solid waste (MSW). In some embodiments, the MSW includes a food waste, a garden waste, a wood waste, a crop waste, a food manufacture byproduct, a slaughterhouse waste, a used food fat, a used edible oil, a used grease from food, a manure, a biosolid, a sewage sludge, or a combination thereof. In some embodiments, the MSW includes a hydrophobic and/or lipophilic compound. In some embodiments, the MSW is in situ.

In some embodiments, the providing the reformer feed gas 466 further includes utilizing a first portion of the MSW as an energy source for the methane reformer.

Block 312. Referring to block 312, in some embodiments, prior to the utilizing the first portion, the first portion of the MSW (e.g., first portion 458 of FIG. 4) is subjected to a pyrolysis process task (e.g., task 114-6 of FIG. 4). For instance, in some embodiments, the pyrolysis process task 114 includes heating organic portions of the MSW, so that thermally unstable compounds are broken down and evaporate with other volatile components. In some embodiments, these volatile components form a gaseous product (e.g., pyrolysis gas) includes tar, methane, aromatic hydrocarbons, steam, carbon dioxide, or a combination thereof. For instance, in some embodiments, at temperatures above 248° F. (e.g., 120° C.), organic materials begin to undergo some thermal decomposition, losing chemically bound moisture. As a non-limiting example, in some embodiments, hemicelluloses are degraded at 392° F. to 500° F. (e.g., 200° C. to 260° C.); cellulose is degraded at 464° F. to 662° F. (e.g., 240° C. to 350° C.); and lignin degraded at 536° F. to 932° F. (e.g., 280° C. to 500° C.) Moreover, in some embodiments, a solid product from pyrolysis process task 114 includes coke or char (e.g., residual carbon). In some embodiments, this solid product is then burned or used for gasification. However, the present disclosure in not limited thereto. In some embodiments, the first portion of the MSW 458 includes one or more impurities, such as lead, mercury, an herbicide, a pesticide, or the like, which are removed when subjected to the pyrolysis process. However, the present disclosure is not limited thereto.

In some embodiments, the first portion of the MSW includes the wood waste. In some embodiments, a percent moisture of first portion of the MSW (e.g., of the wood waste) is in between 80% and 15%, between 40% and 15%, between 35% and 16%, between 30% and 17%, or between 20% and 15%. In some embodiments, the percent moisture of first portion of the MSW is less than 25%, less than 24%, less than 23%, less than 22%, less than 21%, less than 20%, less than 19%, less than 18%, less than 17%, less than 16%, or less than 15%. In some embodiments, the percent moisture of first portion of the MSW is more than 24%, more than 23%, more than 22%, more than 21%, more than 20%, more than 19%, more than 18%, more than 17%, more than 16%, or more than 15%. For instance, in some embodiments, the pyrolysis process task 114-6 is conducted in a vacuum (e.g., an absence of air), which allows for thermally decomposing the wood waste in the first portion of the MSW. In some embodiments, the first portion of the MSW includes between 80% and 21% moisture, which must be reduced by a heating task 114 (e.g., a dehydrating task) to between 20% and 15% before being subjected to the pyrolysis process task 114-5. As a non-limiting example, in some embodiments, a spent digestate (e.g., digestate 468 of FIG. 4, digestate 474 of FIG. 4, etc.) from an anaerobic digestion process task 114 (e.g., block 214 of FIG. 2A, anaerobic digestion process task 114-5 of FIG. 4, etc.) that includes about 70 percent moisture, which must be dried to about 20 percent moisture before the pyrolysis process task 114-6. However, the present disclosure is not limited thereto. For instance, in some embodiments, the spent digestate includes a suspension of solids (e.g., plant fiber solids) in water, which is formed by subjecting the spent digestate to a filter press sorting mechanism, which provides a solution that is high in chemical oxygen demand.

In some embodiments, the pyrolysis process is conducted a temperature of from about 1,000° F. to about 1,800° F., from about 1,100° F. to about 1,750° F., about 1,200° F. to about 1,725° F., about 1,300° F. to about 1,700° F., from about 1,400° F. to about 1,650° F., about 1,500° F. to about 1,625° F., from about 1,025° F. to about 1,275° F., from about 1,050° F. to about 1,250° F., from about 1,075° F. to about 1,225° F., from about 1,100° F. to about 1,200° F., or from about 1,125° F. to about 1,175° F. In some embodiments, the pyrolysis process is conducted a temperature of less than 1,000° F., less than 1,025° F., less than 1,050° F., less than 1,075° F., less than 1,100° F., less than 1,125° F., less than 1,150° F., less than 1,175° F., less than 1,200° F., less than 1,225° F., less than 1,250° F., less than 1,275° F., less than 1,300° F., less than 1,325° F., less than 1,350° F., less than 1,375° F., less than 1,400° F., less than 1,425° F., less than 1,450° F., less than 1,475° F., less than 1,500° F., less than 1,525° F., less than 1,550° F., less than 1,575° F., less than 1,600° F., less than 1,625° F., less than 1,650° F., less than 1,675° F., less than 1,700° F., less than 1,725° F., less than 1,750° F., or less than 1,775° F. In some embodiments, the pyrolysis process is conducted a temperature of more than 1,000° F., more than 1,025° F., more than 1,050° F., more than 1,075° F., more than 1,100° F., more than 1,125° F., more than 1,150° F., more than 1,175° F., more than 1,200° F., more than 1,225° F., more than 1,250° F., more than 1,275° F., more than 1,300° F., more than 1,325° F., more than 1,350° F., more than 1,375° F., more than 1,400° F., more than 1,425° F., more than 1,450° F., more than 1,475° F., more than 1,500° F., more than 1,525° F., more than 1,550° F., more than 1,575° F., more than 1,600° F., more than 1,625° F., more than 1,650° F., more than 1,675° F., more than 1,700° F., more than 1,725° F., more than 1,750° F., or more than 1,775° F. For instance, in some embodiments, such as from about 1,000° F. to about 1,150° F., torrefaction occurs where the first portion of the MSW is heated to lightly brown a surface of the first portion of the MSW, as opposed to turning the surface into charcoal, such as in order to remove chemically bound water within the first portion of the MSW. At the high temperature, such as from about 1,150° F. to about 1,300° F., the first portion of the MSW is turned into charcoal. However, the present disclosure is not limited thereto. In some embodiments, the pyrolysis process is conducted a temperature of less than 525° C., less than 550° C., less than 575° C., less than 600° C., less than 625° C., less than 650° C., less than 675° C., less than 700° C., or less than 725° C., less than 750° C., less than 775° C., less than 800° C., or less than 825° C., less than 850° C., less than 875° C., less than 900° C., or less than 925° C., or less than 950° C. In some embodiments, the pyrolysis process is conducted a temperature of more than 525° C., more than 550° C., more than 575° C., more than 600° C., more than 625° C., more than 650° C., more than 675° C., more than 700° C., more than 725° C., more than 750° C., more than 775° C., more than 800° C., or more than 825° C., more than 850° C., more than 875° C., more than 900° C., or more than 925° C., or more than 950° C.

Accordingly, by including the wood waste in the first portion of the MSW and subjecting this to the pyrolysis process task 114-5, the method 300 decomposes cellulose, hemicellulose, lignin, or a combination thereof in the wood waste. In this way, the cellulose, hemicellulose, lignin, or the combination includes a polymer of sugars, such as a linear polysaccharide polymer with a plurality of glucose monosaccharide units. Accordingly, the hydroxy (OH) groups are chemically bound water that, when heated, reacts with hydrogen to provide water vapor and graphite or charcoal. However, the present disclosure is not limited thereto.

Block 314. Referring to block 314, in some embodiments the providing the reformer feed gas further includes utilizing a landfill gas stream as the energy source for the methane reformer. In some embodiments, the landfill gas stream is an uncleaned landfill gas stream. For instance, in some embodiments, a first landfill gas stream has between 20 vol % and 90 vol % methane, between 20 vol % and 85 vol % methane, between 25 vol % and 90 vol % methane, between 25 vol % and 85 vol % methane, between 30 vol % and 80 vol % methane, between 30 vol % and 75 vol % methane, between 35 vol % and 80 vol % methane, between 35 vol % and 85 vol % methane, between 40 vol % and 90 vol % methane, between 40 vol % and 85 vol % methane, between 40 vol % and 80 vol % methane, between 40 vol % and 75 vol % methane, between 45 vol % and 90 vol % methane, between 45 vol % and 85 vol % methane, between 45 vol % and 80 vol % methane, between 45 vol % and 75 vol % methane, between 50 vol % and 70 vol % methane, between 50 vol % and 65 vol % methane, between 55 vol % and 70 vol % methane, or between 55 vol % and 65 vol % methane. In some embodiments, the first landfill gas stream includes less than 90 vol % methane, less than 85 vol % methane, less than 80 vol % methane, less than 75 vol % methane, less than 70 vol % methane, less than 65 vol % methane, less than 60 vol % methane, less than 55 vol % methane, less than 50 vol % methane, less than 45 vol % methane, less than 40 vol % methane, less than 35 vol % methane, less than 30 vol % methane, less than 25 vol % methane, or less than 20 vol % methane. In some embodiments, the first landfill gas stream includes more than 85 vol % methane, more than 80 vol % methane, more than 75 vol % methane, more than 70 vol % methane, more than 65 vol % methane, more than 60 vol % methane, more than 55 vol % methane, more than 50 vol % methane, more than 45 vol % methane, more than 40 vol % methane, more than 35 vol % methane, more than 30 vol % methane, more than 25 vol % methane, or more than 20 vol % methane.

In some embodiments, the reformer feed gas includes a landfill gas stream, such as the first landfill gas stream (e.g., landfill gas stream 486 and/or reformer feed gas 488 of FIG. 4). For instance, in some embodiments, the reformer feed gas (e.g., reformer feed gas 584 of FIG. 5) is sourced from a landfill gas stream (e.g., landfill gas stream 582 of FIG. 5). In some embodiments, the reformer feed gas is sourced from an in situ landfill gas stream (e.g., stream 486 of Figure $). In some embodiments, the landfill gas steam is processed (e.g., by a sorting mechanism task 114-1 of FIG. 5), such as in order to remove one or more impurities, such as nitrogen, silica, or one or more metals (e.g., stream 486 of FIG. 4 is processed by task 114-8 of FIG. 4 to produce stream 488 of FIG. 4). Accordingly, this landfill gas stream is cleaned of contaminants by the one or more sorting mechanism and further processed to a methane reformer, a methanol synthesis reactor, and/or one or more purification instruments.

Block 316. Referring to block 316, in some embodiments, the method 300 further includes adjusting the reformer feed gas to have between 40 vol % and 85 vol % methane prior to the providing the reformer feed gas to the methane reformer. In some embodiments, the reformer feed gas is adjusted to have between 50 vol % and 70 vol % methane prior to the providing the reformer feed gas to the methane reformer. In some embodiments, the reformer feed gas is adjusted to adjusted to have 50 vol % or greater methane, 55 vol % or greater methane, 60 vol % or greater methane, 65 vol % or greater methane, 70 vol % or greater methane, 75 vol % or greater methane, 80 vol % or greater methane, or 85 vol % or greater methane prior to the providing the reformer feed gas to the methane reformer. In some embodiments, the reformer feed gas is adjusted to have 55 vol % or less methane, 60 vol % or less methane, 65 vol % or less methane, 70 vol % or less methane, 75 vol % or less methane, 80 vol % or less methane, or 85 vol % or less methane prior to the providing the reformer feed gas to the methane reformer.

In some embodiments, the adjusting is conducted by a pressure swing adsorption task 114, a cryogenic distillation task 114, a membrane fractionation task 114, a permeation task 114, or a combination thereof. For instance, in some embodiments, the adjusting is utilized to remove a respective portion of the reformer feed gas, such as a first portion of the reformer feed gas having a first density greater than a second portion of the reformer feed gas having a second density, such as separating one or more fluids and/or one or more solids. As a non-limiting example, in some embodiments, the adjusting utilizes a separation instrument including one or more distillation column instruments, one or more membrane fractionation instruments, one or more ion exchange adsorption instruments, one or more thermal adsorption instruments, one or more pressure swing adsorption instruments, one or more molecular sieve instruments, one or more flash drum instruments, one or more absorption column instruments, one or more adsorption column instruments, one or more wet scrubber instruments, one or more Venturi scrubber instruments, one or more centrifuge instruments, one or more chromatograph instruments, one or more crystallizer instruments, or a combination.

In some embodiments, the adjusting is conducted in accordance with a conversion process task (e.g., block 234 of FIG. 2B). However, the present disclosure is not limited thereto.

Block 318. Referring to block 318, in some embodiments, the method 300 further includes adjusting the reformer feed gas to have between 15 vol % and 50 vol % carbon dioxide prior to the providing the reformer feed gas to the methane reformer. In some embodiments, the reformer feed gas is adjusted to have between 15 vol % and 50 vol % carbon dioxide, between 15 vol % and 49 vol % carbon dioxide, between 15 vol % and 48 vol % carbon dioxide, between 16 vol % and 50 vol % carbon dioxide, between 16 vol % and 49 vol % carbon dioxide, between 16 vol % and 48 vol % carbon dioxide, between 17 vol % and 49 vol % carbon dioxide, between 17 vol % and 47 vol % carbon dioxide, between, between 20 vol % and 50 vol % carbon dioxide, between 20 vol % and 45 vol % carbon dioxide, between 20 vol % and 40 vol % carbon dioxide, between 22 vol % and 50 vol % carbon dioxide, between 22 vol % and 45 vol % carbon dioxide, between 22 vol % and 40 vol % carbon dioxide, between 25 vol % and 50 vol % carbon dioxide, between 25 vol % and 45 vol % carbon dioxide, between 25 vol % and 40 vol % carbon dioxide, between 28 vol % and 50 vol % carbon dioxide, between 28 vol % and 45 vol % carbon dioxide, between 28 vol % and 40 vol % carbon dioxide, between 30 vol % and 49 vol % carbon dioxide, between 30 vol % and 48 vol % carbon dioxide, between 31 vol % and 50 vol % carbon dioxide, between 31 vol % and 49 vol % carbon dioxide, between 31 vol % and 48 vol % carbon dioxide, between 32 vol % and 49 vol % carbon dioxide, between 32 vol % and 48 vol % carbon dioxide, between 32 vol % and 47 vol % carbon dioxide, between 33 vol % and 48 vol % carbon dioxide, between 33 vol % and 47 vol % carbon dioxide, between 33 vol % and 46 vol % carbon dioxide, between 34 vol % and 47 vol % carbon dioxide, between 34 vol % and 46 vol % carbon dioxide, between 35 vol % and 45 vol % carbon dioxide, between 35 vol % and 46 vol % carbon dioxide, between 35 vol % and 44 vol % carbon dioxide, between 36 vol % and 45 vol % carbon dioxide, between 36 vol % and 44 vol % carbon dioxide, between 36 vol % and 43 vol % carbon dioxide, between 37 vol % and 44 vol % carbon dioxide, between 37 vol % and 43 vol % carbon dioxide, between 37 vol % and 42 vol % carbon dioxide, between 37 vol % and 41 vol % carbon dioxide, between 37 vol % and 40 vol % carbon dioxide, between 37 vol % and 39 vol % carbon dioxide, between 38 vol % and 43 vol % carbon dioxide, between 38 vol % and 42 vol % carbon dioxide, between 38 vol % and 41 vol % carbon dioxide, between 38 vol % to 40 vol % carbon dioxide, between 38 vol % and 39 vol % carbon dioxide, or between 39 vol % and 40 vol % carbon dioxide prior to the providing to the methane reformer. In some embodiments, the reformer feed gas is adjusted to less than 15 vol % carbon dioxide, less than 16 vol % carbon dioxide, less than 17 vol % carbon dioxide, less than 18 vol % carbon dioxide, less than 19 vol % carbon dioxide, less than 20 vol % carbon dioxide, less than 21 vol % carbon dioxide, less than 22 vol % carbon dioxide, less than 23 vol % carbon dioxide, less than 24 vol % carbon dioxide, less than 25 vol % carbon dioxide, less than 26 vol % carbon dioxide, less than 27 vol % carbon dioxide, less than 28 vol % carbon dioxide, less than 29 vol % carbon dioxide, less than 30 vol % carbon dioxide, less than 31 vol % carbon dioxide, less than 32 vol % carbon dioxide, less than 33 vol % carbon dioxide, less than 34 vol % carbon dioxide, less than 35 vol % carbon dioxide, less than 36 vol % carbon dioxide, less than 37 vol % carbon dioxide, less than 38 vol % carbon dioxide, less than 39 vol % carbon dioxide, less than 40 vol % carbon dioxide, less than 41 vol % carbon dioxide, less than 42 vol % carbon dioxide, less than 43 vol % carbon dioxide, less than 44 vol % carbon dioxide, less than 45 vol % carbon dioxide, less than 46 vol % carbon dioxide, less than 47 vol % carbon dioxide, less than 48 vol % carbon dioxide, less than 49 vol % carbon dioxide, or less than 50 vol % carbon dioxide. In some embodiments, the reformer feed gas is adjusted to more than 15 vol % carbon dioxide, more than 16 vol % carbon dioxide, more than 17 vol % carbon dioxide, more than 18 vol % carbon dioxide, more than 19 vol % carbon dioxide, more than 20 vol % carbon dioxide, more than 21 vol % carbon dioxide, more than 22 vol % carbon dioxide, more than 23 vol % carbon dioxide, more than 24 vol % carbon dioxide, more than 25 vol % carbon dioxide, more than 26 vol % carbon dioxide, more than 27 vol % carbon dioxide, more than 28 vol % carbon dioxide, more than 29 vol % carbon dioxide, more than 30 vol % carbon dioxide, more than 31 vol % carbon dioxide, more than 32 vol % carbon dioxide, more than 33 vol % carbon dioxide, more than 34 vol % carbon dioxide, more than 35 vol % carbon dioxide, more than 36 vol % carbon dioxide, more than 37 vol % carbon dioxide, more than 38 vol % carbon dioxide, more than 39 vol % carbon dioxide, more than 40 vol % carbon dioxide, more than 41 vol % carbon dioxide, more than 42 vol % carbon dioxide, more than 43 vol % carbon dioxide, more than 44 vol % carbon dioxide, more than 45 vol % carbon dioxide, more than 46 vol % carbon dioxide, more than 47 vol % carbon dioxide, more than 48 vol % carbon dioxide, more than 49 vol % carbon dioxide, or more than 50 vol % carbon dioxide.

In some embodiments, the feedstock includes from about 55 vol % to about 65 vol % methane (e.g., 62 vol %) and from about 35 vol % to about 45 vol % carbon dioxide. For instance, in some embodiments, the feedstock includes about 58 vol % to about 66 vol % methane, about 58 vol % to about 65 vol % methane, about 58 vol % to about 64 vol % methane, about 59 vol % to about 65 vol % methane, about 59 vol % to about 64 vol % methane, about 59 vol % to about 63 vol % methane, about 60 vol % to about 64 vol % methane, about 60 vol % to about 63 vol % methane, about 60 vol % to about 62 vol % methane, about 61 vol % to about 64 vol % methane, about 61 vol % to about 63 vol % methane, or about 61 vol % to about 62 vol % methane. In some embodiments, the feedstock includes less than 58 vol % methane, less than 59 vol % methane, less than 60 vol % methane, less than 61 vol % methane, less than 62 vol % methane, less than 63 vol % methane, less than 64 vol % methane, or less than 65 vol % methane. In some embodiments, the feedstock includes more than 58 vol % methane, more than 59 vol % methane, more than 60 vol % methane, more than 61 vol % methane, more than 62 vol % methane, more than 63 vol % methane, more than 64 vol % methane, or more than 65 vol % methane.

Furthermore, in some embodiments, the feedstock includes from 35 vol % to 43 vol % carbon dioxide, from 35 vol % to 42 vol % carbon dioxide, from 35 vol % to 41 vol % carbon dioxide, from 36 vol % to 42 vol % carbon dioxide, from 36 vol % to 41 vol % carbon dioxide, from 36 vol % to 40 vol % carbon dioxide, from 37 vol % to 42 vol % carbon dioxide, from 37 vol % to 41 vol % carbon dioxide, from 37 vol % to 40 vol % carbon dioxide, from 37 vol % to 39 vol % carbon dioxide, from 38 vol % to 42 vol % carbon dioxide, from 38 vol % to 41 vol % carbon dioxide, from 38 vol % to 40 vol % carbon dioxide, from 38 vol % to 39 vol % carbon dioxide, from 39 vol % to 41 vol % carbon dioxide, or from 39 vol % to 40 vol % carbon dioxide. In some embodiments, the feedstock includes less than 35 vol % carbon dioxide, less than 36 vol % carbon dioxide, less than 37 vol % carbon dioxide, less than 38 vol % carbon dioxide, less than 39 vol % carbon dioxide, less than 40 vol % carbon dioxide, less than 41 vol % carbon dioxide, or less than 42 vol % carbon dioxide. In some embodiments, the feedstock includes more than 35 vol % carbon dioxide, more than 36 vol % carbon dioxide, more than 37 vol % carbon dioxide, more than 38 vol % carbon dioxide, more than 39 vol % carbon dioxide, more than 40 vol % carbon dioxide, more than 41 vol % carbon dioxide, or more than 42 vol % carbon dioxide.

Block 320. Referring to block 320, in some embodiments, the reformer feed gas consists of less than 5 vol % fossil natural gas. In some embodiments, the reformer feed gas consists of less than 4.5 vol % fossil natural gas. In some embodiments, the reformer feed gas consists of less than 4 vol % fossil natural gas. In some embodiments, the reformer feed gas consists of less than 3.5 vol % fossil natural gas. In some embodiments, the reformer feed gas consists of less than 3 vol % fossil natural gas. In some embodiments, the reformer feed gas consists of less than 2.5 vol % fossil natural gas. In some embodiments, the reformer feed gas consists of less than 2 vol % fossil natural gas. In some embodiments, the reformer feed gas consists of less than 1.5 vol % fossil natural gas. In some embodiments, the reformer feed gas consists of less than 1 vol % fossil natural gas. In some embodiments, the reformer feed gas consists of less than 0.5 vol % fossil natural gas. In some embodiments, the reformer feed gas consists of less than 0.1 vol % fossil natural gas.

Block 322. Referring to block 322, in some embodiments, the reformer feed gas consists of between 65 vol % and 90 vol % methane, between 65 vol % and 85 vol % methane, between 70 vol % and 90 vol % methane, between 70 vol % and 85 vol % methane, between 75 vol % and 80 vol % methane, between 75 vol % and 85 vol % methane, between 75 vol % and 90 vol % methane, between 70 vol % and 80 vol % methane, between 75 vol % and 85 vol % methane, between 75 vol % and 80 vol % methane, between 80 vol % and 85 vol % methane, or between 80 vol % and 90 vol % methane. In some embodiments, the reformer feed gas consists of less than 65 vol % methane, less than 66 vol % methane, less than 67 vol % methane, less than 68 vol % methane, less than 69 vol % methane, less than 70 vol % methane, less than 71 vol % methane, less than 72 vol % methane, less than 73 vol % methane, less than 74 vol % methane, less than 75 vol % methane, less than 76 vol % methane, less than 77 vol % methane, less than 78 vol % methane, less than 79 vol % methane, less than 80 vol % methane, less than 81 vol % methane, less than 82 vol % methane, less than 83 vol % methane, less than 84 vol % methane, less than 85 vol % methane, less than 86 vol % methane, less than 87 vol % methane, less than 88 vol % methane, less than 89 vol % methane, or less than 90 vol % methane. In some embodiments, the reformer feed gas consists of more than 65 vol % methane, more than 66 vol % methane, more than 67 vol % methane, more than 68 vol % methane, more than 69 vol % methane, more than 70 vol % methane, more than 71 vol % methane, more than 72 vol % methane, more than 73 vol % methane, more than 74 vol % methane, more than 75 vol % methane, more than 76 vol % methane, more than 77 vol % methane, more than 78 vol % methane, more than 79 vol % methane, more than 80 vol % methane, more than 81 vol % methane, more than 82 vol % methane, more than 83 vol % methane, more than 84 vol % methane, more than 85 vol % methane, more than 86 vol % methane, more than 87 vol % methane, more than 88 vol % methane, or more than 89 vol % methane.

Block 324. Referring to block 324, in some embodiments, the reformer feed gas during the providing the reformer feed gas to the methane reformer has a first mass flow rate between 6,000 kilograms per hour (kg/h) and 15,000 kg/h, between 6,250 kg/h and 14,750 kg/h, between 6,500 kg/h and 15,500 kg/h, between 6,750 kg/h and 14,250 kg/h, between 7,000 kg/h and 14,000 kg/h, between 6,250 kg/h and 13,750 kg/h, between 6,500 kg/h and 13,500 kg/h, between 6,750 kg/h and 13,250 kg/h, between 7,000 kg/h and 13,000 kg/h, between 7,250 kg/h and 12,750 kg/h, between 7,500 kg/h and 12,500 kg/h, between 7,750 kg/h and 12,250 kg/h, between 8,000 kg/h and 12,000 kg/h, between 8,250 kg/h and 11,750 kg/h, between 8,500 kg/h and 11,500 kg/h, between 8,750 kg/h and 11,250 kg/h, between 9,000 kg/h and 10,000 kg/h, between 9,250 kg/h and 10,750 kg/h, between 9,500 kg/h and 10,500 kg/h, or between 9,750 kg/h and 10,250 kg/h. In some embodiments, the first mass flow rate of the reformer feed gas is at least 6,000 kg/h, at least 6,250 kg/h, at least 6,500 kg/h, at least 6,750 kg/h, at least 7,000 kg/h, at least 7,250 kg/h, at least 7,500 kg/h, at least 7,750 kg/h, at least 8,000 kg/h, at least 8,250 kg/h, at least 8,500 kg/h, at least 8,750 kg/h, at least 9,000 kg/h, at least 9,250 kg/h, at least 9,500 kg/h, at least 9,750 kg/h, at least 10,000 kg/h, at least 10,250 kg/h, at least 10,500 kg/h, at least 10,750 kg/h, at least 11,000 kg/h, at least 11,250 kg/h, at least 11,500 kg/h, at least 11,750 kg/h, at least 12,000 kg/h, at least 12,250 kg/h, at least 12,500 kg/h, at least 12,750 kg/h, at least 13,000 kg/h, at least 13,250 kg/h, at least 13,500 kg/h, at least 13,750 kg/h, at least 14,000 kg/h, at least 14,250 kg/h, at least 14,500 kg/h, at least 14,750 kg/h, or at least 15,000 kg/h. In some embodiments, the first mass flow rate of the reformer feed gas is at most 6,000 kg/h, at most 6,250 kg/h, at most 6,500 kg/h, at most 6,750 kg/h, at most 7,000 kg/h, at most 7,250 kg/h, at most 7,500 kg/h, at most 7,750 kg/h, at most 8,000 kg/h, at most 8,250 kg/h, at most 8,500 kg/h, at most 8,750 kg/h, at most 9,000 kg/h, at most 9,250 kg/h, at most 9,500 kg/h, at most 9,750 kg/h, at most 10,000 kg/h, at most 10,250 kg/h, at most 10,500 kg/h, at most 10,750 kg/h, at most 11,000 kg/h, at most 11,250 kg/h, at most 11,500 kg/h, at most 11,750 kg/h, at most 12,000 kg/h, at most 12,250 kg/h, at most 12,500 kg/h, at most 12,750 kg/h, at most 13,000 kg/h, at most 13,250 kg/h, at most 13,500 kg/h, at most 13,750 kg/h, at most 14,000 kg/h, at most 14,250 kg/h, at most 14,500 kg/h, at most 14,750 kg/h, or at most 15,000 kg/h. In some embodiments, the first mass flow rate is between 13,000 lb/hr and 33,000 lb/hr, between 14,000 lb/hr and 32,000 lb/hr, between 15,000 lb/hr and 31,000 lb/hr, between 16,000 lb/hr and 30,000 lb/hr, between 19,000 lb/hr and 29,000 lb/hr, between 20,000 lb/hr and 28,000 lb/hr, between 21,000 lb/hr and 27,000 lb/hr, between 22,000 lb/hr and 26,000 lb/hr, or between 23,000 lb/hr and 25,000 lb/hr. In some embodiments, the first mass flow rate is at least 13,000 lb/hr, at least 14,000 lb/hr, at least 15,000 lb/hr, at least 16,000 lb/hr, at least 17,000 lb/hr, at least 18,000 lb/hr, at least 19,000 lb/hr, at least 20,000 lb/hr, at least 21,000 lb/hr, at least 22,000 lb/hr, at least 23,000 lb/hr, at least 24,000 lb/hr, at least 25,000 lb/hr, at least 26,000 lb/hr, at least 27,000 lb/hr, at least 28,000 lb/hr, at least 29,000 lb/hr, at least 30,000 lb/hr, at least 31,000 lb/hr, at least 32,000 lb/hr, or at least 33,000 lb/hr. In some embodiments, the first mass flow rate is at most 13,000 lb/hr, at most 14,000 lb/hr, at most 15,000 lb/hr, at most 16,000 lb/hr, at most 17,000 lb/hr, at most 18,000 lb/hr, at most 19,000 lb/hr, at most 20,000 lb/hr, at most 21,000 lb/hr, at most 22,000 lb/hr, at most 23,000 lb/hr, at most 24,000 lb/hr, at most 25,000 lb/hr, at most 26,000 lb/hr, at most 27,000 lb/hr, at most 28,000 lb/hr, at most 29,000 lb/hr, at most 30,000 lb/hr, at most 31,000 lb/hr, at most 32,000 lb/hr, or at most 33,000 lb/hr.

Block 326. Referring to block 326 of FIG. 3B, in some embodiments, the method further includes subjecting a feedstock to an anaerobic digestion process (e.g., block 214 of FIG. 2A, anaerobic digestion process task 114-4 of FIG. 4, etc.) in order to produce the reformer feed gas. However, the present disclosure is not limited thereto.

In some embodiments, the feedstock is MSW (e.g., stream of unsorted MSW 450 of FIG. 4, stream of sorted MSW 452 of FIG. 4, etc.). For instance, in some embodiments, the feedstock includes a food waste, a garden waste, a wood waste, a crop waste, a food manufacture byproduct, a slaughterhouse waste, a used food fat, a used edible oil, a used grease from food, a manure, a biosolid, a sewage sludge, a trim waste, or a combination thereof. For instance, in some embodiments, the crop waste includes straw (e.g., wheat straw, soybean straw), hay, corn stover, rice hulls, or a combination thereof (e.g., block 206 of FIG. 2A).

In some embodiments, the feedstock consists of between 1,000 tons and 15,000 tons of material per day, between 1,200 tons and 14,800 tons of material per day, between 1,400 tons and 14,600 tons of material per day, between 1,600 tons and 14,400 tons of material per day, between 1,800 tons and 14,200 tons of material per day, between 2,000 tons and 14,000 tons of material per day, between 2,200 tons and 13,800 tons of material per day, between 2,400 tons and 13,600 tons of material per day, between 2,600 tons and 13,400 tons of material per day, between 2,800 tons and 13,200 tons of material per day, between 3,000 tons and 13,000 tons of material per day, between 3,200 tons and 12,800 tons of material per day, between 3,400 tons and 12,600 tons of material per day, between 3,600 tons and 12,400 tons of material per day, between 3,800 tons and 12,200 tons of material per day, between 4,000 tons and 12,000 tons of material per day, between 4,200 tons and 11,800 tons of material per day, between 4,400 tons and 11,600 tons of material per day, between 4,600 tons and 11,400 tons of material per day, between 4,800 tons and 11,200 tons of material per day, between 5,000 tons and 11,000 tons of material per day, between 5,200 tons and 10,800 tons of material per day, between 5,400 tons and 10,600 tons of material per day, between 5,600 tons and 10,400 tons of material per day, between 5,800 tons and 10,200 tons of material per day, between 6,000 tons and 10,000 tons of material per day, between 6,200 tons and 9,800 tons of material per day, between 6,400 tons and 9,600 tons of material per day, between 6,600 tons and 9,400 tons of material per day, between 6,800 tons and 9,200 tons of material per day, between 7,000 tons and 9,000 tons of material per day, between 7,200 tons and 8,800 tons of material per day, between 7,400 tons and 8,600 tons of material per day, between 7,600 tons and 8,400 tons of material per day, or between 7,800 tons and 8,200 tons of material. In some embodiments, the feedstock includes less than 1,000 tons of material per day, less than 1,500 tons of material per day, less than 2,000 tons of material per day, less than 2,500 tons of material per day, less than 3,000 tons of material per day, less than 3,500 tons of material per day, less than 4,000 tons of material per day, less than 4,500 tons of material per day, less than 5,000 tons of material per day, less than 5,500 tons of material per day, less than 6,000 tons of material per day, less than 6,500 tons of material per day, less than 7,000 tons of material per day, less than 7,500 tons of material per day, less than 8,000 tons of material per day, less than 8,500 tons of material per day, less than 9,000 tons of material per day, less than 9,500 tons of material per day, less than 10,000 tons of material per day, less than 10,500 tons of material per day, less than 11,000 tons of material per day, less than 11,500 tons of material per day, less than 12,000 tons of material per day, less than 12,500 tons of material per day, less than 13,000 tons of material per day, less than 13,500 tons of material per day, less than 14,000 tons of material per day, less than 14,500 tons of material per day, less than 15,000 tons of material per day, or less than 15,500 tons of material. In some embodiments, the feedstock includes more than 1,000 tons of material per day, more than 1,500 tons of material per day, more than 2,000 tons of material per day, more than 2,500 tons of material per day, more than 3,000 tons of material per day, more than 3,500 tons of material per day, more than 4,000 tons of material per day, more than 4,500 tons of material per day, more than 5,000 tons of material per day, more than 5,500 tons of material per day, more than 6,000 tons of material per day, more than 6,500 tons of material per day, more than 7,000 tons of material per day, more than 7,500 tons of material per day, more than 8,000 tons of material per day, more than 8,500 tons of material per day, more than 9,000 tons of material per day, more than 9,500 tons of material per day, more than 10,000 tons of material per day, more than 10,500 tons of material per day, more than 11,000 tons of material per day, more than 11,500 tons of material per day, more than 12,000 tons of material per day, more than 12,500 tons of material per day, more than 13,000 tons of material per day, more than 13,500 tons of material per day, more than 14,000 tons of material per day, or more than 14,500 tons of material per day.

Block 328. Referring to block 328, in some embodiments, the synthesis gas consists of between 15 vol % and 30 vol % carbon monoxide, between 15 vol % and 29 vol % carbon monoxide, between 16 vol % and 30 vol % carbon monoxide, between 16 vol % and 29 vol % carbon monoxide, between 16 vol % and 28 vol % carbon monoxide, between 17 vol % and 29 vol % carbon monoxide, between 17 vol % and 28 vol % carbon monoxide, between 17 vol % and 27 vol % carbon monoxide, between 18 vol % and 28 vol % carbon monoxide, between 18 vol % and 27 vol % carbon monoxide, between 18 vol % and 26 vol % carbon monoxide, between 19 vol % and 27 vol % carbon monoxide, between 19 vol % and 26 vol % carbon monoxide, between 19 vol % and 25 vol % carbon monoxide, between 20 vol % and 26 vol % carbon monoxide, between 20 vol % and 25 vol % carbon monoxide, between 20 vol % and 24 vol % carbon monoxide, between 20 vol % and 23 vol % carbon monoxide, between 21 vol % and 25 vol % carbon monoxide, between 21 vol % and 24 vol % carbon monoxide, between 21 vol % and 23 vol % carbon monoxide, between 22 vol % and 24 vol % carbon monoxide, between 20 vol % and 22 vol % carbon monoxide, or between 21 vol % and 22 vol % carbon monoxide. In some embodiments, the synthesis gas consists of less than 15 vol % carbon monoxide, less than 15.5 vol % carbon monoxide, less than 16 vol % carbon monoxide, less than 16.5 vol % carbon monoxide, less than 17 vol % carbon monoxide, less than 17.5 vol % carbon monoxide, less than 18 vol % carbon monoxide, less than 18.5 vol % carbon monoxide, less than 19 vol % carbon monoxide, less than 19.5 vol % carbon monoxide, less than 20 vol % carbon monoxide, less than 20.5 vol % carbon monoxide, less than 21 vol % carbon monoxide, less than 21.5 vol % carbon monoxide, less than 22 vol % carbon monoxide, less than 22.5 vol % carbon monoxide, less than 23 vol % carbon monoxide, less than 23.5 vol % carbon monoxide, less than 24 vol % carbon monoxide, less than 24.5 vol % carbon monoxide, less than 25 vol % carbon monoxide, less than 25.5 vol % carbon monoxide, less than 26 vol % carbon monoxide, less than 26.5 vol % carbon monoxide, less than 27 vol % carbon monoxide, less than 27.5 vol % carbon monoxide, less than 28 vol % carbon monoxide, less than 28.5 vol % carbon monoxide, less than 29 vol % carbon monoxide, less than 29.5 vol % carbon monoxide, or less than 30 vol % carbon monoxide. In some embodiments, the synthesis gas consists of more than 15 vol % carbon monoxide, more than 15.5 vol % carbon monoxide, more than 16 vol % carbon monoxide, more than 16.5 vol % carbon monoxide, more than 17 vol % carbon monoxide, more than 17.5 vol % carbon monoxide, more than 18 vol % carbon monoxide, more than 18.5 vol % carbon monoxide, more than 19 vol % carbon monoxide, more than 19.5 vol % carbon monoxide, more than 20 vol % carbon monoxide, more than 20.5 vol % carbon monoxide, more than 21 vol % carbon monoxide, more than 21.5 vol % carbon monoxide, more than 22 vol % carbon monoxide, more than 22.5 vol % carbon monoxide, more than 23 vol % carbon monoxide, more than 23.5 vol % carbon monoxide, more than 24 vol % carbon monoxide, more than 24.5 vol % carbon monoxide, more than 25 vol % carbon monoxide, more than 25.5 vol % carbon monoxide, more than 26 vol % carbon monoxide, more than 26.5 vol % carbon monoxide, more than 27 vol % carbon monoxide, more than 27.5 vol % carbon monoxide, more than 28 vol % carbon monoxide, more than 28.5 vol % carbon monoxide, more than 29 vol % carbon monoxide, more than 29.5 vol % carbon monoxide, or more than 30 vol % carbon monoxide.

In some embodiments, the synthesis gas consists of between 3 vol % and 22 vol % carbon dioxide, between 4 vol % and 21 vol % carbon dioxide, between 5 vol % and 20 vol % carbon dioxide, between 6 vol % and 19 vol % carbon dioxide, between 7 vol % and 18 vol % carbon dioxide, between 8 vol % and 17 vol % carbon dioxide, between 9 vol % and 16 vol % carbon dioxide, between 10 vol % and 15 vol % carbon dioxide, between 11 vol % and 14 vol % carbon dioxide, or between 12 vol % and 13 vol % carbon. In some embodiments, the synthesis gas consists of less than 4 vol % carbon dioxide, less than 5 vol % carbon dioxide, less than 6 vol % carbon dioxide, less than 7 vol % carbon dioxide, less than 8 vol % carbon dioxide, less than 9 vol % carbon dioxide, less than 10 vol % carbon dioxide, less than 11 vol % carbon dioxide, less than 12 vol % carbon dioxide, less than 13 vol % carbon dioxide, less than 14 vol % carbon dioxide, less than 15 vol % carbon dioxide, less than 16 vol % carbon dioxide, less than 17 vol % carbon dioxide, less than 18 vol % carbon dioxide, less than 19 vol % carbon dioxide, or less than 20 vol % carbon dioxide. In some embodiments, the synthesis gas consists of more than 3 vol % carbon dioxide, more than 4 vol % carbon dioxide, more than 5 vol % carbon dioxide, more than 6 vol % carbon dioxide, more than 7 vol % carbon dioxide, more than 8 vol % carbon dioxide, more than 9 vol % carbon dioxide, more than 10 vol % carbon dioxide, more than 11 vol % carbon dioxide, more than 12 vol % carbon dioxide, more than 13 vol % carbon dioxide, more than 14 vol % carbon dioxide, more than 15 vol % carbon dioxide, more than 16 vol % carbon dioxide, more than 17 vol % carbon dioxide, more than 18 vol % carbon dioxide, more than 19 vol % carbon dioxide, or more than 20 vol % carbon dioxide.

In some embodiments, for the synthesis gas, the first portion of hydrogen is between 60 vol % and 78 vol %, the second portion of carbon monoxide is between 14 vol % and 33 vol %, and the third portion of carbon dioxide is between 5 vol % and 20 vol %. Furthermore, in some embodiments, for the synthesis gas, the first portion of hydrogen is between 66 vol % and 78 vol %, the second portion of carbon monoxide is between 14 vol % and 33 vol %, and the third portion of carbon dioxide is between 5 vol % and 20 vol %. In some embodiments, for the synthesis gas, the first portion of hydrogen is between 60 vol % and 78 vol %, the second portion of carbon monoxide is between 14 vol % and 33 vol %, and the third portion of carbon dioxide is between 5 vol % and 20 vol %. Furthermore, in some embodiments, for the synthesis gas, the first portion of hydrogen is between 66 vol % and 78 vol % and the second portion of carbon monoxide is between 20 vol % and 25 vol %.

For instance, in some embodiments, the first portion of hydrogen for the synthesis gas is between 60 vol % and 78 vol %, between 60 vol % and 77 vol %, between 60 vol % and 76 vol %, between 61 vol % and 76 vol %, between 61 vol % and 74 vol %, between 61 vol % and 70 vol %, between 63 vol % and 77 vol %, between 63 vol % and 75 vol %, between 63 vol % and 70 vol %, between 65 vol % and 77 vol %, between 65 vol % and 75 vol %, between 65 vol % and 73 vol %, between 67 vol % and 77 vol %, between 67 vol % and 76 vol %, between 67 vol % and 75 vol %, between 68 vol % and 76 vol %, between 68 vol % and 75 vol %, between 68 vol % and 75 vol %, between 69 vol % and 75 vol %, between 69 vol % and 74 vol %, between 69 vol % and 73 vol %, between 70 vol % and 74 vol %, between 70 vol % and 73 vol %, between 70 vol % and 72 vol %, between 71 vol % and 72 vol %, or between 71 vol % and 75 vol %. Moreover, in some embodiments, the first portion of hydrogen for the synthesis gas is less than 60 vol %, less than 61 vol %, less than 62 vol %, less than 63 vol %, less than 64 vol %, less than 65 vol %, less than 66 vol %, less than 67 vol %, less than 68 vol %, less than 69 vol %, less than 70 vol %, less than 71 vol %, less than 72 vol %, less than 73 vol %, less than 74 vol %, less than 75 vol %, less than 76 vol %, or less than 77 vol %. In some embodiments, the first portion of hydrogen for the synthesis gas is more than 60 vol %, more than 61 vol %, more than 62 vol %, more than 63 vol %, more than 64 vol %, more than 65 vol %, more than 66 vol %, more than 67 vol %, more than 68 vol %, more than 69 vol %, more than 70 vol %, more than 71 vol %, more than 72 vol %, more than 73 vol %, more than 74 vol %, more than 75 vol %, more than 76 vol %, or more than 77 vol %.

In some embodiments, the second portion of carbon monoxide for the synthesis gas is between 14 vol % and 33 vol %, between 14 vol % and 32 vol %, between 14 vol % and 31 vol %, between 15 vol % and 33 vol %, between 15 vol % and 32 vol %, between 15 vol % and 31 vol %, between 15 vol % and 30 vol %, between 16 vol % and 32 vol %, between 16 vol % and 31 vol %, between 16 vol % and 30 vol %, between 17 vol % and 31 vol %, between 17 vol % and 30 vol %, between 17 vol % and 29 vol %, between 18 vol % and 30 vol %, between 18 vol % and 29 vol %, between 18 vol % and 28 vol %, between 19 vol % and 29 vol %, between 19 vol % and 28 vol %, between 19 vol % and 17 vol %, between 20 vol % and 28 vol %, between 20 vol % and 27 vol %, between 20 vol % and 26 vol %, between 21 vol % and 27 vol %, between 21 vol % and 26 vol %, between 21 vol % and 25 vol %, between 22 vol % and 25 vol %, between 22 vol % and 24 vol %, between 22 vol % and 23 vol %, between 23 vol % and 30 vol %, or between 23 vol % and 27 vol %. Furthermore, in some embodiments, the second portion of carbon monoxide for the synthesis gas is more than 14 vol %, more than 15 vol %, more than 16 vol %, more than 17 vol %, more than 18 vol %, more than 19 vol % more than 20 vol %, more than 21 vol %, more than 22 vol %, more than 23 vol %, more than 24 vol %, more than 25 vol %, more than 26 vol %, more than 27 vol %, more than 28 vol %, more than 29 vol %, more than 30 vol %, more than 31 vol %, more than 32 vol %, or more than 33 vol %. In some embodiments, the second portion of carbon monoxide for the synthesis gas is less than 14 vol %, less than 15 vol %, less than 16 vol %, less than 17 vol %, less than 18 vol %, less than 19 vol % less than 20 vol %, less than 21 vol %, less than 22 vol %, less than 23 vol %, less than 24 vol %, less than 25 vol %, less than 26 vol %, less than 27 vol %, less than 28 vol %, less than 29 vol %, less than 30 vol %, less than 31 vol %, less than 32 vol %, or less than 33 vol %.

Furthermore, in some embodiments, the third portion of carbon dioxide for the synthesis is between 5 vol % and 20 vol %, between 5 vol % and 19 vol %, between 5 vol % and 18 vol %, between 6 vol % and 20 vol %, between 6 vol %, and 19 vol %, between 6 vol % and 18 vol %, between 7 vol % and 19 vol %, between 7 vol % and 18 vol %, between 7 vol % and 17 vol %, between 8 vol % and 18 vol %, between 8 vol % and 17 vol %, between 8 vol % and 16 vol %, between 9 vol % and 17 vol %, between 9 vol % and 16 vol %, between 9 vol % and 15 vol %, between 10 vol % and 16 vol %, between 10 vol % and 15 vol %, between 10 vol % and 14 vol %, between 11 vol % and 15 vol %, between 11 vol % and 14 vol %, between 11 vol % and 13 vol %, between 12 vol % and 15 vol %, between 12 vol % and 14, between 12 vol % and 13 vol %, or between 13 vol % and 17 vol %. In some embodiments, the third portion of carbon dioxide for the synthesis is less than 5 vol %, less than 6 vol %, less than 7 vol %, less than 8 vol %, less than 9 vol %, less than 10 vol %, less than 11 vol %, less than 12 vol %, less than 13 vol %, less than 14 vol %, less than 15 vol %, less than 16 vol %, less than 17 vol %, less than 18 vol %, less than 19 vol %, or less than 20 vol %. In some embodiments, the third portion of carbon dioxide for the synthesis is more than 5 vol %, more than 6 vol %, more than 7 vol %, more than 8 vol %, more than 9 vol %, more than 10 vol %, more than 11 vol %, more than 12 vol %, more than 13 vol %, more than 14 vol %, more than 15 vol %, more than 16 vol %, more than 17 vol %, more than 18 vol %, more than 19 vol %, or more than 20 vol %.

Accordingly, in some embodiments, the first portion of hydrogen for the synthesis gas is less than 60 vol %, less than 61 vol %, less than 62 vol %, less than 63 vol %, less than 64 vol %, less than 65 vol %, less than 66 vol %, less than 67 vol %, less than 68 vol %, less than 69 vol %, less than 70 vol %, less than 71 vol %, less than 72 vol %, less than 73 vol %, less than 74 vol %, less than 75 vol %, less than 76 vol %, or less than 77 vol %; the second portion of carbon monoxide for the synthesis gas is less than 14 vol %, less than 15 vol %, less than 16 vol %, less than 17 vol %, less than 18 vol %, less than 19 vol % less than 20 vol %, less than 21 vol %, less than 22 vol %, less than 23 vol %, less than 24 vol %, less than 25 vol %, less than 26 vol %, less than 27 vol %, less than 28 vol %, less than 29 vol %, less than 30 vol %, less than 31 vol %, less than 32 vol %, or less than 33 vol %; and the third portion of carbon dioxide for the synthesis is less than 5 vol %, less than 6 vol %, less than 7 vol %, less than 8 vol %, less than 9 vol %, less than 10 vol %, less than 11 vol %, less than 12 vol %, less than 13 vol %, less than 14 vol %, less than 15 vol %, less than 16 vol %, less than 17 vol %, less than 18 vol %, less than 19 vol %, or less than 20 vol %.

Furthermore, in some embodiments, the first portion of hydrogen for the synthesis gas is more than 60 vol %, more than 61 vol %, more than 62 vol %, more than 63 vol %, more than 64 vol %, more than 65 vol %, more than 66 vol %, more than 67 vol %, more than 68 vol %, more than 69 vol %, more than 70 vol %, more than 71 vol %, more than 72 vol %, more than 73 vol %, more than 74 vol %, more than 75 vol %, more than 76 vol %, or more than 77 vol %; the second portion of carbon monoxide for the synthesis gas is more than 14 vol %, more than 15 vol %, more than 16 vol %, more than 17 vol %, more than 18 vol %, more than 19 vol % more than 20 vol %, more than 21 vol %, more than 22 vol %, more than 23 vol %, more than 24 vol %, more than 25 vol %, more than 26 vol %, more than 27 vol %, more than 28 vol %, more than 29 vol %, more than 30 vol %, more than 31 vol %, more than 32 vol %, or more than 33 vol %; and the third portion of carbon dioxide for the synthesis is more than 5 vol %, more than 6 vol %, more than 7 vol %, more than 8 vol %, more than 9 vol %, more than 10 vol %, more than 11 vol %, more than 12 vol %, more than 13 vol %, more than 14 vol %, more than 15 vol %, more than 16 vol %, more than 17 vol %, more than 18 vol %, more than 19 vol %, or more than 20 vol %.

Accordingly, the synthesis gas includes the first portion of hydrogen, the second portion of carbon monoxide, and the third portion of cardon dioxide in a non-negligible amounts, which allows for the first portion of hydrogen, the second portion of carbon monoxide, and the third portion of cardon dioxide of the synthesis gas to form a part of a reaction the synthesis gas is utilized with (e.g., block 234 of FIG. 2B, block 340 of FIG. 3C, etc.)

Block 330. Referring to block 330, in some embodiments, the first portion of hydrogen is augmented by an auxiliary hydrogen supply.

Block 332. Referring to block 332, in some embodiments, the methane reformer includes a steam methane reformer and/or an auto-thermal reformer. For instance, in some embodiments, the methane reformer and/or the auto-thermal reformer are capable of reforming both carbon dioxide and methane in the same reactor. However, the present disclosure is not limited thereto. For instance, in some such embodiments, the auto-thermal reformer is utilized for producing 100 or more tons of product (e.g., 1,000 tons of methanol product) a day.

Block 334. Referring to block 334, the method 300 further includes subjecting the synthesis gas to a methanol synthesis reactor (e.g., methanol synthesis reactor task 114-9 of FIG. 5, methanol synthesis reactor task 114-2 of FIG. 5).

Block 336. Referring to block 336, in some embodiments, the methanol synthesis reactor is a steam-raising reactor. For instance, in some embodiments, the methanol synthesis reactor is an axial flow steam-raising reactor or radial flow steam-raising reactor. In some embodiment, the methanol synthesis reactor includes a catalyst on a surface of the steam-raising reactor, such as tube surface and/or a shell surface (e.g., an interior surface and/or an exterior surface) of the steam raising reactor. However, the present disclosure is not limited thereto.

Block 338. Referring to block 338, in some embodiments, the subjecting the synthesis gas to the methanol synthesis reactor is conducted at a pressure of from about 800 pound-force per square inch (PSI) to about 3,200 PSI, from about 1,000 PSI to about 3,000 PSI, from about 1,200 PSI to about 2,800 PSI, from about 1,400 PSI to about 2,600 PSI, from about 1,600 PSI to about 2,400 PSI, or from about 1,800 PSI to about 2,200 PSI. In some embodiments, the subjecting the synthesis gas to the methanol synthesis reactor is conducted at a pressure of less than 1,100 PSI, less than 1,300 PSI, less than 1,500 PSI, less than 1,700 PSI, less than 1,900 PSI, less than 2,100 PSI, less than 2,300 PSI, less than 2,500 PSI, less than 2,700 PSI, less than 2,900 PSI, or less than 3,100 PSI. In some embodiments, the subjecting the synthesis gas to the methanol synthesis reactor is conducted at a pressure between 55 bar and 220 bar, between 65 bar and 210 bar, between 75 bar and 200 bar, between 85 bar and 190 bar, between 95 bar and 180 bar, between 105 bar and 170 bar, between 115 bar and 160 bar, between 125 bar and 150 bar, or between 135 bar and 140 bar. In some embodiments, the subjecting the synthesis gas to the methanol synthesis reactor is at least 55 bar, at least 65 bar, at least 75 bar, at least 85 bar, at least 95 bar, at least 105 bar, at least 115 bar, at least 125 bar, at least 135 bar, at least 145 bar, at least 155 bar, at least 165 bar, at least 175 bar, at least 185 bar, at least 195 bar, at least 205 bar, at least 215 bar, or at least 220 bar. In some embodiments, the subjecting the synthesis gas to the methanol synthesis reactor is at most 55 bar, at most 65 bar, at most 75 bar, at most 85 bar, at most 95 bar, at most 105 bar, at most 115 bar, at most 125 bar, at most 135 bar, at most 145 bar, at most 155 bar, at most 165 bar, at most 175 bar, at most 185 bar, at most 195 bar, at most 205 bar, at most 215 bar, or at most 220 bar.

In some embodiments, a reciprocating compressor is utilized to provide the pressure of the subjecting the synthesis gas to the methanol synthesis reactor. For instance, in some embodiments, the reciprocating compressing is utilized to generate a pressure between about 100 pounds gauge pressure and 1200 pounds gauge pressure, about 200 pounds gauge pressure and 1200 pounds gauge pressure, about 200 pounds gauge pressure and 1000 pounds gauge pressure, about 200 pounds gauge pressure and 800 pounds gauge pressure, about 300 pounds gauge pressure and 800 pounds gauge pressure, or about 200 pounds gauge pressure and 500 pounds gauge pressure. In some embodiments, the reciprocating compressing is utilized to generate a pressure of at least 100 pounds gauge pressure, at least 200 pounds gauge pressure, at least 400 pounds gauge pressure, at least 600 pounds gauge pressure, at least 800 pounds gauge pressure, at least 1000 pounds gauge pressure, or at least 1,200 pounds gauge pressure. In some embodiments, the reciprocating compressing is utilized to generate a pressure of at most 200 pounds gauge pressure, at most 400 pounds gauge pressure, at most 600 pounds gauge pressure, at most 800 pounds gauge pressure, at most 1000 pounds gauge pressure, or at most 1,200 pounds gauge pressure.

Block 340. Referring to block 340, the method 300 includes using the methanol synthesis reactor (e.g., methanol synthesis reactor task 114-9 of FIG. 4, methanol synthesis reactor task 114-3 of FIG. 5, etc.) to produce a methanol product and a purge gas stream from the synthesis gas. Accordingly, the methanol reactor is be configured to take at least a portion of the synthesis gas from the methane reformer (e.g., block 304 of FIG. 3A) as an input and produce a methanol product (e.g., methanol product 482 of FIG. 4, methanol product 588 of FIG. 5, etc.) and/or a purge gas stream (e.g., auxiliary hydrogen supply 492 of FIG. 4).

Block 342. Referring to block 342, in some embodiments, the synthesis gas during the subjecting the synthesis gas to the methanol reformer or using the methanol synthesis reactor to product the methanol product has a second mass flow rate of between 5,000 kg/h and 20,000 kg/h, between 5,250 kg/h and 19,750 kg/h, between 5,500 kg/h and 19,500 kg/h, between 5,750 kg/h and 19,250 kg/h, between 6,000 kg/h and 19,000 kg/h, between 6,250 kg/h and 18,750 kg/h, between 6,500 kg/h and 18,500 kg/h, between 6,750 kg/h and 18,250 kg/h, between 7,000 kg/h and 18,000 kg/h, between 7,250 kg/h and 17,750 kg/h, between 7,500 kg/h and 17,500 kg/h, between 7,750 kg/h and 17,250 kg/h, between 8,000 kg/h and 17,000 kg/h, between 8,250 kg/h and 16,750 kg/h, between 8,500 kg/h and 16,500 kg/h, between 8,750 kg/h and 16,250 kg/h, between 9,000 kg/h and 16,000 kg/h, between 9,250 kg/h and 15,750 kg/h, between 9,500 kg/h and 15,500 kg/h, between 9,750 kg/h and 15,250 kg/h, between 10,000 kg/h and 15,000 kg/h, between 10,250 kg/h and 14,750 kg/h, between 10,500 kg/h and 14,500 kg/h, between 10,750 kg/h and 14,250 kg/h, between 11,000 kg/h and 14,000 kg/h, between 11,250 kg/h and 13,750 kg/h, between 11,500 kg/h and 13,500 kg/h, between 11,750 kg/h and 13,250 kg/h, between 12,000 kg/h and 13,000 kg/h, or between 12,250 kg/h and 12,750 kg/h. In some embodiments, the mass flow rate of the reformer feed gas is at least 6,000 kg/h, at least 6,500 kg/h, at least 7,000 kg/h, at least 7,500 kg/h, at least 8,000 kg/h, at least 8,500 kg/h, at least 9,000 kg/h, at least 9,500 kg/h, at least 10,000 kg/h, at least 10,500 kg/h, at least 11,000 kg/h, at least 11,500 kg/h, at least 12,000 kg/h, at least 12,500 kg/h, at least 13,000 kg/h, at least 13,500 kg/h, at least 14,000 kg/h, at least 14,500 kg/h, at least 15,000 kg/h, at least 15,500 kg/h, at least 16,000 kg/h, at least 16,500 kg/h, at least 17,000 kg/h, at least 17,500 kg/h, at least 18,000 kg/h, at least 18,500 kg/h, at least 19,000 kg/h, at least 19,500 kg/h, or at least 20,000 kg/h. In some embodiments, the mass flow rate of the reformer feed gas is at most 6,000 kg/h, at most 6,500 kg/h, at most 7,000 kg/h, at most 7,500 kg/h, at most 8,000 kg/h, at most 8,500 kg/h, at most 9,000 kg/h, at most 9,500 kg/h, at most 10,000 kg/h, at most 10,500 kg/h, at most 11,000 kg/h, at most 11,500 kg/h, at most 12,000 kg/h, at most 12,500 kg/h, at most 13,000 kg/h, at most 13,500 kg/h, at most 14,000 kg/h, at most 14,500 kg/h, at most 15,000 kg/h, at most 15,500 kg/h, at most 16,000 kg/h, at most 16,500 kg/h, at most 17,000 kg/h, at most 17,500 kg/h, at most 18,000 kg/h, at most 18,500 kg/h, at most 19,000 kg/h, at most 19,500 kg/h, or at most 20,000 kg/h.

In some embodiments, the first mass flow rate is between 11,000 lb/hr and 44,000 lb/hr, between 14,000 lb/hr and 42,000 lb/hr, between 15,000 lb/hr and 41,000 lb/hr, between 16,000 lb/hr and 40,000 lb/hr, between 14,000 lb/hr and 32,000 lb/hr, between 15,000 lb/hr and 31,000 lb/hr, between 16,000 lb/hr and 30,000 lb/hr, between 19,000 lb/hr and 29,000 lb/hr, between 20,000 lb/hr and 28,000 lb/hr, between 21,000 lb/hr and 27,000 lb/hr, between 22,000 lb/hr and 26,000 lb/hr, or between 23,000 lb/hr and 25,000 lb/hr. In some embodiments, the first mass flow rate is at least 11,000 lb/hr, at least 13,000 lb/hr, at least 14,000 lb/hr, at least 15,000 lb/hr, at least 16,000 lb/hr, at least 17,000 lb/hr, at least 18,000 lb/hr, at least 19,000 lb/hr, at least 20,000 lb/hr, at least 21,000 lb/hr, at least 22,000 lb/hr, at least 23,000 lb/hr, at least 24,000 lb/hr, at least 25,000 lb/hr, at least 26,000 lb/hr, at least 27,000 lb/hr, at least 28,000 lb/hr, at least 29,000 lb/hr, at least 30,000 lb/hr, at least 31,000 lb/hr, at least 32,000 lb/hr, at least 33,000 lb/hr, at least 34,000 lb/hr, at least 35,000 lb/hr, at least 36,000 lb/hr, at least 37,000 lb/hr, at least 38,000 lb/hr, at least 39,000 lb/hr, at least 40,000 lb/hr, at least 41,000 lb/hr, at least 42,000 lb/hr, at least 43,000 lb/hr, or at least 44,000 lb/hr. In some embodiments, the first mass flow rate is at most 13,000 lb/hr, at most 14,000 lb/hr, at most 15,000 lb/hr, at most 16,000 lb/hr, at most 17,000 lb/hr, at most 18,000 lb/hr, at most 19,000 lb/hr, at most 20,000 lb/hr, at most 21,000 lb/hr, at most 22,000 lb/hr, at most 23,000 lb/hr, at most 24,000 lb/hr, at most 25,000 lb/hr, at most 26,000 lb/hr, at most 27,000 lb/hr, at most 28,000 lb/hr, at most 29,000 lb/hr, at most 30,000 lb/hr, at most 31,000 lb/hr, at most 32,000 lb/hr, at most 33,000 lb/hr, at most 34,000 lb/hr, at most 35,000 lb/hr, at most 36,000 lb/hr, at most 37,000 lb/hr, at most 38,000 lb/hr, at most 39,000 lb/hr, at most 40,000 lb/hr, at most 41,000 lb/hr, at most 42,000 lb/hr, at most 43,000 lb/hr, or at most 44,000 lb/hr.

Block 344. Referring to block 344, in some embodiments, the using the methanol synthesis reactor to produce the methanol product provides between 20 tons and 250 tons of methanol product a day, between 25 tons and 250 tons of methanol product a day, between 25 tons and 245 tons of methanol product a day, between 30 tons and 250 tons of methanol product a day, between 30 tons and 245 tons of methanol product a day, between 30 tons and 240 tons of methanol product a day, between 35 tons and 235 tons of methanol product a day, between 40 tons and 250 tons of methanol product a day, between 40 tons and 240 tons of methanol product a day, between 40 tons and 230 tons of methanol product a day, between 45 tons and 225 tons of methanol product a day, between 50 tons and 220 tons of methanol product a day, between 50 tons and 250 tons of methanol product a day, between 50 tons and 230 tons of methanol product a day, between 55 tons and 215 tons of methanol product a day, between 60 tons and 250 tons of methanol product a day, between 60 tons and 230 tons of methanol product a day, between 60 tons and 210 tons of methanol product a day, between 65 tons and 205 tons of methanol product a day, between 70 tons and 250 tons of methanol product a day, between 70 tons and 230 tons of methanol product a day, between 70 tons and 210 tons of methanol product a day, between 70 tons and 200 tons of methanol product a day, between 75 tons and 195 tons of methanol product a day, between 80 tons and 190 tons of methanol product a day, between 85 tons and 185 tons of methanol product a day, between 90 tons and 180 tons of methanol product a day, between 100 tons and 250 tons of methanol product a day, between 125 tons and 250 tons of methanol product a day, between 100 tons and 200 tons of methanol product a day, between 75 tons and 250 tons of methanol product a day, between 75 tons and 200 tons of methanol product a day, between 75 tons and 150 tons of methanol product a day, between 70 tons and 125 tons of methanol product a day, between 65 tons and 115 tons of methanol product a day, between 65 tons and 110 tons of methanol product a day, between 70 tons and 115 tons of methanol product a day, between 70 tons and 110 tons of methanol product a day, between 70 and 105 tons of methanol product a day, between 70 tons and 100 tons of methanol product a day, between 75 tons and 115 tons of methanol product a day, between 75 tons and 110 tons of methanol product a day, between 75 tons and 105 tons of methanol product a day, between 75 and 100 tons of methanol product a day, between 80 tons and 110 tons of methanol product a day, between 80 tons and 105 tons of methanol product a day, between 80 and 100 tons of methanol product a day, between 85 tons and 115 tons of methanol product a day, between 85 tons and 110 tons of methanol product a day, between 85 and 105 tons of methanol product a day, between 90 tons and 110 tons of methanol product a day, between 90 tons and 105 tons of methanol product a day, between 90 and 100 tons of methanol product a day, between 90 tons and 95 tons of methanol product a day, or between 80 and 90 tons of methanol product a day. In some embodiments, the using the methanol synthesis reactor to produce the methanol product provides at most 25 tons of methanol product a day, at most 26 tons of methanol product a day, at most 27 tons of methanol product a day, at most 28 tons of methanol product a day, at most 29 tons of methanol product a day, at most 30 tons of methanol product a day, at most 31 tons of methanol product a day, at most 32 tons of methanol product a day, at most 33 tons of methanol product a day, at most 34 tons of methanol product a day, at most 35 tons of methanol product a day, at most 36 tons of methanol product a day, at most 37 tons of methanol product a day, at most 38 tons of methanol product a day, at most 39 tons of methanol product a day, at most 40 tons of methanol product a day, at most 41 tons of methanol product a day, at most 42 tons of methanol product a day, at most 43 tons of methanol product a day, at most 44 tons of methanol product a day, at most 45 tons of methanol product a day, at most 46 tons of methanol product a day, at most 47 tons of methanol product a day, at most 48 tons of methanol product a day, at most 49 tons of methanol product a day, at most 50 tons of methanol product a day, at most 51 tons of methanol product a day, at most 52 tons of methanol product a day, at most 53 tons of methanol product a day, at most 54 tons of methanol product a day, at most 55 tons of methanol product a day, at most 56 tons of methanol product a day, at most 57 tons of methanol product a day, at most 58 tons of methanol product a day, at most 59 tons of methanol product a day, at most 60 tons of methanol product a day, at most 61 tons of methanol product a day, at most 62 tons of methanol product a day, at most 63 tons of methanol product a day, at most 64 tons of methanol product a day, at most 65 tons of methanol product a day, at most 66 tons of methanol product a day, at most 67 tons of methanol product a day, at most 68 tons of methanol product a day, at most 69 tons of methanol product a day, at most 70 tons of methanol product a day, at most 71 tons of methanol product a day, at most 72 tons of methanol product a day, at most 73 tons of methanol product a day, at most 74 tons of methanol product a day, at most 75 tons of methanol product a day, at most 76 tons of methanol product a day, at most 77 tons of methanol product a day, at most 78 tons of methanol product a day, at most 79 tons of methanol product a day, at most 80 tons of methanol product a day, at most 81 tons of methanol product a day, at most 82 tons of methanol product a day, at most 83 tons of methanol product a day, at most 84 tons of methanol product a day, at most 85 tons of methanol product a day, at most 86 tons of methanol product a day, at most 87 tons of methanol product a day, at most 88 tons of methanol product a day, at most 89 tons of methanol product a day, at most 90 tons of methanol product a day, at most 91 tons of methanol product a day, at most 92 tons of methanol product a day, at most 93 tons of methanol product a day, at most 94 tons of methanol product a day, at most 95 tons of methanol product a day, at most 96 tons of methanol product a day, at most 97 tons of methanol product a day, at most 98 tons of methanol product a day, at most 100 tons of methanol product a day, at most 101 tons of methanol product a day, at most 102 tons of methanol product a day, at most 103 tons of methanol product a day, at most 104 tons of methanol product a day, at most 105 tons of methanol product a day, at most 106 tons of methanol product a day, at most 107 tons of methanol product a day, at most 108 tons of methanol product a day, at most 109 tons of methanol product a day, at most 110 tons of methanol product a day, at most 115 tons of methanol product a day, at most 120 tons of methanol product a day, at most 125 tons of methanol product a day, at most 130 tons of methanol product a day, at most 135 tons of methanol product a day, at most 140 tons of methanol product a day, at most 145 tons of methanol product a day, at most 150 tons of methanol product a day, at most 155 tons of methanol product a day, at most 160 tons of methanol product a day, at most 165 tons of methanol product a day, at most 170 tons of methanol product a day, at most 175 tons of methanol product a day, at most 180 tons of methanol product a day, at most 185 tons of methanol product a day, at most 190 tons of methanol product a day, at most 195 tons of methanol product a day, at most 200 tons of methanol product a day, at most 205 tons of methanol product a day, at most 210 tons of methanol product a day, at most 215 tons of methanol product a day, at most 220 tons of methanol product a day, at most 225 tons of methanol product a day, at most 230 tons of methanol product a day, at most 235 tons of methanol product a day, at most 240 tons of methanol product a day, at most 245 tons of methanol product a day, at most 250 tons of methanol product a day, at most 255 tons of methanol product a day, at most 260 tons of methanol product a day, at most 265 tons of methanol product a day. In some embodiments, the using the methanol synthesis reactor to produce the methanol product provides at least 25 tons of methanol product a day, at least 26 tons of methanol product a day, at least 27 tons of methanol product a day, at least 28 tons of methanol product a day, at least 29 tons of methanol product a day, at least 30 tons of methanol product a day, at least 31 tons of methanol product a day, at least 32 tons of methanol product a day, at least 33 tons of methanol product a day, at least 34 tons of methanol product a day, at least 35 tons of methanol product a day, at least 36 tons of methanol product a day, at least 37 tons of methanol product a day, at least 38 tons of methanol product a day, at least 39 tons of methanol product a day, at least 40 tons of methanol product a day, at least 41 tons of methanol product a day, at least 42 tons of methanol product a day, at least 43 tons of methanol product a day, at least 44 tons of methanol product a day, at least 45 tons of methanol product a day, at least 46 tons of methanol product a day, at least 47 tons of methanol product a day, at least 48 tons of methanol product a day, at least 49 tons of methanol product a day, at least 50 tons of methanol product a day, at least 51 tons of methanol product a day, at least 52 tons of methanol product a day, at least 53 tons of methanol product a day, at least 54 tons of methanol product a day, at least 55 tons of methanol product a day, at least 56 tons of methanol product a day, at least 57 tons of methanol product a day, at least 58 tons of methanol product a day, at least 59 tons of methanol product a day, at least 60 tons of methanol product a day, at least 61 tons of methanol product a day, at least 62 tons of methanol product a day, at least 63 tons of methanol product a day, at least 64 tons of methanol product a day, at least 65 tons of methanol product a day, at least 66 tons of methanol product a day, at least 67 tons of methanol product a day, at least 68 tons of methanol product a day, at least 69 tons of methanol product a day, at least 70 tons of methanol product a day, at least 71 tons of methanol product a day, at least 72 tons of methanol product a day, at least 73 tons of methanol product a day, at least 74 tons of methanol product a day, at least 75 tons of methanol product a day, at least 76 tons of methanol product a day, at least 77 tons of methanol product a day, at least 78 tons of methanol product a day, at least 79 tons of methanol product a day, at least 80 tons of methanol product a day, at least 81 tons of methanol product a day, at least 82 tons of methanol product a day, at least 83 tons of methanol product a day, at least 84 tons of methanol product a day, at least 85 tons of methanol product a day, at least 86 tons of methanol product a day, at least 87 tons of methanol product a day, at least 88 tons of methanol product a day, at least 89 tons of methanol product a day, at least 90 tons of methanol product a day, at least 91 tons of methanol product a day, at least 92 tons of methanol product a day, at least 93 tons of methanol product a day, at least 94 tons of methanol product a day, at least 95 tons of methanol product a day, at least 96 tons of methanol product a day, at least 97 tons of methanol product a day, at least 98 tons of methanol product a day, at least 100 tons of methanol product a day, at least 101 tons of methanol product a day, at least 102 tons of methanol product a day, at least 103 tons of methanol product a day, at least 104 tons of methanol product a day, at least 105 tons of methanol product a day, at least 106 tons of methanol product a day, at least 107 tons of methanol product a day, at least 108 tons of methanol product a day, at least 109 tons of methanol product a day, at least 110 tons of methanol product a day, at least 115 tons of methanol product a day, at least 120 tons of methanol product a day, at least 125 tons of methanol product a day, at least 130 tons of methanol product a day, at least 135 tons of methanol product a day, at least 140 tons of methanol product a day, at least 145 tons of methanol product a day, at least 150 tons of methanol product a day, at least 155 tons of methanol product a day, at least 160 tons of methanol product a day, at least 165 tons of methanol product a day, at least 170 tons of methanol product a day, at least 175 tons of methanol product a day, at least 180 tons of methanol product a day, at least 185 tons of methanol product a day, at least 190 tons of methanol product a day, at least 195 tons of methanol product a day, at least 200 tons of methanol product a day, at least 205 tons of methanol product a day, at least 210 tons of methanol product a day, at least 215 tons of methanol product a day, at least 220 tons of methanol product a day, at least 225 tons of methanol product a day, at least 230 tons of methanol product a day, at least 235 tons of methanol product a day, at least 240 tons of methanol product a day, at least 245 tons of methanol product a day, at least 250 tons of methanol product a day, at least 255 tons of methanol product a day, at least 260 tons of methanol product a day, at least 265 tons of methanol product a day.

Block 346. Referring to block 346, in some embodiments, a carbon efficiency of the producing the methanol product from the reformer feed gas is between 92% and 99%, between 93% and 99%, between 94% and 99%, between 95% and 99%, between 96% and 99%, between 97% and 99%, between 98% and 99%, between 95% and 98%, between 96% and 98%, between 97% and 98%, between 95% and 97%, between 96% and 97%, or between 94% and 95%. In some embodiments, the carbon efficiency of the producing the methanol product from the reformer feed gas is at least 94%, at least 94.5%, at least 95%, at least 95.5%, at least 96%, at least 96.5%, at least 97%, at least 97.5%, at least 98%, at least 98.5%, at least 99%, or at least 99.5%. In some embodiments, the carbon efficiency of the producing the methanol product from the reformer feed gas is at most 94%, at most 94.5%, at most 95%, at most 95.5%, at most 96%, at most 96.5%, at most 97%, at most 97.5%, at most 98%, at most 98.5%, at most 99%, or at most 99.5%.

Block 348. Referring to block 358, in some embodiments, the subjecting the synthesis gas to the methanol synthesis reactor and/or using the methanol synthesis reactor to produce the methanol product includes passing a subset of the synthesis gas through the methanol synthesis reactor for two or more passes. For instance, in some embodiments, the methanol synthesis reactor is configured to take at least a portion of the synthesis gas from the methane reformer as an input, which includes one or more recycle gases that enter methanol synthesis reactor with the portion of the synthesis gas and produce the methanol product and/or the purge gas stream. However, the present disclosure is not limited thereto.

In some embodiments, the passing the subset of the synthesis gas through the methanol synthesis reactor has a conversion efficiency between about 25% and about 35% each pass, between about 25% and about 34% each pass, between about 25% and about 33% each pass, between about 26% and about 35% each pass, between about 26% and about 34% each pass, between about 26% and about 33% each pass, between about 26% and about 32% each pass, between about 27% and about 33%, each pass between about 27% and about 32% each pass, between about 27% and about 31% each pass, between about 28% and about 34% each pass, between about 28% and about 33% each pass, between about 28% and about 32% each pass, between about 28% and about 31% each pass, between about 28% and about 30% each pass, between about 29% and about 33% each pass, between about 29% and about 32% each pass, between about 29% and about 31% each pass, between about 30% and about 35% each pass, or between 30% and about 33% each pass. In some embodiments, the conversion efficiency is at least 25% each pass, at least 26% each pass, at least 27% each pass, at least 28% each pass, at least 29% each pass, at least 30% each pass, at least 31% each pass, at least 32% each pass, at least 33% each pass, at least 34% each pass, or at least 35% each pass. In some embodiments, the conversion efficiency is at most 25% each pass, at most 26% each pass, at most 27% each pass, at most 28% each pass, at most 29% each pass, at most 30% each pass, at most 31% each pass, at most 32% each pass, at most 33% each pass, at most 34% each pass, or at most 35% each pass.

In some embodiments, the temperature of the synthesis gas in the methanol synthesis reactor is between 375° F. and 590° F., between 380° F. and 590° F., between 400° F. and 570° F., between 420° F. and 550° F., between 440° F. and 530° F., between 460° F. and 510° F., or between 490° F. and 490° F. In some embodiments, the temperature of the synthesis gas in the methanol synthesis reactor is at least 375° F., at least 400° F., at least 425° F., at least 450° F., at least 475° F., at least 500° F., at least 525° F., at least 550° F., at least 575° F., or at least 590° F. In some embodiments, the temperature of the synthesis gas in the methanol synthesis reactor is at most 375° F., at most 400° F., at most 425° F., at most 450° F., at most 475° F., at most 500° F., at most 525° F., at most 550° F., at most 575° F., or at most 590° F. In some embodiments, the temperature of the synthesis gas in the methanol synthesis reactor is from about 190° C. to about 310° C., from about 190° C. to about 305° C., from about 195° C. to about 305° C., from about 195° C. to about 300° C., from about 200° C. to about 305° C., from about 200° C. to about 300° C., from about 200° C. to about 295° C., from about 205° C. to about 295° C., from about 205° C. to about 290° C., from about 205° C. to about 285° C., from about 210° C. to about 285° C., from about 210° C. to about 280° C., from about 210° C. to about 275° C., from about 215° C. to about 280° C., from about 215° C. to about 275° C., from about 215° C. to about 270° C., from about 210° C. to about 285° C., from about 210° C. to about 280° C., from about 210° C. to about 275° C., from about 215° C. to about 280° C., from about 215° C. to about 275° C., from about 215° C. to about 270° C., from about 220° C. to about 275° C., from about 220° C. to about 270° C., from about 220° C. to about 265° C., from about 225° C. to about 270° C., from about 225° C. to about 265° C., from about 225° C. to about 260° C., from about 230° C. to about 265° C., from about 230° C. to about 260° C., from about 230° C. to about 255° C., from about 235° C. to about 260° C., from about 235° C. to about 255° C., from about 235° C. to about 250° C., from about 240° C. to about 255° C., from about 240° C. to about 250° C., from about 240° C. to about 245° C., from about 245° C. to about 250° C., or from about 245° C. to about 255° C. In some embodiments, the temperature of the synthesis gas in the methanol synthesis reactor is less than 310° C., less than 305° C., less than 300° C., less than 295° C., less than 290° C., less than 285° C., less than 280° C., less than 275° C., less than 270° C., less than 265° C., less than 260° C., less than 255° C., less than 250° C., less than 245° C., less than 240° C., less than 235° C., less than 230° C., less than 225° C., less than 220° C., less than 215° C., less than 210° C., or less than 205° C. In some embodiments, the temperature of the synthesis gas in the methanol synthesis reactor is less than 310° C., less than 305° C., less than 300° C., less than 295° C., less than 290° C., less than 285° C., less than 280° C., less than 275° C., less than 270° C., less than 265° C., less than 260° C., less than 255° C., less than 250° C., less than 245° C., less than 240° C., less than 235° C., less than 230° C., less than 225° C., less than 220° C., less than 215° C., less than 210° C., or less than 205° C.

In some embodiments, the passing the subset of the synthesis gas through the methanol synthesis reactor is configured to adjust a temperature of the methanol synthesis reactor. In some embodiments, the passing the subset of the synthesis gas through the methanol synthesis reactor is configured to adjust an impurities volume percentage of the methanol product. In some embodiments, the passing the subset of the synthesis gas through the methanol synthesis reactor is configured to adjust an impurities volume percentage of a feed stream for the methanol synthesis reactor, which provides the methanol product with less impurities. In some embodiments, each pass of the two or more passes of the passing the subset includes adjusting, by a compressor, a pressure of the fourth portion of the synthesis gas.

In some embodiments, the subset of the synthesis gas through the methanol synthesis reactor has a third mass flow rate between 20,000 kg/h and 78,000 kg/h, between 21,000 kg/h and 77,000 kg/h, between 22,000 kg/h and 76,000 kg/h, between 23,000 kg/h and 75,000 kg/h, between 24,000 kg/h and 74,000 kg/h, between 25,000 kg/h and 73,000 kg/h, between 26,000 kg/h and 72,000 kg/h, between 27,000 kg/h and 71,000 kg/h, between 28,000 kg/h and 70,000 kg/h, between 29,000 kg/h and 69,000 kg/h, between 30,000 kg/h and 68,000 kg/h, between 31,000 kg/h and 67,000 kg/h, between 32,000 kg/h and 66,000 kg/h, between 33,000 kg/h and 65,000 kg/h, between 34,000 kg/h and 64,000 kg/h, between 35,000 kg/h and 63,000 kg/h, between 36,000 kg/h and 62,000 kg/h, between 37,000 kg/h and 61,000 kg/h, between 38,000 kg/h and 60,000 kg/h, between 39,000 kg/h and 59,000 kg/h, between 40,000 kg/h and 58,000 kg/h, between 41,000 kg/h and 57,000 kg/h, between 42,000 kg/h and 56,000 kg/h, between 43,000 kg/h and 55,000 kg/h, between 44,000 kg/h and 54,000 kg/h, between 45,000 kg/h and 53,000 kg/h, between 46,000 kg/h and 52,000 kg/h, between 47,000 kg/h and 51,000 kg/h, or between 48,000 kg/h and 50,000 kg/h. In some embodiments, the third mass flow rate of the subset of the synthesis gas through the methanol synthesis reactor is at least 20,000 kg/hr, at least 20,500 kg/hr, at least 21,000 kg/hr, at least 21,500 kg/hr, at least 22,000 kg/hr, at least 22,500 kg/hr, at least 23,000 kg/hr, at least 23,500 kg/hr, at least 24,000 kg/hr, at least 24,500 kg/hr, at least 25,000 kg/hr, at least 25,500 kg/hr, at least 26,000 kg/hr, at least 26,500 kg/hr, at least 27,000 kg/hr, at least 27,500 kg/hr, at least 28,000 kg/hr, at least 28,500 kg/hr, at least 29,000 kg/hr, at least 29,500 kg/hr, at least 30,000 kg/hr, at least 30,500 kg/hr, at least 31,000 kg/hr, at least 31,500 kg/hr, at least 32,000 kg/hr, at least 32,500 kg/hr, at least 33,000 kg/hr, at least 33,500 kg/hr, at least 34,000 kg/hr, at least 34,500 kg/hr, at least 35,000 kg/hr, at least 35,500 kg/hr, at least 36,000 kg/hr, at least 36,500 kg/hr, at least 37,000 kg/hr, at least 37,500 kg/hr, at least 38,000 kg/hr, at least 38,500 kg/hr, at least 39,000 kg/hr, at least 39,500 kg/hr, at least 40,000 kg/hr, at least 40,500 kg/hr, at least 41,000 kg/hr, at least 41,500 kg/hr, at least 42,000 kg/hr, at least 42,500 kg/hr, at least 43,000 kg/hr, at least 43,500 kg/hr, at least 44,000 kg/hr, at least 44,500 kg/hr, at least 45,000 kg/hr, at least 45,500 kg/hr, at least 46,000 kg/hr, at least 46,500 kg/hr, at least 47,000 kg/hr, at least 47,500 kg/hr, at least 48,000 kg/hr, at least 48,500 kg/hr, at least 49,000 kg/hr, at least 49,500 kg/hr, at least 50,000 kg/hr, at least 50,500 kg/hr, at least 51,000 kg/hr, at least 51,500 kg/hr, at least 52,000 kg/hr, at least 52,500 kg/hr, at least 53,000 kg/hr, at least 53,500 kg/hr, at least 54,000 kg/hr, at least 54,500 kg/hr, at least 55,000 kg/hr, at least 55,500 kg/hr, at least 56,000 kg/hr, at least 56,500 kg/hr, at least 57,000 kg/hr, at least 57,500 kg/hr, at least 58,000 kg/hr, at least 58,500 kg/hr, at least 59,000 kg/hr, at least 59,500 kg/hr, at least 60,000 kg/hr, at least 60,500 kg/hr, at least 61,000 kg/hr, at least 61,500 kg/hr, at least 62,000 kg/hr, at least 62,500 kg/hr, at least 63,000 kg/hr, at least 63,500 kg/hr, at least 64,000 kg/hr, at least 64,500 kg/hr, at least 65,000 kg/hr, at least 65,500 kg/hr, at least 66,000 kg/hr, at least 66,500 kg/hr, at least 67,000 kg/hr, at least 67,500 kg/hr, at least 68,000 kg/hr, at least 68,500 kg/hr, at least 69,000 kg/hr, at least 69,500 kg/hr, at least 70,000 kg/hr, at least 70,500 kg/hr, at least 71,000 kg/hr, at least 71,500 kg/hr, at least 72,000 kg/hr, at least 72,500 kg/hr, at least 73,000 kg/hr, at least 73,500 kg/hr, at least 74,000 kg/hr, at least 74,500 kg/hr, at least 75,000 kg/hr, at least 75,500 kg/hr, at least 76,000 kg/hr, at least 76,500 kg/hr, at least 77,000 kg/hr, at least 77,500 kg/hr, or at least 78,000 kg/hr. In some embodiments, the third mass flow rate of the subset of the synthesis gas through the methanol synthesis reactor is at most 20,000 kg/hr, at most 20,500 kg/hr, at most 21,000 kg/hr, at most 21,500 kg/hr, at most 22,000 kg/hr, at most 22,500 kg/hr, at most 23,000 kg/hr, at most 23,500 kg/hr, at most 24,000 kg/hr, at most 24,500 kg/hr, at most 25,000 kg/hr, at most 25,500 kg/hr, at most 26,000 kg/hr, at most 26,500 kg/hr, at most 27,000 kg/hr, at most 27,500 kg/hr, at most 28,000 kg/hr, at most 28,500 kg/hr, at most 29,000 kg/hr, at most 29,500 kg/hr, at most 30,000 kg/hr, at most 30,500 kg/hr, at most 31,000 kg/hr, at most 31,500 kg/hr, at most 32,000 kg/hr, at most 32,500 kg/hr, at most 33,000 kg/hr, at most 33,500 kg/hr, at most 34,000 kg/hr, at most 34,500 kg/hr, at most 35,000 kg/hr, at most 35,500 kg/hr, at most 36,000 kg/hr, at most 36,500 kg/hr, at most 37,000 kg/hr, at most 37,500 kg/hr, at most 38,000 kg/hr, at most 38,500 kg/hr, at most 39,000 kg/hr, at most 39,500 kg/hr, at most 40,000 kg/hr, at most 40,500 kg/hr, at most 41,000 kg/hr, at most 41,500 kg/hr, at most 42,000 kg/hr, at most 42,500 kg/hr, at most 43,000 kg/hr, at most 43,500 kg/hr, at most 44,000 kg/hr, at most 44,500 kg/hr, at most 45,000 kg/hr, at most 45,500 kg/hr, at most 46,000 kg/hr, at most 46,500 kg/hr, at most 47,000 kg/hr, at most 47,500 kg/hr, at most 48,000 kg/hr, at most 48,500 kg/hr, at most 49,000 kg/hr, at most 49,500 kg/hr, at most 50,000 kg/hr, at most 50,500 kg/hr, at most 51,000 kg/hr, at most 51,500 kg/hr, at most 52,000 kg/hr, at most 52,500 kg/hr, at most 53,000 kg/hr, at most 53,500 kg/hr, at most 54,000 kg/hr, at most 54,500 kg/hr, at most 55,000 kg/hr, at most 55,500 kg/hr, at most 56,000 kg/hr, at most 56,500 kg/hr, at most 57,000 kg/hr, at most 57,500 kg/hr, at most 58,000 kg/hr, at most 58,500 kg/hr, at most 59,000 kg/hr, at most 59,500 kg/hr, at most 60,000 kg/hr, at most 60,500 kg/hr, at most 61,000 kg/hr, at most 61,500 kg/hr, at most 62,000 kg/hr, at most 62,500 kg/hr, at most 63,000 kg/hr, at most 63,500 kg/hr, at most 64,000 kg/hr, at most 64,500 kg/hr, at most 65,000 kg/hr, at most 65,500 kg/hr, at most 66,000 kg/hr, at most 66,500 kg/hr, at most 67,000 kg/hr, at most 67,500 kg/hr, at most 68,000 kg/hr, at most 68,500 kg/hr, at most 69,000 kg/hr, at most 69,500 kg/hr, at most 70,000 kg/hr, at most 70,500 kg/hr, at most 71,000 kg/hr, at most 71,500 kg/hr, at most 72,000 kg/hr, at most 72,500 kg/hr, at most 73,000 kg/hr, at most 73,500 kg/hr, at most 74,000 kg/hr, at most 74,500 kg/hr, at most 75,000 kg/hr, at most 75,500 kg/hr, at most 76,000 kg/hr, at most 76,500 kg/hr, at most 77,000 kg/hr, at most 77,500 kg/hr, or at most 78,000 kg/hr. In some embodiments, the third mass flow rate is between 44,000 lb/hr and 172,000 lb/hr, between 50,000 lb/hr and 170,00 lb/hr, between 70,000 lb/hr and 150,00 lb/hr, between 90,000 lb/hr and 130,00 lb/hr, or between 100,000 lb/hr and 110,00 lb/hr. In some embodiments, the third mass flow rate is at least 44,000 lb/hr, at least 54,000 lb/hr, at least 64,000 lb/hr, at least 74,000 lb/hr, at least 84,000 lb/hr, at least 94,000 lb/hr, at least 104,000 lb/hr, at least 114,000 lb/hr, at least 124,000 lb/hr, at least 134,000 lb/hr, at least 144,000 lb/hr, at least 154,000 lb/hr, at least 164,000 lb/hr, or at least 174,000 lb/hr. In some embodiments, the third mass flow rate is at most 44,000 lb/hr, at most 54,000 lb/hr, at most 64,000 lb/hr, at most 74,000 lb/hr, at most 84,000 lb/hr, at most 94,000 lb/hr, at most 104,000 lb/hr, at most 114,000 lb/hr, at most 124,000 lb/hr, at most 134,000 lb/hr, at most 144,000 lb/hr, at most 154,000 lb/hr, at most 164,000 lb/hr, or at most 174,000 lb/hr.

Block 350. Referring to block 350 of FIG. 3C, in some embodiments, the methanol product has a quality that includes less than 10 vol % impurities. For instance, in some embodiments, the methane product has a quality that includes less than 10 vol % inert impurities. In some embodiments, the methanol product includes less than 9.5 vol % inert impurities, less than 9 vol % inert impurities, less than 8.5 vol % inert impurities, less than 8 vol % inert impurities, less than 7.5 vol % inert impurities, less than 7 vol % inert impurities, less than 6.5 vol % inert impurities, less than 6 vol % inert impurities, less than 5.5 vol % inert impurities, less than 5 vol % inert impurities, less than 4.5 vol % inert impurities, less than 4 vol % inert impurities, less than 3.5 vol % inert impurities, less than 3 vol % inert impurities, less than 2.5 vol % inert impurities, less than 2 vol % inert impurities, less than 1.5 vol % inert impurities, less than 1 vol % inert impurities, less than 0.5 vol % inert impurities, or less than 0.1 vol % inert impurities.

In some embodiments, the methanol product has a water weight concentration (% W/W) of no more than 0.2% W/W water, of no more than 0.19% W/W water, of no more than 0.18% W/W water, of no more than 0.17% W/W water, of no more than 0.16% W/W water, of no more 0.15% W/W water, no more than 0.14% W/W water, of no more than 0.13% W/W water, of no more than 0.12% W/W water, of no more than 0.11% W/W water, of no more than 0.1% W/W water, of no more 0.09% W/W water, of no more 0.08% W/W water, of no more 0.07% W/W water, of no more 0.08% W/W water, of no more 0.05% W/W water, of no more 0.04% W/W water, of no more 0.03% W/W water, of no more 0.02% W/W water, or no more than 0.01% W/W water.

In some embodiments, the methanol product is a fuel grade methanol product. For instance, in some embodiments, the methanol product is stored (e.g., retained by a vessel or container instrument) and transported to a remote delivery point. However, the present disclosure is not limited thereto. Additional details and information regarding the quality of the methanol product is found at International Methanol Products and Consumers Association, 2021, “IMPCA Methanol Reference Specifications,” a.i.s.b.l. International Methanol Producers & Consumers Association i.v.z.w., Version 9, print, which is hereby incorporated by reference in its entirety for all purposes.

Block 352. Referring to block 352, in some embodiments, the method 300 further includes recycling the purge gas stream to a source of the reformer feed gas (e.g., stream 490 of FIG. 4, FIG. 492 of FIG. 4, block 214 of FIG. 2A, block 234 of FIG. 2B, block 304 of FIG. 3A, etc.). In some embodiments, the method further include recycling the purge gas stream to a source configured to utilize the purge gas stream for a beneficial use. Accordingly, the purge gas stream allows for the method to control an amount of impurities, such as inert compounds, that would otherwise accumulate in the methanol synthesis recycle loop.

In some embodiments, the purge gas stream includes hydrogen, carbon monoxide, carbon dioxide, one or more inert compounds, or a combination thereof. In some embodiments, the one or more inert compounds include nitrogen, argon, helium, neon, nitrous oxide, ozone, xenon, krypton, water vapor, chlorofluorocarbons, sulfur dioxide, nitrous oxide, or a combination thereof. For instance, in some embodiments, the one or more inert compounds have a substantially similar composition as atmospheric air excluding oxygen, since the atmospheric oxygen is consumed during the generation of the purge gas stream (e.g., block 340 of FIG. 3B). In some embodiments, the purge gas stream includes one or more non-methane volatile organic compounds (BVOCs). Additional details and information regarding the one or more inert compounds is found at Egger et al., 2003, “Composition of Earths Atmosphere,” Visionlearning, 5, print, which is hereby incorporated by reference in its entirety for all purposes.

In some embodiments, the providing the reformer feed gas further includes subjecting the reformer feed gas to an oxygen removal apparatus prior to methane reformer. In some embodiments, the oxygen removal apparatus includes a platinum or palladium mesh.

In some embodiments, the recycling the purge gas stream to the source has a fourth mass flow rate between 400 kg/h to 2,100 kg/h, between 450 kg/h to 2,050 kg/h, between 500 kg/h to 2,000 kg/h, between 550 kg/h to 1,950 kg/h, between 600 kg/h to 1,900 kg/h, between 650 kg/h to 1,850 kg/h, between 700 kg/h to 1,800 kg/h, between 750 kg/h to 1,750 kg/h, between 800 kg/h to 1,700 kg/h, between 850 kg/h to 1,650 kg/h, between 900 kg/h to 1,600 kg/h, between 950 kg/h to 1,550 kg/h, between 1,000 kg/h to 1,500 kg/h, between 1,050 kg/h to 1,450 kg/h, between 1,100 kg/h to 1,400 kg/h, between 1,150 kg/h to 1,350 kg/h, or between 1,200 kg/h to 1,300 kg/h. In some embodiments, the fourth mass flow rate of recycling the purge gas stream to the source is at least 450 kg/h, at least 500 kg/h, at least 550 kg/h, at least 600 kg/h, at least 650 kg/h, at least 700 kg/h, at least 850 kg/h, at least 900 kg/h, at least 950 kg/h, at least 1,000 kg/h, at least 1,050 kg/h, at least 1,100 kg/h, at least 1,150 kg/h, at least 1,200 kg/h, at least 1,250 kg/h, at least 1,300 kg/h, at least 1,350 kg/h, at least 1,400 kg/h, at least 1,450 kg/h, at least 1,500 kg/h, at least 1,550 kg/h, at least 1,600 kg/h, at least 1,650 kg/h, at least 1,700 kg/h, at least 1,750 kg/h, at least 1,800 kg/h, at least 1,850 kg/h, at least 1,900 kg/h, at least 1,950 kg/h, at least 2,000 kg/h, or at least 2,050 kg/h. In some embodiments, the fourth mass flow rate of recycling the purge gas stream to the source is at most 450 kg/h, at most 500 kg/h, at most 550 kg/h, at most 600 kg/h, at most 650 kg/h, at most 700 kg/h, at most 850 kg/h, at most 900 kg/h, at most 950 kg/h, at most 1,000 kg/h, at most 1,050 kg/h, at most 1,100 kg/h, at most 1,150 kg/h, at most 1,200 kg/h, at most 1,250 kg/h, at most 1,300 kg/h, at most 1,350 kg/h, at most 1,400 kg/h, at most 1,450 kg/h, at most 1,500 kg/h, at most 1,550 kg/h, at most 1,600 kg/h, at most 1,650 kg/h, at most 1,700 kg/h, at most 1,750 kg/h, at most 1,800 kg/h, at most 1,850 kg/h, at most 1,900 kg/h, at most 1,950 kg/h, at most 2,000 kg/h, or at most 2,050 kg/h. In some embodiments, the fourth mass flow rate is between 880 lb/hr and 4,600 lb/hr, between 1,000 lb/hr and 4,400 lb/hr, between 1,200 lb/hr and 4,200 lb/hr, between 1,400 lb/hr and 4,000 lb/hr, between 1,600 lb/hr and 3,800 lb/hr, between 1,800 lb/hr and 3,600 lb/hr, between 2,000 lb/hr and 3,600 lb/hr, between 2,200 lb/hr and 3,400 lb/hr, between 2,400 lb/hr and 3,200 lb/hr, or between 2,600 lb/hr and 3,000 lb/hr. In some embodiments, the fourth mass flow rate is at least 850 lb/hr, at least 900 lb/hr, at least 1,000 lb/hr, at least 1,100 lb/hr, at least 1,200 lb/hr, at least 1,300 lb/hr, at least 1,400 lb/hr, at least 1,500 lb/hr, at least 1,600 lb/hr, at least 1,700 lb/hr, at least 1,800 lb/hr, at least 1,900 lb/hr, at least 2,000 lb/hr, at least 2,100 lb/hr, at least 2,200 lb/hr, at least 2,300 lb/hr, at least 2,400 lb/hr, at least 2,500 lb/hr, at least 2,600 lb/hr, at least 2,700 lb/hr, at least 2,800 lb/hr, at least 2,900 lb/hr, at least 3,000 lb/hr, at least 3,100 lb/hr, at least 3,200 lb/hr, at least 3,300 lb/hr, at least 3,400 lb/hr, at least 3,500 lb/hr, at least 3,600 lb/hr, at least 3,700 lb/hr, at least 3,800 lb/hr, at least 3,900 lb/hr, at least 4,000 lb/hr, at least 4,100 lb/hr, at least 4,200 lb/hr, at least 4,300 lb/hr, or at least 4,400 lb/hr. In some embodiments, the fourth mass flow rate is at most 850 lb/hr, at most 900 lb/hr, at most 1,000 lb/hr, at most 1,100 lb/hr, at most 1,200 lb/hr, at most 1,300 lb/hr, at most 1,400 lb/hr, at most 1,500 lb/hr, at most 1,600 lb/hr, at most 1,700 lb/hr, at most 1,800 lb/hr, at most 1,900 lb/hr, at most 2,000 lb/hr, at most 2,100 lb/hr, at most 2,200 lb/hr, at most 2,300 lb/hr, at most 2,400 lb/hr, at most 2,500 lb/hr, at most 2,600 lb/hr, at most 2,700 lb/hr, at most 2,800 lb/hr, at most 2,900 lb/hr, at most 3,000 lb/hr, at most 3,100 lb/hr, at most 3,200 lb/hr, at most 3,300 lb/hr, at most 3,400 lb/hr, at most 3,500 lb/hr, at most 3,600 lb/hr, at most 3,700 lb/hr, at most 3,800 lb/hr, at most 3,900 lb/hr, at most 4,000 lb/hr, at most 4,100 lb/hr, at most 4,200 lb/hr, at most 4,300 lb/hr, or at most 4,400 lb/hr.

### Example 2: A Steam of Unsorted MSW

In some embodiments, a stream of unsorted MSW was obtained from one or more sources that included a first source associated with a first municipality with a first population of about 1.32 million subjects and a second source associated with a second municipality with a second population of about 611,000 subjects. Table 1 below provides a listing of the stream of unsorted MSW from the first source included:

TABLE 1
Unsorted MSW from First Source

| Material | tons per year |
|---|---|
| Organic matter | 153,224 |
| Private wood waste and | 18,402 |
| gardening waste | |
| Paper and cardboard | 43,939 |
| Tetra brick | 2,628 |
| Metal | 7,135 |
| Plastic PEBD | 30,420 |
| Plastic bottles | 7,887 |
| Plastic (other) | 28,917 |
| Glass | 13,144 |
| Textile | 10,140 |
| Debris | 30,795 |
| Hazardous waste | 1,127 |
| Composite materials | 27,791 |
| Public wood waste and | 110,739 |
| gardening waste | |
| Total: | 486,289 |

Accordingly, the first source provided about 1.01 kg of MSW per day.

Table 2 below provides a listing of the stream of unsorted MSW from the second source included:

TABLE 2
Unsorted MSW from Second Source

| Material | tons per year |
|---|---|
| Organic matter | 47,054 |
| Private wood waste and | 26,128 |
| gardening waste | |
| Paper and cardboard | 19,413 |
| Tetra brick | 1,270 |
| Metal | 11,811 |
| Plastic PEBD | 18,839 |
| Plastic bottles | 5,309 |
| Plastic (other) | 9,289 |
| Glass | 5,358 |
| Textile | 0 |
| Debris | 2,261 |
| Hazardous waste | 2,453 |
| Composite materials | 24,771 |
| Public wood waste and | 23,302 |
| gardening waste | |
| Total: | 196,254 |

Accordingly, the second source provided about 0.88 kg of MSW per day.

### Example 2: A System for Producing Municipal Solid Waste

In an embodiment of the present disclosure a system for producing methanol was provided.

The system included a sorting mechanism (e.g., a sorting mechanism instrument 110 of FIG. 1). The sorting mechanism was configured to process a stream of unsorted municipal solid waste (MSW), which produced a stream of sorted MSW. The stream of sorted MSW included a first portion substantially including digestible biological solids.

The system further included an anaerobic digestion apparatus. The anaerobic digestion apparatus was configured to retain a solution for an epoch (e.g., block 214 of FIG. 2A). The solution included the first portion of the stream of sorted MSW and a second portion including a first product of a methanol synthesis reactor, which produced a reformer feed gas. The reformer feed gas included methane and carbon dioxide, in which the methane as present in the reformer feed gas in an amount of between 20 vol % and 90 vol % and the carbon dioxide was present in the reformer feed gas in an amount of between 10 vol % and 80 vol %.

Moreover, the system included a steam methane reformer configured to treat the reformer feed gas, which produced a synthesis gas. The synthesis gas included a first portion of hydrogen, a second portion of carbon monoxide, and a third portion of carbon dioxide. Furthermore, the system included the methanol synthesis reactor configured to produce a methanol product and the first product from the synthesis gas.

In some embodiments, the steam methane reformer utilized as an energy source a first portion of the stream of unsorted MSW including a wood waste. In some embodiments, the steam methane reformer utilized as the energy source includes a landfill gas stream. In some embodiments, the steam methane reformer utilized as the energy source includes a portion of the biogas stream generated by anaerobic digestion.

In some embodiments, anaerobic digestion apparatus was configured to produce a second product that includes a solid. Moreover, the steam methane reformer utilized as the energy source the second product. In some embodiments, the second product included a slurry including one or more solid waste products. In some embodiments, the second product was subjected to a pyrolysis process task 114, which produced a biochar (e.g., product 476 of FIG. 4) and/or a reformer feed gas (e.g., product 478 and/or product 484 of FIG. 4). In some embodiments, the biochar is a high-carbon (e.g., C>2, C>3, C>4, C>6, etc.) solid that is formed as a product of the pyrolysis task 114 or incineration task 114. In some embodiments, the solid of the second product was a thermal decomposition of the solution (e.g., an anaerobic digestate). Additional details and information regarding producing biochar is found at Downie et al., 2011, “Biochar Production and Use: Environmental Risks and Rewards,” University of New South Wales, School of Materials Science and Engineering, print, which is hereby incorporated by reference in its entirety for all purposes.

In some embodiments, for the synthesis gas, the first portion of hydrogen was between 60 vol % and 78 vol %, the second portion of carbon monoxide was between 14 vol % and 33 vol %, and the third portion of carbon dioxide was between 5 vol % and 20 vol %. Furthermore, in some embodiments, for the synthesis gas, the first portion of hydrogen was between 66 vol % and 78 vol %, the second portion of carbon monoxide was between 14 vol % and 33 vol %, and the third portion of carbon dioxide was between 5 vol % and 20 vol %. In some embodiments, for the synthesis gas, the first portion of hydrogen was between 60 vol % and 78 vol %, the second portion of carbon monoxide was between 14 vol % and 33 vol %, and the third portion of carbon dioxide was between 5 vol % and 20 vol %. Furthermore, in some embodiments, for the synthesis gas, the first portion of hydrogen was between 66 vol % and 78 vol % and the second portion of carbon monoxide was between 20 vol % and 25 vol %.

### Example 3: Systems and Methods for Producing Methanol from Municipal Solid Waste

In some embodiments, a stream of unsorted MSW was obtained from one or more sources, such as one or more agriculture sources and/or one or more landfill sources. In some embodiments, the stream of unsorted MSW was obtained at a rate of about 12,000 tons per day. In some embodiments, the stream of unsorted MSW was processed by one or more sorting mechanisms in order to separate metals from other material. In some embodiments, the separated metals were provided back to the one or more sources, such as the one or more landfill sources, in order to extend the usable life of the metal and reduce a capacity of material at the one or more landfill sources. For instance, referring to FIG. 4, in some embodiments, a first portion of the steam of sorted MSW 454 that included metal is provided to a first source (e.g., recycling system source) and/or a second portion of the stream of sorted MSW 456 that included polymers is provided to a second source (e.g., landfill source). In some embodiments, the processing by the one or more sorting mechanisms provided a stream of sorted MSW, which included an organic fraction prepared as feedstock solution for utilization with an anaerobic digestion process task. In some embodiments, the solution the MSW accounted for about 10 vol % of the solution with a total volume of about 50,000 m³. After about 35 to 55 days, the anaerobic digestion process task produced a reformer feed gas that included about 62 vol % methane and about 38 vol % carbon dioxide. The reformer feed gas was treated (e.g., purified of contaminant impurities) and provided to a methane steam reformer to produce a synthesis gas. In some embodiments, the synthesis gas consisted of between 20 vol % and 5 vol % carbon dioxide (e.g., about 6 vol % carbon dioxide). In some embodiments, the reformer feed gas was provided at a first mass flow rate of about 9,750 kg/h. The synthesis gas was provided to a methanol synthesis reactor that included a recycle loop. In some embodiments, the synthesis gas was provided at a second mass flow rate of about 31,500 kg/h. The recycle loop was used to pass a subset of the synthesis gas through the methanol synthesis reactor for two or more passes. Accordingly, the systems and methods of the present disclosure produced from about 60 tons to about 250 tons of fuel grade methanol (e.g., less than 4 vol % inert impurities) in one day. A carbon efficiency of the systems and methods of the present disclosure was between 96% and 99%.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain the program modules shown in any combination of the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of producing a reformer feed gas, the method comprising:
A) obtaining, from one or more sources, a stream of unsorted municipal solid waste (MSW);
B) processing, by one or more sorting mechanisms, the stream of unsorted MSW, thereby producing a stream of sorted MSW; and
C) retaining, for an epoch, a solution comprising a first portion of the stream of sorted MSW in an anaerobic digestion apparatus, wherein the first portion substantially comprises digestible biological solids, thereby producing the reformer feed gas through anaerobic digestion of the solution in the anaerobic digestion apparatus.

2. The method of claim 1, wherein the stream of unsorted MSW comprises a food waste, a garden waste, a wood waste, a crop waste, a food manufacture byproduct, a slaughterhouse waste, a used food fat, a used edible oil, a used grease from food, a manure, a biosolid, a sewage sludge, or a combination thereof.

3. The method of claim 2, wherein the stream of unsorted MSW comprises from about 10 weight percent (wt %) to about 30 wt % wood waste.

4. The method of claim 1, wherein retaining C) produces the reformer feed gas has at a mass flow rate between 6,000 kg/h and 15,000 kg/h.

5. The method of claim 1, wherein the first portion of the stream of sorted MSW comprises greater than or equal to 90 wt % biological waste.

6. The method of claim 1, wherein the stream of unsorted MSW consists of between 1,000 tons and 15,000 tons of material per day.

7. The method of claim 1, wherein the one or more sorting mechanisms comprises one or more mechanical sorting mechanisms, one or more electromechanical sorting mechanisms, one or more manual sorting mechanisms, or a combination thereof.

8. The method of claim 7, wherein the one or more sorting mechanisms comprises a hopper sorting mechanism, a bag opening sorting mechanism, a sizing filter mechanism, a shredding sorting mechanism, a trommel sorting mechanism, a three-dimensional sorting mechanism, an optical sorting mechanism, a ballistic sorting mechanism, a magnetic sorting mechanism, an eddy current sorting mechanism, a hydrothermal digestible material extraction mechanism, or a combination thereof.

9. The method of claim 1, further comprising
D) treating, by a conversion process, the reformer feed gas, thereby producing a treated reformer feed gas.

10. The method of claim 9, wherein
a carbon dioxide content of the reformer feed gas is between 35 vol % and 45 vol %, and
a carbon dioxide content of the treated reformer feed gas is between 20 vol % and 23 vol %.

11. The method of claim 9, wherein the conversion process comprises removing a first portion of the reformer feed gas that is nitrogen, a second portion of the reformer feed gas that is oxygen, a third portion of the reformer feed gas that is one or more silicon compounds, a fourth portion of the reformer feed gas that is sulfur or one or more sulfur compounds, or a combination thereof.

12. The method of claim 9, wherein the conversion process comprises one or more dehydration processes, one or more fixed media bed processes, one or more chilling processes, one or more gas compression processes, or a combination thereof.

13. The method of claim 1, wherein the anaerobic digestion apparatus comprises a slurry stir mechanism configured to continuously or intermittently agitate the solution for the epoch.

14. The method of claim 1, wherein the epoch is from about 5 days to about 55 days.

15. The method of claim 1, wherein, during the retaining C), the solution is at a temperature of from about 85 degrees Fahrenheit (° F.) to about 105° F.

16. The method of claim 1, wherein, during the retaining C), the solution is at a temperature of from about 115° F. to about 130° F.

17. The method of claim 1, wherein the solution comprises from about 5 wt % solid concentration to about 25 wt % solid concentration of the first portion of the stream of sorted MSW.

18. The method of claim 1, wherein the solution comprises a purge gas stream of a methanol synthesis reactor recycled to the solution.

19. The method of claim 18, wherein the purge gas stream comprises a hydrogen gas mixture.

20. The method of claim 1, wherein the solution comprises wastewater from a methanol purification apparatus.

21. The method of claim 1, wherein the solution has a total volume of between 40,000 cubic meters (m³) and 100,000 m³.
